(19) United States
(12) Patent Application Publication
(10) Pub. No.: US 2026/0061092 A1
(43) Pub. Date: Mar. 5, 2026

(54) MINERALIZATION OF CELL-LADEN MATRICES

(71) Applicant: Oregon Health & Science University, Portland, OR (US)

(72) Inventor: Luiz E. Bertassoni, Portland, OR (US)

(21) Appl. No.: 18/933,905

(22) Filed: Oct. 31, 2024

Related U.S. Application Data
(63) Continuation of application No. 17/279,437, now abandoned.
(60) Provisional application No. 62/736,404, filed on Sep. 25, 2018.
(60) Provisional application No. 62/864,935, filed on Jun. 21, 2019.

(51) Int. Cl. A61L 27/04; A61L 27/12; A61L 27/24; A61L 27/38

(52) U.S. Cl. CPC A61L 27/04; A61L 27/12; A61L 27/24; A61L 27/3834; A61L 2430/02

(57) ABSTRACT

This disclosure relates to methods of mineralizing cell-laden matrices. Disclosed herein are cell-laden matrix compositions. Also disclosed herein are methods of selectively mineralizing a cell-laden matrix. Methods of culturing biomimetic bone tissue are disclosed herein. Also disclosed herein are kits containing compositions disclosed herein or portions thereof.

### RELATED APPLICATIONS

This application is continuation of U.S. patent application Ser. No. 17/279,437, filed Mar. 24, 2021, which is a National Stage of International Application No. PCT/US2019/053022, filed Sep. 25, 2019, which claims priority to U.S. Provisional Patent Application No. 62/736,404, filed on Sep. 25, 2018, and titled “DIRECTED INTRAFIBRILLAR MINERALIZATION OF CELL-LADEN COLLAGEN” and U.S. Provisional Patent Application No. 62/864,935, filed on Jun. 21, 2019, and titled “DIRECTED INTRAFIBRILLAR MINERALIZATION OF CELL-LADEN COLLAGEN,” the entire disclosures of which are hereby incorporated herein by reference.

### FEDERALLY SPONSORED RESEARCH OR DEVELOPMENT

This invention was made with U.S. government support under NIH R01 Grant No. DE026170-01 awarded by the National Institutes of Health. The U.S. government has certain rights in the invention.

### COPYRIGHT NOTICE

2019 Oregon Health and Science University. A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR § 1.71 (d).

### TECHNICAL FIELD

The present disclosure relates to methods of mineralization. More particularly, the disclosure relates to methods of mineralizing cell-laden matrices.

### SEQUENCE LISTING

The Sequence Listing is submitted as an XML file in the form of the file named “Sequence.xml” (12,000 bytes), which was created on Nov. 14, 2025, which is incorporated by reference herein.

### BACKGROUND

Regenerating human bone is one of the greatest challenges in the medical field in the 21^{st} century. Bone-related diseases and injuries yield an annual economic burden of approximately $800 billion/year in the US alone. Although outstanding progress has been made in understanding the intricate biology of stem cell differentiation in bone regeneration, replicating the overall complexity of human bone in-vitro has been one of the greatest limitations in regenerative medicine. Current strategies to engineer bone rely on the use of simple synthetic ceramic materials or soft hydrogel scaffolds; but both grossly fail to replicate the highly intricate structure, composition and mechanics of native bone.

In native bone, a natural organic scaffold (mostly collagen and other proteins) functions synergistically with cell-mediated biomineralization during bone formation. As a result of such interactions, the organic matrix becomes strengthened by hydroxyapatite crystallites that are positioned both inside (intrafibrillar mineral) and outside (extrafibrillar mineral) of the fibrillar protein with the aid of non-collagenous proteins (NCPs). This partitioning, especially the so-called intrafibrillar mineral, is responsible for the outstanding mechanical properties and longevity of mineralized tissues in the body. Most importantly, it is in this highly complex mineralized matrix microenvironment where over 90% of bone cells reside. To date no strategy has been able to replicate such a complex 3D cell-laden and mineralized microenvironment. This is primarily due to the fact there has been no chemistry that allows for cells to be cultured in a Ca and P rich mineralizing condition leading to biomimetic mineralization like human bone.

The critical relevance of this knowledge gap is that cells respond to their extracellular environment depending on its composition, structure, and mechanics. Consequently, cells do not behave in near in-vivo conditions if they are simply cultured two-dimensionally on ceramic scaffolds or on a Petri-dish. Neither do they behave physiologically when embedded in hydrogels that lack the key hallmarks of the bone extracellular matrix. Additionally, it had been thought in the art that the increased osmotic pressure of Ca and P rich mineralizing conditions could lead to cell lysis, precluding culture of living cells in Ca and P rich mineralizing conditions. Thus, engineering such complex microenvironments is a major challenge that has arguably hampered the effective translation of bone regeneration into clinical practice and a systematic understanding of the mechanisms behind bone regeneration.

### BRIEF DESCRIPTION OF THE DRAWINGS

The embodiments disclosed herein will become more fully apparent from the following description and appended claims, taken in conjunction with the accompanying drawings.

FIG. 1a shows SEM images of non-mineralized (left) and mineralized (right) hMSC-laden collagen hydrogels showing the formation of mineralized collagen fibril bundles. Extrafibrillar mineral is apparent. Scale bar: 400 nm.

FIG. 1b are images of collagen hydrogels prior to (left) and immediately after a 3-day mineralization period (right). The mineral formation resulted in a white opaque appearance.

FIG. 1c is a graphical representation of the EDX spectra of mineralized samples confirming the presence of Ca and P in mineralized specimens (right), and lack thereof in non-mineralized controls (left).

FIG. 1d depicts TEM images of non-mineralized collagen (left), mineralized collagen (middle), and a zoomed view of a single mineralized fibril (right). Far right panel depicts selective area electron diffraction (SAED) of a mineralized collagen showing the typical broad arcs for the (002) plane and overlapping arcs for the (112), (211), and (300) planes that are consistent with the known hexagonal crystalline structure of hydroxyapatite. Non-mineralized collagen shows the typical D-banding pattern of collagen. The banding becomes obscured in the mineralized collagen, which appears to have both intra and extrafibrillar mineral crystallites (right and far right). Scale bar: 500 nm. The zoomed view of a single mineralized fibril (far right) suggests the preferential orientation of apatite crystallites (dark streaks) parallel to the c-axis of the fibril. Scale bar: 50 nm

FIG. 1e is a FTIR spectrum representative of the analyses of mineralized matrix, non-mineralized matrix, and native bone.

FIG. 1f is a graph showing the respective mineral: matrix ratio of mineralized versus non-mineralized constructs, compared to that of native bone (**** p<0.0001, ANOVA/Tukey). Mineralized samples had comparable values to that of native bone, and both were significantly higher than non-mineralized controls (**** p<0.0001 ANOVA/Tukey).

FIG. 1g is a graphical representation of reactive oxygen species levels. Mineralization of cell-laden hydrogels resulted in non-significant generation of reactive oxygen species (ROS) compared to H₂O₂ (**** p<0.0001, ANOVA/Tukey).

FIG. 1h is a graphical representation of cell viability. Cell viability levels were consistently above 90% after at least 7 days of culture in all samples (*p<0.05, Student's t-test).

FIG. 1i is a graphical representation of the mineral: matrix ratio (**** p<0.0001, Student's t-test) of mineralized and non-mineralized constructs, dotted lines are reference values of native bone.

FIG. 1j is a graphical representation of the crystallinity index (*** p<0.001, Student's t-test) of mineralized and non-mineralized collagen constructs, dotted lines are reference values of native bone. Mineral crystallinity calculated from the FT-IR spectra suggestive of native bone-like apatite crystallinity in mineralized microenvironment. Crystallinity index was calculated from the extent of splitting of the two absorption bands at 605 and 565 cm-1. (N=6).

FIG. 1k is a graphical representation of AFM nanoindentation modulus of non-mineralized and mineralized hydrated collagen fibrils.

FIG. 2 depicts 3D volumetric reconstruction of backscattered high-resolution electron micrographs obtained via serial block-face SEM of cells embedded in a mineralized collagen hydrogel after 7 days of culture. Panel a (left) illustrate the serial stacking of 190 60 nm-thin sections. The middle panel illustrates the segmentation of cells (blue) from the surrounding mineralized matrix, and the right panel illustrates the visualization of block 3D image. The darker features surrounding cells indicate a higher-density of backscatter contrast, suggestive of more heavily mineralized collagen, and light features are indicative of non-mineralized regions Panel b depicts a 3D rendered image showing cells (blue) embedded in mineral (red), with the underlying collagen (grey). Panel c depicts a 3D rendered image with the exclusion of collagen via digital processing illustrating the density of mineralized collagen, and cells spreading within a bed of mineralized matrix. Panel d depicts two higher magnification images that show narrow cell processes (blue) appearing to extend between mineralized fibrils. Panel e depicts a 3D rendered image with digital removal of cell bodies from within the mineralized matrix illustrating the increased density of mineral surrounding the cell structure.

FIG. 3 depicts in panels a-i images of cells imbedded within the non-mineralized hydrogel, mineralized collagen, and OIM treated samples. Panels a and c depict single block face backscatter SEM image of a cell embedded within the (panel a) non-mineralized hydrogel, (panel b) a cell embedded in mineralized collagen and (panel c) OIM-treated sample. The pericellular empty space surrounding individual cell bodies in (panel b), which is reminiscent of lacunae in osteonal bone, is visibly bordered by regions of apparently higher density mineralized collagen, resembling the native bone's lamina-limitans. Panels d-i depict reflectance confocal microscopy image of collagen (panels g-i) and F-Actin/DAPI (panels d-f) stained hMSCs in both non-mineralized (panels d and g) and mineralized (panel e and h) collagen. Panels d-f are showing dendritic-like extensions of cells after matrix mineralization, reminiscent of an osteocyte-like morphology with a network constructed for cell-cell communication. Cells in non-mineralized hydrogels had little effect on matrix (red) remodeling, whereas mineralized samples showed well-defined lacunae-like spaces (dark), consistent with the morphology of the cells residing in them. Scale bar: 100 μm. Panels j-k graphically represent morphological characteristics of cells in mineralized versus non-mineralized constructs. Panel j is a graphical representation of the number dendritic-like projections. Panel k is a graph representing the length of dendritic-like projections. Cells within the mineralized hydrogels exhibited higher number of dendritic-like projections compared to cells in non-mineralized constructs, which had relatively fewer and shorter extensions. Average length of dendritic-like projections per cell was also significantly higher in mineralized constructs than in non-mineralized hydrogels (**** p<0.0001, Student's t-test).

FIG. 4a is a graphical representation of the results from the gene expression analyses of hMSCs cultured in non-mineralized versus mineralized constructs (without osteoinductive supplements), compared to cells cultured in osteoinductive medium (OIM, positive control). The fold change of runt-related transcription factor 2 (RUNX2; top left), alkaline phosphatase (ALP; top middle), osteocalcin (OCN; top right), podoplanin (PDPN; bottom left), and dentin matrix protein 1 (DMP1; bottom right) are shown. Expression of osteogenic differentiation marker, osteocalcin (OCN) was significantly higher (p<0.01) in mineralized collagen than in collagen supplemented with osteoinductive medium after 21 days. Expression of osteocyte-related markers (dentin matrix protein 1-DMP1 and podoplanin-PDPN) was comparable after 21 days of cell culture in mineralized scaffolds to the positive control (osteoinductive medium), but significantly higher in mineralized collagen at earlier time points (DMP1, p<0.01 after 7 days, and p<0.05 after 14 days) (*p<0.05, ** p<0.01, *** p<0.001, **** p<0.0001 ANOVA/Tukey).

FIG. 4b is a graph of the relative expression of bone metabolism-related proteins by hMSCs in mineralized collagen versus osteoinductive medium and non-mineralized controls. A significant increase in the expression of BMP 2 and BMP 6 for cells in both mineralized collagen and in osteoinductive medium relative to non-mineralized controls is consistent with enhanced bone-specific metabolic activity. A marked increase in the ratio of RANKL/OPG in mineralized samples, however, suggests the stronger potency for cell-mediated bone-remodeling via a paracrine signaling in cell-laden mineralized constructs than in the other groups (*p<0.05, ** p<0.01, *** p<0.001, **** p<0.0001 ANOVA/Tukey).

FIG. 4c depicts representative Alizarin red staining images of non-mineralized, mineralized, and OIM constructs in panels from the left to right. The extensive and homogeneous mineral deposition within 7 days of culture in the mineralized constructs, as compared to the sparse mineral nodules in the positive control, and absence of mineralization in non-mineralized controls are depicted. Scale bar: 100 μm.

FIG. 4d depicts images of the expression of the osteogenic differentiation marker OCN (14 days) by cells cultured in mineralized samples (middle panel) versus non-mineralized controls (left panel) and osteoinductive medium (right panel). Scale bar: 200 μm.

FIG. 4e depicts images of the expression of the osteocyte-specific marker PDPN (14 days) by cells cultured in mineralized samples (middle panel) versus non-mineralized controls (left panel) and osteoinductive medium (OIM; right panel). Scale bar: 200 μm.

FIG. 4f depicts images of the surface expression of DMP1. The expression is comparable for (right panel) OIM-treated cells (**** p<0.0001) and (middle panel) mineralized collagen (*p<0.05), both of which were significantly higher than (left panel) non-mineralized controls (N=4). Scale bar: 200 μm.

FIG. 5a depicts the timeline for culture of pre-vascularized and mineralized bone-like tissue constructs, followed by subcutaneous implantation in SCID mice.

FIG. 5b depicts immunofluorescence images of vascularized constructs subjected to non-mineralization control (top panels) or mineralization (bottom panels). Images show representative fluorescence for αSMA (left panels), αSMA, F-actin, and DAPI (panels second from the left), CD31 and DAPI (panels third from the left), and RUNX2 and GFP (right panels). HUVECs formed interconnected endothelial networks which were supported by αSMA-expressing hMSCs tightly adhered to forming vessels. Staining with F-Actin and DAPI are also shown. Scale bar: 50 μm. The network structures were also positive for the endothelial cell surface marker CD31 (Scale bar: 400 μm), while the remainder of hMSCs expressed RUNX2 as a marker for osteogenic differentiation. Scale bar: 50 μm.

FIG. 5c depicts Von kossa staining images of the non-mineralized (top panels) versus mineralized tissue sections (bottom panels) after 7 days of in vitro culture and 7 days of implantation. The dark/brown staining indicates areas of dense calcification in the mineralized tissue sections, whereas the non-mineralized tissue sections illustrate negative staining for Von Kossa. Images on the right panels show higher magnification Von Kossa stained image of the engrafted construct shown in the left panels. The presence of numerous lumens in the mineralized sections reveal microvessel formation within the calcified construct.

FIG. 5d are images of non-mineralized (upper panels) and mineralized constructs (lower panels) stained with H&E (left panels), anti-human CD31 antibody (middle panels), and anti-αSMA antibody (right panels). H&E image depicts the collagenous matrix populated with cells. Anti-human CD31 antibody staining suggested the formation of endothelial networks by the transplanted HUVECs, and which are not due to host blood vessel infiltration. The vessels in the non-mineralized sections show signs of regression with constricted lumens, as opposed to the well-defined HUVEC-lined vessel structures in the mineralized construct. Anti-αSMA staining shows fewer αSMA+ cells in the non-mineralized sections, whereas most of the vessels in the mineralized construct appear to be wrapped by pericyte-like cells.

FIG. 5e graphically represent the results from the analysis of vessel numbers (left panel), vessel diameter (panel second from the left), CD31 (panel third from the left), and αSMA (far right) in non-mineralized and mineralized constructs. Quantification of the vessel parameters (vessel number and diameter) indicate robust vascularization and cell survival in mineralized groups, compared to their non-mineralized controls. Quantitative analysis of % area of CD31 and αSMA immunostaining suggests an increased vascularization and vessel stabilization in mineralized constructs. (**** p<0.0001 Student's t-test).

FIG. 5f depicts representative bioluminescence images captured before and once a week until 3 weeks (panels from left to right), after injection of PC3/Luc cells. Local invasion of PC3 cells at the ectopic site implanted with 3D mineralized construct. The non-mineralized and mineralized constructs were subcutaneously implanted on the left and right flank of immunocompromised mice, followed by direct injection of luciferase expressing PC3 cells (1×10⁵ cells), 24 h post implantation. A much higher bioluminescence signal intensity was detected in the region implanted with mineralized construct compared to the non-mineralized control.

FIG. 5g is a graph representing the quantification of the bioluminescence signal intensity (depicted as total photon flux) in non-mineralized versus mineralized groups of mice. Error bars indicate SEM; * P<0.05.

FIG. 6a depicts the timeline for the generation of innervated and mineralized collagen constructs. Human SH-SY5Y neuroblastoma cells were co-encapsulated with hMSCs (4:1) in non-mineralized and mineralized hydrogels, after 14 days of differentiation. For the first 7 days, the cells were treated in DMEM/F12 basal medium supplemented with 1% FBS and 10 UM all-trans retinoic acid (RA), followed by an additional 7 days of culture in Neurobasal-A medium containing 1% (v/v) L-glutamine, 1X B-27 supplement, 50 ng/ml human BDNF and 10 UM RA. Subsequently, matrix mineralization was triggered by switching the neurobasal medium for the mineralizing medium for another 3 days.

FIG. 6b depicts representative immunofluorescence images showing the expression of NSE in non-mineralized (upper panels) and mineralized (lower panels) constructs. The fully differentiated neuronal cells within the mineralized constructs were confirmed by immunostaining with antibodies against Neuron specific enolase (NSE). Cell nuclei were counter-stained with DAPI (middle panels) and cytoskeletal actin was stained with AlexaFluor 488 phalloidin (right panels). The neuronal differentiation markers had similar expression levels in mineralized and non-mineralized groups. Scale bar: 50 μm.

FIG. 6c depicts representative immunofluorescence images showing the expression of NEFL in non-mineralized (top panels) and mineralized (bottom panels) constructs. The fully differentiated neuronal cells within the mineralized constructs were confirmed by immunostaining with antibodies against Neurofilament light (NEFL). Cell nuclei were counter-stained with DAPI (middle panels) and cytoskeletal actin was stained with AlexaFluor 488 phalloidin (right panels). The neuronal differentiation markers had similar expression levels in mineralized and non-mineralized groups. Scale bar: 50 μm.

FIG. 6d are graphical representations of the results from the analysis of the morphological parameters of the differentiated SHSY-5Y cells in non-mineralized versus mineralized constructs. Imaris Filament Tracer module was used to quantify the parameters. Quantification of the number of neurites (far left), total neurite length (second from left), and maximum length of neurites (second from right) indicate no significant difference between non-mineralized and mineralized groups, whereas the number of branches and branch points (far right) was higher in the mineralized groups. Data are represented as Mean±SD. * p<0.05. (N=4).

FIG. 7 is a graphical representation of the effect of mineralizing medium on cell cytotoxicity. Cytotoxicity was determined using an MTT (3-(4,5-Dimethylthiazol-2-yl)-2,5-Diphenyltetrazolium Bromide) assay kit on an adherent monolayer of hMSCs exposed to mOPN alone, and varying concentrations of calcium and phosphate-supplemented medium, with or without mOPN as a mineralization process-directing agent. Untreated cells cultured on tissue-culture plastic were used as a control. Briefly, hMSCs were seeded in a 96 well plate at a density of 5×10³ cells/well and the following day, cells were treated. After 48 hrs of incubation, the MTT reagent was added and the samples were incubated at 37° C. for 4 hours. The resulting purple colored formazan crystals were dissolved in DMSO and the absorbance was measured spectrophotometrically at 570 nm using a microplate reader. Exposure to increasing concentrations of calcium and phosphate containing medium resulted in a significant reduction in cell viability. In contrast, treatment with mOPN alone did not affect the cell viability, except for a slight reduction at a concentration of 500 μg/mL. Interestingly, the concentration dependent cytotoxicity response of calcium and phosphate mineralizing medium was significantly mitigated in the presence of mOPN, even at the highest concentration tested. Such a negated cytotoxicity effect of calcium and phosphate when used in combination with mOPN may possibly be due to the formation of amorphous calcium phosphate precursors in the culture medium which reduce free CaP ion-induced cellular damage by preventing its intracellular uptake. Data presented as Mean±SD. Data points connected by similar color bars represent a significant difference within the groups (p<0.05), whereas data points underlined with #indicate a significant difference between groups.

FIG. 8 depicts SEM micrographs showing the cells encapsulated in (left panel) non-mineralized and (right panel) mineralized collagenous matrix after 7 days. Cells appeared well-spread, exhibiting multiple contacts with the underlying matrix as well as with the neighboring cell bodies. Cells in mineralized samples appeared slightly rougher than cells in non-mineralized controls, suggesting the possibility of mineral deposition onto the cell surface as well as the extracellular matrix. Scale bars: 10 μm.

FIG. 9 depicts a SEM image showing the mineralized collagenous matrix of human bone. The absence of characteristic D-periodicity in collagen fibrils suggest mineral deposition within the intrafibrillar space. The presence of extrafibrillar mineral surrounding and encasing the densely packed fibril bundles is also noticeable. Scale bar: 1 μm.

FIG. 10 panel a depicts a TEM image of mineralized collagen (unstained) lyophilized and pulverized in liquid nitrogen. Scale bar: 500 nm. Panel b depicts a high magnification image from white dotted square in panel a. Needle shaped apatite crystals occupying both the intrafibrillar and extrafibrillar space of collagen fibrils can be visualized. Note that the continuity of crystal orientation along the long axis of the fibers is disrupted primarily during TEM sample processing that involved crushing and dispersing the collagen fibrils on TEM grids, resulting in short and randomly oriented fibers/crystallites. A segment of the fibril that did not mineralize (faint contrast) is also present. Scale bar: 100 nm.

FIG. 11 depicts representative TEM images of 450 nm thick sections of (panel a) Non-mineralized and (panel b) Mineralized constructs. For the visualization of pure collagen, non-mineralized constructs were stained as described in the materials and methods section, while the mineralized constructs were left unstained with z-contrast provided by the mineral. Bundles of heavily calcified filamentous structures can be noticed in mineralized construct as opposed to the bare collagen in non-mineralized constructs. Some regions in the mineralized construct had little to no mineral associated with the fibrils. Scale bar: 500 nm.

FIG. 12 panels a and b depict backscattered block face SEM images of mineralized collagen fibrils acquired from two different regions of interest, at 2 kV after focal ion beam (FIB) milling. Scale bar: 500 nm.

FIG. 13 depicts TEM images showing the progressive mineralization of 3D collagenous matrix from day 1 to day 3. Panel a is an unstained TEM image depicting partially mineralized fibrils at day 1, with numerous mineral clusters deposited onto the surface of collagen fibrils. The corresponding high magnification image is shown in panel b. Panels c and d depicts TEM images after 3 days of mineralization. Extensive needle-like apatite deposition was observed, both at the intra and extrafibrillar space. Scale bar: 500 nm.

FIG. 14a is a graph of cell numbers in non-mineralized, mineralized, and OIM-treated matrix over time. Proliferation rate of hMSCs encapsulated in mineralized hydrogels was comparable to that of cells embedded in non-mineralized and OIM treated hydrogels up to day 3 and was significantly higher than both groups on days 7 and 14 (*p<0.05, **p<0.01, ANOVA/Tukey). After 21 days, a sharp decrease in cell number was recorded in all groups, with a significant reduction observed in non-mineralized (*p<0.05 ANOVA/Tukey) and mineralized (**p<0.01 ANOVA/Tukey) hydrogels in comparison with OIM-treated samples (N=6).

FIG. 14b depicts representative fluorescent micrographs of live (blue) and dead (green) stained hMSCs embedded in non-mineralized (left panel), mineralized (middle panel) and OIM-treated (right panel) hydrogels after 7 days. Scale bar: 100 μm.

FIG. 15 depicts representative alizarin red staining images of non-mineralized, CaP, mOPN, Mineralized, and OIM constructs (panels from left to right) collected at days 1, 7, and 21 (panels from top to bottom). Images depict homogenous calcification in the mineralized constructs within 7 days in culture. A total of 5×10⁴ cells were encapsulated in collagen (1.5 mg/ml) hydrogels. For the CaP condition, cells were cultured in DMEM supplemented with 4.5 mM of CaCl₂) and 2.1 mM of K₂HPO₄. For the mOPN condition, DMEM supplemented with 100 μg/mL of mOPN was used; in OIM group, the cells were cultured in DMEM containing 100 nM Dexamethasone, 50 UM ascorbic acid and 10 mM B-glycerol phosphate. Non-mineralized constructs were cultured in non-supplemented complete DMEM medium. After 1, 7 and 21 of culture, Alizarin Red S (Sigma) staining was performed to assess the overall mineralization. In brief, after culture, samples were washed in PBS, fixed in 4% paraformaldehyde for 5 min and then stained using 2% (w/v) Alizarin red solution at pH 4.2 for 5 minutes. After repeated washing in distilled water to remove any unbound stain, the constructs were imaged in bright field mode. Non-mineralized constructs showed no signs of mineral deposition even after 21 days, whereas the constructs exposed to osteoinductive supplements showed mineral nodule formation after 21 days of culture. The constructs treated with CaP containing medium resulted in random deposition of mineral nodules, while treatment with mOPN alone did not induce any visible mineral deposition. Of note, intense red staining was detected in the mineralized construct at day 7, suggesting the presence of dense calcium phosphate deposits uniformly distributed throughout the matrix. Scale bar: 100 μm

FIG. 16 is a graph of collagen and mineral volume calculated from the 3D reconstructed image of cell-laden mineralized matrix. Serial slices of resin embedded mineralized constructs obtained at 60 nm intervals using SBF-SEM microscope was aligned and processed using AMIRA (Version 5) image analysis and reconstruction software. Subsequently, semi-automated segmentation of mineral and collagen was performed slice-by-slice, followed by volume rendering to compute the 3D volume occupied by the specific features of interest. It was found that approximately 47% of the collagenous matrix was covered by mineral.

FIG. 17 are graphical representations of the results from gene expression analyses for hMSCs in cell-laden hydrogels after 7, 14 and 21 days of culture, with and without matrix mineralization, and in comparison to cells cultured in medium supplemented with calcium and phosphate, or mOPN alone. Graphs representing the expression of RunX2 (top left panel), ALP (top middle panel), OCN (top right panel), DMP1 (bottom left panel), and PDPN (bottom right panel) are shown. Culture of hMSCs in CaP-containing medium alone did not induce a significant upregulation of osteogenic markers. While incubation in mOPN alone had a significant increase in mRNA levels for OCN at day 7 (p<0.01) and ALP at day 21 (p<0.0001).

FIG. 18a depicts immunofluorescence staining images of the expression of osteocalcin (OCN) and DAPI in hMSCs encapsulated within hydrogels. Top panels depict images at day 7 and lower panels depict images at day 21. Panels from left to right depict images of non-mineralized constructs, mineralized constructs, and of encapsulated cells treated with osteoinductive medium as the positive control.

FIG. 18b show graphical representations of OCN stain fluorescence intensity (left panel) and percentage of OCN-positive cells (right panel). In non-mineralized constructs, OCN expression was poorly detected at the early time point. However, prolonged culture for 21 days within the construct resulted in a slight increase in the OCN expression level. OIM and mineralized constructs, on the other hand readily exhibited intense OCN expression within 7 days of culture. Unlike mineralized constructs, that retained OCN expression level after 21 days, cells in OIM had a markedly reduced expression. Scale bar: 100 μm.

FIG. 19a depicts immunofluorescence images of pre-osteocytic PDPN marker expression in hMSCs. Top panels depict images at day 7 and bottom panels depict images at day 21. Panels from left to right depict images from non-mineralized, mineralized, and positive control groups. Scale bar: 100 μm.

FIG. 19b show graphical representations of PDPN stain fluorescence intensity (left panel) and percentage of PDPN-positive cells (right panel). A time dependent increase in the expression of PDPN was noticed in non-mineralized and positive control groups, whereas the cells in the mineralized constructs showed a high PDPN expression even at the early time point of day 7.

FIG. 20 depicts fluorescence images of GFP-expressing hUVECS in co-culture with hMSCs (4:1 ratio) encapsulated in non-mineralized (left panel) and mineralized (right panel) hydrogels after 7 days in culture. Dense capillary-like networks are visible after 3 days of matrix mineralization, and mineralization had negligible effects on cell and network morphology. Scale bar: 500 μm.

FIG. 21 are representative immunofluorescence images depicting pericyte-supported endothelial network formation in non-mineralized (upper panels) and mineralized (lower panels) constructs. hMSCs grown in close contact with the hUVECs showed high expression of αSMA, a marker indicative of the pericytic differentiation of hMSCs. Panels from left to right depicts immunofluorescence images of F-actin, αSMA, DAPI, and of all three markers (Merged). Scale bar: 100 μm.

FIG. 22 graphically represents the results of the quantification of percentage vessel area, total number of vessel junctions, total vessel length and average vessel length as determined by Angiotool software (panels from left to right). A reduction in vessel parameters suggest that the mineralization process significantly affected vasculogenesis in-vitro (*p<0.05). A significant increase of all of the parameters for vasculature formation was found for mineralized samples after implantation in-vivo.

FIG. 23 panel a depicts a representative single block face SEM image illustrating cell morphology consistent with endothelial lumen formation within the mineralized hydrogels. Panel b depicts a high magnification image (black square in panel a) suggesting the presence of intracellular vacuoles within the cells, that appear to fuse to form lumen. Cement-line like hypermineralized boundaries can also be noted around the endothelial a lumen. Scale bar: 20 μm.

FIG. 24 depicts Alizarin red staining images for collagen hydrogels mineralized with increasing concentrations of Ca²⁺ and PO₄³⁻ on day 1 (panels a, d, g), day 3 (panels b, e, h), day 7 (panels c, f, i). Panels a-c depicts images of samples that were treated with 1.125 mM Ca²⁺ and 0.525 mM PO₄³⁻ daily for 3 days and analyzed on days (panel a) 1, (panel b) 3 and (panel c) 7. Virtually no red staining is present even after 7 days. Panels d-f depicts images of samples treated with 2.25 mM Ca²⁺ and 1.05 mM PO₄³⁻. The same pattern of negligible mineralization is seen. Panels g-i depicts images from samples that were treated with 4.5 a mM Ca²⁺ and 2.1 mM PO₄³⁻. A marked increase in Alizarin Red staining is seen on (panel h) day 3, and the red hue is maintained up to day 7 (panel i) (N=6).

FIG. 25 panels a-f depicts SEM micrographs showing representative (panels a, d) non-mineralized, (panels b, e) mineralized and (panels c, f) in OIM-treated collagen hydrogels after 3 days in lower magnification (panels a-c) and higher magnification (panels d-f). Panels g-i are graphical representations of the respective elemental composition of non-mineralized (panel g), mineralized (panel h), and OIM-treated (panel i) samples. Both non-mineralized (g) and OIM-treated samples (i) had only minor Ca and P peaks, while high peaks for both Ca and P were present in the mineralized samples (h). (N=4)

FIG. 26 depicts Selective Area Electron Diffraction (SAED) analysis images of (panel a) native bone, (panel b) mineralized collagen hydrogels after 3 days, and (panel c) osteoblast-secreted mineralized matrix [Adapted from Boonrungsiman et al.³⁶ with permission]. Despite the different intensities, the typical (002) plane and overlapping arcs for the (112), (211), and (300) are seen in all three samples, which is suggestive of the crystalline nature of the hydroxyapatite in all systems.

FIG. 27 panels a-f are graphical representations of the results from the analysis of nanoindentation elastic modulus of non-mineralized and mineralized collagen fibrils performed in water (panels a-c) and in air (panels d-f) using an atomic force microscope (Nanoscope 8 atomic force microscope, J scanner, Bruker). Representative load-displacement curves on (panel a) non-mineralized and (panel b) mineralized collagen fibrils, measured in water are shown. The loading curve is represented in red and the unloading curve is represented in blue. The dotted line corresponds to the indentation on fibrils, while the indentation on the adjacent mica substrate is denoted by the solid line. Inset shows the representative AFM topographic image of individual fibrils fixed onto freshly cleaved muscovite mica disc, with the color scale on the right corresponding to the height. Panel c is a graph showing the average elastic modulus in non-mineralized versus mineralized fibrils, calculated by fitting the Hertz model to the loading curves. An increase of over 1000-fold was recorded in hydrated mineralized fibrils versus non-mineralized controls (****p<0.0001, Student's t-test). Corresponding load-displacement curves and average modulus of non-mineralized and mineralized fibrils in air are shown in panels d, e and f, respectively. The nanoscale elastic moduli values in air followed the same trend, with a substantial increase in stiffness in mineralized fibrils compared to its non-mineralized counterparts (**** p<0.0001, Student's t-test). In all cases, the maximum penetration depth was set to ˜15% of the fibril thickness. In panels b-e the measurements were made using aluminum-coated, silicon microsphere tip of radius, 10 nm, resonance frequency of 300 KHz and a nominal spring constant of 26 N/m whereas for non-mineralized collagen fibril in panel a, indentation was performed using triangular gold-coated silicon nitride tip of 30 nm radius, 65 kHz resonance frequency and 0.35 N/m nominal spring constant. Measurements were performed on 5 to 10 different locations from at least 3 fibrils per sample, with a total of 3 samples per condition. Panel g is a graphical representation of the bulk elasticity (shear storage moduli) of mineralized and non-mineralized hydrogels. The bulk elasticity (shear storage moduli) of the mineralized hydrogels was significantly higher compared to their non-mineralized controls (*p<0.05, Student's t-test). An oscillatory rheometer fitted with an 8 mm parallel plate geometry was used to measure the storage modulus and loss modulus in frequency sweep mode at 1 Hz frequency and 1% strain (N=4).

FIG. 28 panel a depicts TEM images illustrating the alignment and banding pattern of collagen fibrils used for cell and mineralization studies. Panel b is a frequency plot graph of fibril diameter from a total of 96 measurements (N=4). The fibrils diameter was typically in the range of 60-120 nm, with higher frequencies found for 60, 100 and 120 nm diameter fibrils.

FIG. 29a depicts immunofluorescence images representative of the expression of pre-osteocytic marker, DMP1 (first and fourth columns from the left) in hMSCs encapsulated within non-mineralized (top row), mineralized (middle row) and OIM constructs (bottom row). Immunofluorescence images of cell nuclei stained with DAPI are shown in the second and fifth columns from the left. Merged images are shown in the third and sixth columns from the left. The first three columns from the left depict images at day 7 and the remaining columns depict images collected at day 21. Cells were immunolabelled with mouse anti-DMP1 antibody, followed by staining with secondary fluorochrome, AlexaFluor 647-conjugated goat anti-mouse IgG antibody. No DMP1 expression was detected in hMSCs at an early time-point of day 7, irrespective of the conditions tested. Conversely, the majority of the nuclei were stained positive for DMP1 in mineralized constructs on day 21 (white arrows) compared to faint nuclear staining in the non-mineralized group. Note the minimal expression of DMP1 in OIM treated group. Non-specific background labeling was noticed in all three constructs after extended culture for 21 days. DAPI is stained in blue.

FIG. 29b upper panel is a graph of the mean fluorescent intensity of DMP1 in stained non-mineralized, mineralized, and OIM constructs. The intensity was not statistically different between the mineralized and OIM constructs, both on day 14 and 21. The lower panel is a graph representing the percentage of cells expressing DMP1 after 7, 14 and 21 days. The highest fraction of DMP1-positive cells was recorded in the mineralized groups at day 14 and 21, with a statistically significant difference with respect to OIM at day 21. (*p<0.05; ** p<0.01, *p<0.001; **** p<0.0001, by ANOVA test; Data represented as mean±SD; (N=3); Scale bar: 100 μm).

FIG. 30 is a time lapse imaging sequence for hMSCs encapsulated in a mineralized collagen hydrogel (N=3) on day 7. Panels a-j depicts images collected between 0 and 90 mins in 10 min increments. Panels k-m depict zoomed-in images of cells at time points 0, 50, and 90 mins. Images were acquired at 20× magnification for 90 min at 10-min intervals with Zeiss airyscan 880 microscope and processed with ImageJ software. Cells were stained using a cell tracker (deep red cell tracker, Invitrogen, 1 μg/ml) and images were generated both in fluorescence and transmission modes, and then merged. A single cell is shown with a white arrow to illustrate cell movement within the mineralized matrix. On the image in (panel a), the right and lower edges of the cell are bordered with a tangential dotted line to mark the starting point of a cell at time point 0; the lines are not moved across the time points for easier reference. The movement of the cell away from the reference lines facilitates visualization of the movement path of the cell from 0 to 90 min (panels a-j). A zoomed-in view of a single cell at time points 0 (panel k), 50 (panel l) and 90 min (panel m) show the morphological changes to the cell cytoplasm and changes in the formation of cell processes that extend and contract in different directions over time. In (panel I) multiple narrow processes are shown in a cell (yellow arrow), and (panel m) after 90 min, these processes have either disappeared or moved to another location (green arrows). Scale bar: 50 μm.

FIG. 31 panels a-I depict images of stained with dual calcium indicators (cell permeant Fluo-4AM dye (494/506 nm) and Rhod-5N dye (551/576 nm)), which were used to distinguish intracellular and extracellular Ca2⁺, respectively. Images of mineralized samples at day 7 (panels a-f) and at day 14 (panels d-f), and images of OIM-treated samples at day 7 (panel g-i) and day 14 (panel j-I) are shown. Panels from left to right show images of samples stained with Fluo-4AM dye, Rhod-5N dye, and that are merged. After 7 and 14 days of culture in mineralized and OIM treated collagen matrix, hMSCs were loaded with 5 uM of Fluo-4 AM for 2 hours. Post incubation, excess Fluo-4 AM was rinsed off, followed by loading of 5 uM of Rhod-5N and the samples were subsequently imaged live to visualize the increase in fluorescence intensity upon binding to Ca2⁺ using LSM 880 Laser scanning Confocal microscope (N=3). Cells cultured in mineralized collagen (panels a-c) had a two-fold increase in cytosolic calcium (green) on day 7, compared to those cultured in OIM (*p<0.05). For OIM, these levels were only attained on day 14 (panels j-I). Extracellular calcium (red) appears to be distributed throughout the matrix since day 7 in mineralized collagen (panels b-c, e-f), with higher intensity spots surrounding cells on day 14, possibly due to cell-secreted minerals. OIM samples had very little extracellular calcium on day 7 (h-i) and only localized calcium deposits in the pericellular regions on day 14 (panels k-l). Scale bar: 50 μm. Panel m is a graphical representation of Fluo-4 intensity.

FIG. 32 depicts Alizarin red staining images showing the time-controlled mineralization of collagen hydrogel. Panel a depicts images of samples that were treated with mineralization media for 3 continuous days then maintained in DMEM medium for the remainder of the experiment. Panel b depicts images of samples that were subjected to the 7-day 2 step-mineralization procedure. Partial calcification of the collagen matrices was induced by exposing the samples to mineralizing medium (cell-culture medium with supersaturated Ca and P ions stabilized with mOPN) for 2 days. Next, the medium was replaced with standard DMEM without additional Ca and P or mOPN, to stop mineralization until day 4. Note the faint red staining till day 5 due to limited mineralization of the matrix. On day 5, samples were cultured with the Ca, P and mOPN rich medium again, thus resuming the mineralization process and completing matrix calcification, as visualized from the intense red staining at later time-points. After each time-point, the samples were fixed, stained with 2% Alizarin red S and imaged under bright field mode using EVOS FL Auto 2 Imaging System (N=6). Scale bar: 400 μm.

FIG. 33 depicts images of H&E stained (panels a, b) and DMP1 stained (panels c, d) hydrogels that are non-mineralized (panels a, c) and mineralized (panels b, d). H&E images of non-mineralized (panel a) and mineralized (panel b) hydrogels show erythrocyte filled capillaries (yellow arrow head) within the constructs after 3 weeks of implantation in nude mice. Scale bar: 500 μm. Expression of DMP1 was visible in (panel c) non-mineralized and (panel d) mineralized constructs implanted with pre-formed vasculature after 7 days of culture in-vitro, followed by 7 days in-vivo. DMP1-expressing cells (yellow arrows), which are evident even in mineralized constructs some of which are present near cross-sectioned luminal structures that are consistent with the appearance of vasculature capillaries (yellow arrowheads, unstained), demonstrate osteoblast formation in conjointly with vasculature in these mineralized samples. Scale bar: 100 μm (N=4).

### DETAILED DESCRIPTION

It will be readily understood that the embodiments, as generally described herein, are exemplary. The following more detailed description of various embodiments is not intended to limit the scope of the present disclosure, but is merely representative of various embodiments. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified.

Disclosed herein are cell-laden matrix compositions. The compositions may include:
(a) a mineralizing solution, the mineralizing solution being supersaturated with respect to one or more crystallizable metals and having a pH from about 6.0 to about 8.0;
(b) a buffering agent having a pH buffering range of about 6.0 to about 8.0;
(c) living cells;
(d) a scaffold;
(e) a basal medium for supporting the growth of the living cells; and
(f) a nucleation inhibitor.

In particular, methods of culturing biomimetic bone tissue are disclosed herein. The methods may include providing a cell culture medium including living cells and a basal medium. The methods may further include providing a mineralizing solution containing a supersaturated solution with respect to ionic calcium and ionic phosphorous and a nucleation inhibitor. The methods may include providing a collagen scaffold and exposing the collagen scaffold to the cell culture medium to associate living cells with the collagen scaffold. Next, the collagen scaffold and associated living cells may then be exposed to the mineralizing solution to achieve a selected mineralization level.

Also disclosed herein are kits containing compositions disclosed herein or portions thereof. The kits may be useful in performing the methods disclosed herein. For example, the kits may include a container with contents that include: (i) a mineralizing solution, the mineralizing solution being supersaturated with respect to one or more crystallizable metals; (ii) a basal medium for supporting the growth of living cells; (iii) a nucleation inhibitor; and (iv) a buffering agent having a pH buffering range of about 6.0 to about 8.0. In other variations, the kits may include a first container with contents including (i) a basal medium for supporting the growth of living cells; and (ii) a buffering agent having a pH buffering range of about 6.0 to about 8.0. The kits may further include a second container with contents including: (i) a mineralizing solution, the mineralizing solution being supersaturated with respect to one or more crystallizable metals and having a pH from about 6.0 to about 8.0; and (ii) a nucleation inhibitor. In particular embodiments, the mineralizing solution may include a supersaturated solution with respect to ionic calcium and ionic phosphorus. Likewise, in particular embodiments, the mineralizing solution may include a supersaturated solution with respect to either ionic calcium or ionic phosphorus and the kit includes an additional container with contents comprising a supersaturated solution of the other of ionic calcium or ionic phosphorous.

The mineralized matrix comprising living cells disclosed herein may be used as models or replacements for mineralized natural tissues, including bone, dentine, and calcified cartilage.

Particular embodiments provide a method for treating a bone defect, the methods comprising applying to an area of bone in need thereof, a mineralized matrix comprising living cells, as disclosed herein.

In some embodiments herein, the living cells contained in the matrix may be cells collected from a healthy bone, preferably in the same subject having a bone defect into which the matrix comprising living cells will be applied. The living cells will be collected from healthy bone marrow in some embodiments.

Also provided is a method of selectively mineralizing tissue-graftable bone marrow cells to a tissue-graft site, such as a human or animal subject, the method comprising the steps of: providing a mineralization solution comprising a supersaturated solution with respect to ionic calcium and ionic phosphorus and a nucleation inhibitor; collecting endogenous bone marrow cells from a healthy bone of a subject, the subject having a tissue-graft site; exposing the endogenous bone marrow cells to the mineralization solution for a period to achieve a selected mineralization level and form a mineralized bone marrow graft; and, applying the mineralized bone marrow graft to the tissue-graft site.

Also provided is a method of selectively mineralizing a cellularized matrix, the method comprising the steps of: providing two or more matrix modules, each of the matrix modules having a module periphery and having completed a curing process; providing a cellularization solution containing living cells; providing a mineralization solution comprising a supersaturated solution with respect to one or more crystallizable metals and a nucleation inhibitor; exposing the matrix modules to the cellularization solution for a period to deposit the living cells on the module peripheries of the matrix modules; exposing the matrix modules and living cells to the mineralization solution to achieve a selected mineralization level; combining the matrix modules such that the module peripheries of two or more matrix modules make contact to form a cellularized matrix. In some embodiments, the matrix modules may be formed as micrometer-scale or millimeter-scale volumes. In other embodiments, the matrix modules may be composed of hydrogel material. In further embodiments, the matrix modules may be composed of supermolecular hydrogel material (Sun, N. et al. Carbohydr Polym. 172:49-59). In some embodiments, the curing process increases the flexural strength of the matrix modules, preventing cellular migration from the peripheries of the matrix modules to their interiors. In various embodiments, the curing process may include ionic setting, photo cross-linking, temperature-based setting, “click chemistry,” cross-linking, or freezing. In some embodiments, the cellularization solution contains living cells of the types described herein. In other embodiments, the cellularization solution contains a sufficient concentration of living cells to deposit the living cells across the entirety of matrix module peripheries. In further embodiments, the mineralization solution is composed as described herein, and the period of exposing the matrix modules and living cells to the mineralization solution is determined by the user to achieve a selected level of mineralization. In some embodiments, two or more matrix modules are combined such that the matrix module peripheries make contact, thereby creating cell migration pathways along the mutually contacting matrix module peripheries that extend through the cellularized matrix.

The term “bone marrow” refers to the areas of natural bone containing both hematopoietic stem cells (HSCs) and nonhematopoietic cells. HSCs give rise to all types of mature blood cells, whereas the nonhematopoietic component is composed of osteoblasts/osteoclasts, endothelial cells, endothelial progenitor cells, T lymphocytes, macrophages, mast cells, stromal fibroblasts and mesenchymal stem cells. All of these cells contribute to the formation of specialized ‘niches’, which are close to the marrow vasculature (‘vascular niche’) or to the endosteum ‘endosteal niche, both of which are important in the structure and function of the bone marrow.

In the embodiments disclosed herein, the living cells may be mammalian cells. Exemplary types of mammalian cells include bone-derived cells, mesenchymal stem cells, hematopoietic stem cells, osteoblasts, progenitor cells, multipotent progenitor cells, common myeloid progenitor cells, common lymphoid progenitor cells, megakaryocyte-erythroid progenitor cells, adipocytes, macrophages, granulocyte/macrophage progenitor cells, endothelial cells, osteoblast precursor cells, osteolineage cells, pericytes, chondrocyte precursor cells, mesenchymal stem, progenitor cells, mesenchymal stromal progenitor cells, or combinations thereof. In particular, non-limiting examples of mammalian cells that may be used in the embodiment disclosed herein include common myeloid progenitor cells, common lymphoid progenitor cells, adipocytes, macrophages, granulocyte/macrophage progenitor cells, endothelial cells, osteoblast precursor cells, osteolineage cells, pericytes, chondrocyte precursor cells, mesenchymal stem and progenitor cells, mesenchymal stromal progenitor cells, reticulocytes, hemocytoblasts, proerythroblasts, erythroblasts, normoblasts, polychromatic erythroblasts, myeloblasts, progranulocytes, lymphoblasts, monoblasts, promonocytes, monocytes, megakaryoblasts, megakaryocytes, megakaryocyte progenitor cells, erythrocyte progenitor cells, megakaryocyte-erythrocyte progenitor cells, pro-natural killer cells, pro-B cells, pre-B cells, myeloid stem cells, myeloblasts, promyelocytes, myelocytes, basophilic myelocytes, basophilic meta-myelocytes, metamyelocytes, band forms, eosinophilic myelocytes, eosinophilic metamyelocytes, neutrophilic myelocytes, neutrophilic meta-myelocytes, fibrocytes, neutrophilic band cells, cells derived from hemopoiesis, leukopoiesis, erythropoiesis, granulopoiesis, lymphopoiesis, or combinations of any of the foregoing. The living cells may be at a concentration from about 1×10⁵ cells/mL to about 10×10⁵ cells/mL in the matrix.

In some embodiments, the living cells in the matrix will comprise “immature cells” or “immature bone cells”, which refers to any cell type that is naturally found in a hematopoietic/vascular/stem cell niche of bone or in an endosteal niche of bone and has not reached a natural state of maturation, including those at a primary or intermediate levels of maturation. Examples include mesenchymal stem cells, hematopoietic stem cells, osteoblasts, and progenitor cells, multipotent progenitor cells, common myeloid progenitor cells (CMPs), common lymphoid progenitor (CLP) cells, megakaryocyte-erythroid progenitor cells (MEPs), adipocytes, macrophages, granulocyte/macrophage progenitor (GMP) cells, endothelial cells (ECs), osteoblast precursor cells, osteolineage cells, pericytes, chondrocyte precursor cells, mesenchymal stem and progenitor cells or mesenchymal stromal progenitor cells (MSPCs).

These immature cells also include those undergoing developmental pathways in hematopoietic, mesenchymal, bone and vascular lineages including but not limited to, common myeloid progenitor cells (CMPs), common lymphoid progenitor (CLP) cells, megakaryocyte-erythroid progenitor cells (MEPs), adipocytes, macrophages, granulocyte/macrophage progenitor (GMP) cells, endothelial cells (ECs), osteoblast precursor cells, osteolineage cells, pericytes, chondrocyte precursor cells, mesenchymal stem and progenitor cells or mesenchymal stromal progenitor cells (MSPCs), hemopoiesis, leukopoiesis, erythropoiesis, granulopoiesis, lymphopoiesis, etc. Specific cell examples include, but are not limited to, reticulocytes, hemocytoblasts, proerythroblasts, erythroblasts, normoblasts, polychromatic erythroblasts, myeloblasts, progranulocytes, lymphoblasts, monoblasts, promonocytes, monocytes, megakaryoblasts, megakaryocytes, megakaryocyte progenitor cells, erythrocyte progenitor cells, megakaryocyte-erythrocyte progenitor cells, pro-natural killer cells, pro-B cells, pre-B cells, common myeloid progenitor cells, common lymphoid progenitor cells, myeloid stem cells, myeloblasts, promyelocytes, myelocytes, basophilic myelocytes, basophilic meta-myelocytes, metamyelocytes, band forms, eosinophilic myelocytes, eosinophilic meta-myelocytes, neutrophilic myelocytes, neutrophilic meta-myelocytes, fibrocytes, and neutrophilic band cells.

In some embodiments, the matrix contains cells found in the hematopoietic niche of normal bone, such as stem cells. Cells found contained in this domain may include those selected from the group of hematopoietic stem cells (HSCs), long-term hematopoietic stem cells (LT-HSCs), short-term hematopoietic stem cells (ST-HSCs), multipotent progenitor cells, common myeloid progenitor cells (CMPs), common lymphoid progenitor (CLP) cells, megakaryocyte-erythroid progenitor cells (MEPs), adipocytes, macrophages, granulocyte/macrophage progenitor (GMP) cells, endothelial cells (ECs), osteoblast precursor cells, osteolineage cells, pericytes, chondrocyte precursor cells, mesenchymal stem and progenitor cells or mesenchymal stromal progenitor cells (MSPCs), and other specialized marrow stromal populations such as CXCL12-abundant reticular (CAR) cells.

It should be understood that a “basal medium” referred to herein indicates a biologically acceptable medium or growth medium that facilitates maintenance of the living cells in a given matrix. In some embodiments, the basal medium is an aqueous medium comprising nutrients needed for cell growth and reproduction. Basal media may also contain additional agents, not limited to antibiotic agents, antifungal agents, antiviral agents, buffers, anticoagulants, vitamins, salts, minerals, amino acids, nucleic acids, ribonucleic acids, fatty acids, lipids, O₂ and/or CO₂ gases, carbohydrates, serum proteins, cofactors, growth factors, cytokines, enzymes, hormones, signaling substances, antibodies, among others, or combinations thereof. Non-limiting examples of basal medium that may be used include Modified Dulbecco's Medium (DMEM), phosphate buffered saline (PBS or DPBS), sodium bicarbonate buffers, RPMI or RPMI1640, Eagle's essential medium (EEM), EMM medium, Hanks' salts medium (HMEM), Hank's Balanced Salt Solution (HBSS), Earle's Balanced Salt Solution (EBSS), Iscove's modified Dulbecco's Medium (IMDM), Osteoblast Medium (ObM), fetal bovine serum (FBS), or combinations thereof.

In particular embodiments, such as bone-related embodiments, the scaffold may include collagen, such as a type 1 collagen matrix. For example, the type 1 collagen matrix may be prepared by reconstituting acid solubilized type 1 collagen. Preferably, the collagen may be at a concentration of about 0.5 mg/mL to about 5.0 mg/mL; however, concentrations from about 0.1 mg/mL to about 50 or 100.0 mg/ml are also possible. Other examples of collagen include: collagen type II, collagen type III, collagen type IV, collagen type V, collagen type VI, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, and collagen type XX, or a combination thereof. “Type I collagen” or “Type 1 collagen” refers to the fibrillar-type collagen that is the most abundant form of human collagen and the key structural composition of several tissues. “Fibrillogenesis” refers to the development of fine fibrils normally present in collagen fibers. Collagen cross-linking in native collagen contributes to fibrillogenesis, matrix stability, and elasticity. The term “scaffold” herein refers to a three-dimensional structure or matrix composed of natural or synthetic polymer fibers and biologically acceptable materials, such as useful in forming an environment conducive to the stimulation of bone cell growth and bone construction or repair.

For bone-related embodiments, the collagen needs to be fibrillated. This can be done before or after introduction of the living cells to the scaffold. There can be benefits to performing fibrillation in the presence of the living cells. To achieve fibrillation in this manner, the pH of the cell-laden matrix should be about 6.0 to about 8.0 and the temperature maintained from about 34° C. to about 40° C. until the collagen chains undergo fibrillogenesis. For non-collagen scaffolds, such as for non-bone-related embodiments, the scaffolds should also be cured, such as by gelation.

Hydrogels may be used in providing the matrices for use in the embodiments disclosed herein. The term “hydrogel” as used herein refers to a gel comprising a cross-linked network of water-soluble polymers capable of forming a matrix mimicking a natural extracellular matrix and supporting the biological materials and activities of interest to the present studies. Commercially available hydrogels include the MATRIGEL™ matrix (available from Corning Inc., Tewksbury, Massachusetts); poly [2-(methacryloyloxy)ethyl dimethyl (3-sulfopropyl) ammonium] (PMEDSAH) hydrogels or copolymers or blends thereof; glycoprotein hydrogels, such as fibronectin hydrogels and laminin hydrogels; protein hydrogels, such as those derived from collagen, albumin, fibrin, or silk proteins; polysaccharide hydrogels, such as those derived from glucan, hyaluronic acid, chitosan, agarose, and alginate; synthetic hydrogels composed of synthetic monomers such as those selected from the group of poly(ethylene glycol) (PEG), poly(vinyl alcohol) (PVA), poly(ethylene oxide) (PEO), poly(acrylic acid) (PAA), poly(hydroxyethyl methacrylate) (PHEMA), poly(methacrylic acid) (PMMA), polypropylene fumarate-co-ethylene glycol (P (PF-cop-EG)), poly(acrylamide) (PAAm), and poly N-isopropylacrylamide (PNIPAAm); and hybrid synthetic-biologic hydrogels having combined monomers of synthetic and biological materials, such as PEG-peptide hydrogels, including PEG-fibrinogen hydrogels. The term “hydrogel”, as used herein, is understood to include a single type of hydrogel material, such as one of the individually listed hydrogel materials above, or a mixture or combination of two or more individual hydrogel materials, such as a combination of the MATRIGEL™ matrix with collagen and/or a fibronectin hydrogel.

A “cross-linking agent” herein refers to an agent that facilitates the cross-linking of polymer chains to form a matrix of cross-linked polymer chains, such as a collagen or hydrogel. For various matrices, the cross-linking agent will vary by the polymer chains involved. Polyvinyl alcohol hydrogels may be cross-linked using sodium borate/boric acid as a cross-linking agent. Glyoxal may be used as a cross-linking agent for polyvinyl alcohol, starch, cellulose, or protein and gelatin hydrogels. Other hydrogel/cross-linking agent combinations include: polyethylene hydrogel/silane, agarose and chitosan hydrogels/oxidized dextrins, chitosan/glutaraldehyde, guar gum hydrogels/epichlorhydrin, Gellan gum hydrogels/endogen polyamine spermidine, glycol chitosan hydrogels/oxidized alginate, hydroxamated alginates/zinc, alginate beads/zinc, scleroglucan/Borax, poly(acrylic-co-vinylsulfonic) acid hydrogels/ethylene glycol dimethacrylate (EDGMA), polyacrylamide hydrogels/N,N′-methylenebisacrylamide, and polyacrylamide/guar gum graft copolymer hydrodgels/glutaraldehyde. A “cross-linking treatment” herein refers to any method of subjecting a group of non-linked polymers to an agent, force, or set of conditions that facilitate cross-linking of the polymers to form a desired matrix. Examples of cross-linking treatments include regimens of photochemical cross-linking or radiation-induced cross-linking.

In some embodiments herein, the matrix comprises one or more acidic polymers selected from the group of polyacrylic acid, polymethacrylic acid, sulfonated polymer, phosphorylated proteins or peptides, phosphorylated synthetic polymers, sulfated polysaccharides, sulfated glycoproteins, polyaspartic acid, polyglutamic acid, polyaspartate, polyvinyl phosphate, and polyvinyl phospbonate, or combinations thereof.

In additional embodiments, the final matrix may further comprise a natural biological stimulating factor, such as, but not limited to, peptide signaling molecules, bone morphogenetic proteins (BMPs), transforming growth factor beta (TGF-β), insulin-like growth factors I and II (IGF-I and IGF-II), platelet derived growth factor (PDGF), vascular endothelial growth factor-A (VEGF) and basic and acidic fibroblast growth factor (bFGF and aFGF).

In some embodiments, the final matrix further comprises microvascular fragments. The term “microvascular fragments” refers to fragments of adipose microvasculature generally collected and chopped to a fine size, followed by digestion with collagenase, usually with agitation, followed by centrifugation and separation using a series of filters of defined pore size. In some examples, larger pieces may be removed using a 200 μm nylon filter and individual cells may be removed using a 20 μm filter membrane. The microvascular fragments are also known as “processed microvascular tissue” or “adipose tissue-derived microvascular fragments (ad-MVF).”

In some embodiments, the final mineralized matrix comprises mesenchymal stem cells and microvascular fragments.

Examples of crystallizable metals include alkali metals, earth alkali metals, or both. In particular embodiments, such as bone-related embodiments, the crystallizable metals include ionic calcium and ionic phosphorus, such as from about 3.0 mM to about 6.0 mM (including about 4.0 mM to about 5.0 mM) of ionic calcium and such as about 1.5 mM to about 3.0 mM (including about 1.8 mM to about 2.5 mM) of ionic phosphorus.

Calcium-containing ionic materials that can be used as calcium “drug” or as the calcium source in a “mineralizing solution” are calcium chloride (anhydrous: CaCl₂, monohydrate: CaCl₂·H₂O, dihydrate: CaCl₂·2H₂O, or hexahydrate: CaCl₂·6H₂O), dicalcium phosphate dehydrate (CaHPO₄·2H₂O; DCPD), calcium sulphate dehydrate (CaSO₄·2H₂O; CSD), calcium sulphate hemihydrate (CaSO₄·½H₂O; CSH), calcium sulphate (CaSO₄), calcium acetate (anhydrous: Ca(C₂H₃O₂)₂, monohydrate: Ca(C₂H₃O₂)₂·H₂O, or dihydrate Ca(C₂H₃O₂)₂·2H₂O), calcium citrate (Ca3 (C₆HsO₇)·4H₂O), calcium fumarate (CaC₄H₂O 4·3H₂O), calcium glycerophosphate (CaC₃H₅(OH₂)PO₄), calcium lactate (Ca(C₃HsO₃)₂·5H₂O), calcium malate (dl-malate: CaC₄H₄O₅-3H₂O, 1-malate: CaC₄H₄O₅·2H₂O, or malate dihydrogen: Ca(HC₄H₄O₅)₂·6H₂O), calcium maleate (CaC₄H2O₄·H₂O), calcium malonate (CaC₃H₂O₄·4H₂O), calcium oxalate (CaC₂O₄), calcium oxalate hydrate (CaC₂O₄·H₂O), calcium salicylate, (Ca(C₇H₅O₃)₂·2H₂O), calcium succinate (CaC₄H₆O₄·3H₂O), calcium tartrate (d-tartrate: CaC₄H₄O₆·4H₂O; dl-tartrate: CaC₄H₄O₆·4H₂O; mesotartrate: CaC₄H₄O₆·3H₂O), and calcium valerate (Ca(C₅H₉O₂)₂).

Phosphate-containing ionic materials that can be used as a phosphate source in a “mineralizing solution” include dicalcium phosphate dehydrate (DCPD), sodium phosphate (Na₂HPO₄, NaH₂PO₄ or a mixture thereof; non-hydrated or hydrated species like Na₂HPO₄·2H₂O, Na₂HPO₄·7H₂O, Na2HPO₄·12H₂O, NaH₂PO₄·H₂O, NaH₂PO₄·2H₂O), calcium glycerophosphate (CaC₃H₅(OH₂)PO4), potassium orthophosphate (K₃PO₄), dihydrogen potassium orthophos-phate (KH₂PO₄), monohydrogen potassium orthophosphate (K₂HPO₄), and sodium orthophosphate (Na₃PO₄·10H₂O and Na₃PO₄·12H₂O).

The “mineralizing solution” or “mineralizing solutions” used herein refer to solutions, preferably aqueous solutions that provide an ionic source for a desired mineralization of a specified matrix herein. In some embodiments, the mineralizing solution comprises a calcium ion solution. In other embodiments, the mineralizing solution comprises a phosphate ion solution. In further embodiments, the mineralizing solution may comprise ions of magnesium, sodium, potassium, carbonate, iron, barium, boron, strontium, copper, and/or zinc.

In some embodiments, the mineralizing solution is one containing one or more sources of ionic minerals selected from the group of calcium phosphate, calcium carbonate, hydroxyapatite, strontium carbonate, barium carbonate, and calcium sulfate, strontium sulfate, calcium oxalate, magnesium-bearing calcium carbonate, and magnesium-bearing calcium phosphate.

In some embodiments, the mineralizing solution is a calcifying solution. In some embodiments, the calcifying solution comprises calcium and phosphate ions. In some embodiments the calcifying solution comprises a calcium salt selected from the group of calcium phosphate, calcium carbonate, calcium chloride (including those selected from the group of anhydrous CaCl₂, CaCl₂·H₂O, CaCl₂·2H₂O, and CaCl₂·6H₂O), calcium citrate, calcium glubionate, calcium gluconate, calcium acetate, and calcium lactate.

In other embodiments, the mineralizing solution is prepared using hydroxyapatite, octacalcium phosphate, tricalcium phosphate, carbonated hydroxyapatite, fluorinated hydroxyapatite, brushite, magnesium-containing hydroxyapatite, dicalcium phosphate dihydrate, and amorphous calcium phosphate.

The term “nucleation inhibitor” refers to an agent that inhibits crystal nucleation or crystal growth, or reduces the rate of crystal nucleation or growth, in solution. In particular embodiments, such as bone-related embodiments, the nucleation inhibitor preferably inhibits nucleation or precipitation of hydroxyapatite. The nucleation inhibitor may be a non-collagen protein (NCP) or function as a NCP analog, such as an acidic NCP or NCP analog. In particular embodiments, the nucleation inhibitor is only an NCP, such as only an acidic NCP. For example, the nucleation inhibitor may include Osteopontin, Osteocalcin, Osteonectin, bone sialoprotein, dentine phosphoryn, dentin matrix protein 1, dentin sialophosphoprotein (DSPP), matrix extracellular phosphoglycoprotein, chondrocalcin, proline-rich proteins such as Proline-rich protein 1, Proline-rich protein 2, and Proline-rich protein3, PRP1-T1, PRP3-T1, Histatin 5, MG1, MG2, Asialo_MG2, Amylase, statherin, cystatin S, cystatin SN, Cystatin S1, fetuin, HSA, or combinations thereof. In the case of osteopontin, the osteopontin concentration may be from about 50 μg/mL to about 1000 μg/mL. In a supersaturated Ca and P solution about 50 μg/mL to about 150 μg/mL of osteopontin is preferred.

The term “NCP analog” refers to compounds or materials that mimic the natural activity of non-collagenous proteins in binding to scaffold fibers, such as in Type 1 collagen, and the formation of bone tissue. Examples include synthetic polymers/peptoids, such as those discussed by Chien et al., ACS Biomater. Sci. Eng. 2017, 3, 3469-3479., biomimetic polyelectrolyte and poly(amino) acid macromolecules that mimic the functional domains of natural NCPs can be employed in certain embodiments of the present invention (Stupp, S. I. et al., Science, 1997, 277:1242-1248; Girija, E. K. et al., J. Mater. Sci.: Mater. Med., 2004, 15:593-599). The NCP mimic may also be a poly(amino) acid polyelectrolyte (or polyanion), including carboxylic acid-containing polyelectrolytes such as, polyacrylic acid (PAA), substituted polymethacrylates (PMA), polysulfonates, phosphorylated proteins, peptides, polymers, sulfated glycoprotein, polyglutamic acid, polyaspartic acid, polyvinyl phosphates, polyvinyl phosphonates, acrylophosphonic acid, polyvinylphosphonic acid, polystrenephosphonic acid, diisopropyl vinyl phosphonate, 1-hydroxyethylidene-1,1-diphosphonic acid, 2-phosphonobutane-1,2,4-tricarboxylic acid and mixtures thereof. Poly(aspartic) acid and polyacrylic acid have been employed as biomimetic analogs of acidic non-collagenous proteins such as dentin matrix protein 1 (DMP1) (He, G. et al., Biochemistry, 2005, 44:16140-16148)⁶². for stabilizing and controlling the dimensions of amorphous phases in calcium carbonate and calcium phosphate precipitation systems (Olszta, M. J. et al., Connect Tissue Res., 2003, 44 (Suppl 1): 326-334; US Patent Application 2006/0204581)⁶⁸.

In particular embodiments, during the mineralization process, the pH of the mineralizing solution is preferably maintained from about 7.2 to about 7.6. The mineralization process may be for a sufficient time period to achieve the desired mineralization. One of the benefits of the methods and compositions disclosed herein is that significant mineralization can occur in an as few as three days. For example, in the examples discussed below, more mineralization occurred in three days than occurred under a comparative approach in twenty-one days. Additionally, mineralization can be stopped and started as needed using the methods and compositions disclosed herein. Exposing the cell-laden matrix to the mineralizing solution may be only about 1 minute to about 7 days or more, such as about 10 minutes to about 60 minutes or several weeks.

For research and testing purposes, in some embodiments of the present methods and compositions, the living cells comprised in the mineralized matrix are diseased cells. A “diseased cell” or a “diseased state cell” refers to a cell experiencing a pathologic, oncologic, or other disease challenge. Diseased cells for use in the models, designs, devices, and methods herein may be from any source, including disease cell lines or patient/donor samples. In some embodiments, such as disease models of bone marrow, both the endosteal and hematopoietic niches comprise diseased cells.

Diseased cells that may be included in this model include—but not limited to—leukemia [acute myeloid leukemia (AML), chronic myeloid leukemia (CML), atypical CML, chronic neutrophilic leukemia, acute lymphoblastic leukemia (ALL), etc.], multiple myeloma, smoldering myeloma, monoclonal gammopathy of undetermined significance, Non-Hodgkin lymphoma, Chronic lymphocytic leukemia (CLL), monoclonal B lymphocytosis, Hodgkin lymphoma, T-cell lymphoma, bone marrow failure syndromes, myelodysplastic syndrome (MDS), clonal hematopoiesis of indeterminate potential (CHIP), clonal cytopenias of undetermined significance (CCUS), aplastic anemia, and metastatic solid tumors that travel to the bone marrow (lung, breast, kidney, prostate, thyroid, etc.). It is understood that hemopoietic stem cells found in this domain may be quiescent or proliferating. It is understood that hemopoietic stem cells found in this domain may be quiescent or proliferating.

Diseased state cells can include, but are not limited to, macrocytes, polychromataphilic reticulocytes, aggregate reticulocytes, punctate reticulocytes, target cells, spherocytes, ovalocytes/elliptocytes, stromatocytes, sickle cells, acanthocytes, schistocytes, helmet cells, dacrocytes/teardrop cells, echinocytes/Burr cells, Pappenheimer bodies, Cabot ring cells, punctate basophilia/basophilic stippling cells, Heinz-Endrich bodies, codocytes/leptocytes, megaloblastic cells, hypochromic red blood cells, microcytic red blood cells, macrocytic red blood cells, knizocytes, degmacytes, fragmented red blood cells, Thalassemia red blood cells, Bite cell red blood cells, Hemoglobin C Crystal red blood cells.

The diseased state cells can also include cells of bone marrow cancers, including mature cancer cells, cancer induced angiogenesis, including, but not limited to, multiple myeloma cells and multiple myeloma precursor cells (cells exhibiting monoclonal gammopathy of unknown significance and smoldering myeloma cells), leukemic stem cells, leukemic blast cells, and leukemic promyelocytes.

A “diseased state” is an abnormal condition that negatively affects the state or function of at least part of a subject, whether or not symptoms have yet been manifested. A niche, domain, cell, or patient “subject to” a particular disease or malady refers to conditions in which the underlying basis for a future disease state are present (such as a genetic condition, pathogen, nutrient or biochemical deficiency, etc.), though symptoms have not yet been manifested.

Disease states that may be studied using the devices, designs, and methods herein include leukemias (including Acute Myelogenous Leukemia (AML), Chronic Myelogenous or Myeloid Leukemia (CML), Atypical CML, Acute Lymphoblastic Leukemia (ALL), Chronic Lymphocytic Leukemia (CLL), Chronic Neutrophilic Leukemia, Childhood Leukemia, Chronic Myelomonocytic Leukemia, Megakarocytic Leukemia, Chronic Myelogenous Leukemia, Juvenile Myelomonocytic Leukemia (JMML), Acute monocytic leukemia (AMOL), Atypical Chronic Myelogenous Leukemia, lymphoblastic and lymphocytic leukemias), Multiple Myeloma, Bone Marrow Failure Syndrome, clonal hematopoiesis of indeterminate potential (CHIP), clonal cytopenias of undetermined significance (CCUS), hemophagocytic lymphohistiocytosis, Wiskott-Aldrich syndrome, Bone Marrow Adiposity, aplastic anemia, Fanconi Anemia, Sickle Cell Anemia, Pure Red Cell Aplasia, myelodysplastic or myeloproliferative disorders/syndromes and neoplasms, Myelofibrosis, Paroxysmal Nocturnal Hemoglobinuria, Polycythemia Vera, Thrombocythemia, Thrombocytopenia, Thrombocytosis, and Thalassemia Major and Minor. The diseased state may also include cancers originating in bone, including osteosarcoma, chondrosarcoma, and Ewing's Sarcoma, as well as metastatic cancers including, but not limited to lymphomas (Hodgkin lymphomas, such as nodular sclerosing subtype, mixed-cellularity subtype, lymphocyte-rich subtype, or lymphocyte depleted subtype; and Non-Hodgkin, and T-cell Lymphomas), and cancers originating in other organs or tissues, including, but not limited to, the prostate (e.g. metastatic castration resistant prostate cancer), colon, breast (e.g. triple negative breast cancer), kidney (e.g. renal cell carcinoma), lung cancer (e.g. non-small cell lung cancer), and thyroid.

Diseased cells that may be included in these models include—but not limited to—leukemia [acute myeloid leukemia (AML), chronic myeloid leukemia (CML), atypical CML, chronic neutrophilic leukemia, acute lymphoblastic leukemia (ALL), etc.], multiple myeloma, Non-Hodgkin lymphoma, Chronic lymphocytic leukemia (CLL), Hodgkin lymphoma, T-cell lymphoma, bone marrow failure syndromes, myelodysplastic syndrome (MDS), clonal hematopoiesis of indeterminate potential (CHIP), clonal cytopenias of undetermined significance (CCUS), aplastic anemia, and metastatic solid tumors that travel to the bone marrow (lung, breast, kidney, prostate, thyroid, etc.). It is understood that hemopoietic stem cells found in this domain may be quiescent or proliferating. It is understood that hemopoietic stem cells found in this domain may be quiescent, proliferating or differentiating.

Diseased state cells can include, but are not limited to, macrocytes, polychromataphilic reticulocytes, aggregate reticulocytes, punctate reticulocytes, target cells, spherocytes, ovalocytes/elliptocytes, stromatocytes, sickle cells, acanthocytes, schistocytes, helmet cells, dacrocytes/teardrop cells, echinocytes/Burr cells, Pappenheimer bodies, Cabot ring cells, punctate basophilia/basophilic stippling cells, Heinz-Endrich bodies, codocytes/leptocytes, megaloblastic cells, hypochromic red blood cells, microcytic red blood cells, macrocytic red blood cells, knizocytes, degmacytes, fragmented red blood cells, Thalassemia red blood cells, Bite cell red blood cells, Hemoglobin C Crystal red blood cells.

The diseased state cells can also include cells of bone marrow cancers, including mature cancer cells and those undergoing angiogenesis, including, but not limited to, multiple myeloma cells and multiple myeloma precursor cells (cells exhibiting monoclonal gammopathy of unknown significance and smoldering myeloma cells), leukemic stem cells, leukemic blast cells, and leukemic promyelocytes.

The modifier “about” used in connection with a quantity is inclusive of the stated value and has the meaning dictated by the context (e.g., includes the degree of error associated with measurement of the particular quantity). In some embodiments, the term “about” indicates the stated value plus or minus 10%. In other embodiments, the term indicates the stated value plus or minus 5%. In other embodiments, the term indicates the stated value, plus or minus 2%.

Much of the remaining detailed description is specific to bone-related embodiments. However, it should be understood that the following disclosure is also applicable to non-bone-related embodiments.

Bone tissue is a heavily calcified organic-inorganic nanocomposite that is densely populated with active cells. Despite significant progress on engineering of complex tissues in the lab, strategies that replicate such fundamental characteristics of bone tissue in-vitro have remained non-existent. Accordingly, there currently are no strategies where cells are embedded in 3D matrix materials that undergo directed mineralization to mimic the native bone nanoscale structure, composition and function. Here, a biomimetic approach is described to guide the deposition of nanoscale apatite in the intra- and extrafibrillar spaces of collagen encapsulated with osteoprogenitor, vascular and neural cells. This process replicates the key hallmarks of the bone cellular and extracellular microenvironment, including its protein-guided process of biomineralization, nanostructure, vasculature, and ability to stimulate osteogenic differentiation in the absence of osteoinductive supplements. Ultimately this approach allows for on-demand fabrication of nanoscale-mineralized and vascularized bone-like tissues in-vitro with unprecedented levels of biomimicry.

The native bone extracellular matrix consists of an intricate structure that is constituted primarily of type I collagen fibrils co-assembled with non-collagenous proteins, strengthened by the confined deposition of apatite crystallites.¹ On the ultrastructural level these crystals are arranged in the form of nanosized platelets that are hierarchically distributed both within (intrafibrillar mineral) and between (extrafibrillar mineral) collagen fibrils in the tissue matrix.²⁻⁵ Given the outstanding load bearing function of bone, such an intricate hierarchical distribution of mineral has drawn significant attention in the materials engineering community, and has been shown to be a key determinant to the long-range structure and function relationships of native bone^{6,7}. In bone biomineralization, the deposition of apatite crystals inside collagen fibrils is synergistically orchestrated by matrix non-collagenous proteins^{8,9}, the periodic arrangement of the tropocollagen molecules^{2,10}, fibril geometryl¹¹, and water^{12,13}. Accordingly, non-collagenous proteins may sequester mineral ions to form metastable, liquid-phase nanodroplets of amorphous calcium phosphate^{14,15}, which penetrate the interstices of collagen fibrils via capillary and electrostatic interactions¹⁶⁻¹⁸, later transforming into thermodynamically stable carbonated, calcium deficient hydroxyapatite¹⁹. Recent efforts have been able to mimic such a process in vitro with increasing levels of success^{10,16,18,20-23}. These have included the use of poly(amino acids) and synthetic organic polyelectrolytes early on²⁴, and have recently explored the use of self-assembling peptide-amphiphiles^{21,22} and anionic polymer acids to mimic the function of non-collagenous proteins in templating hydroxyapatite growth within collagen fibrils in vitro^{10,14,16,18,20,25}. Nevertheless, these strategies have been unable to mimic the cell-rich characteristic of bone tissue, and hence have found limited use as model systems to study bone function, disease progression, or response to drugs and repair.

Cell-based approaches to mimic human bone in the lab have relied heavily on the use of pre-calcified materials, such as brittle ceramics^{26,27} or simulated body fluid-treated scaffolds that are post-seeded with osteoprogenitor cells²⁸. Although relevant to bone regeneration²⁹, these systems utilize cells in two-dimensional monolayers seeded within relatively large pores and thus oversimplify the complexity of the 3D bone microenvironment. Moreover, they are unable to accurately reproduce the gradual entrapment of osteoprogenitors in the bone matrix in the form of osteocytes, which represent over 90% of bone cells and regulate bone function in a paracrine manner, from inside-out³⁰. Cell-laden polymeric hydrogels^{31,32}, which have been proposed as an alternative, may more closely approximate the 3D nature of the cell-laden bone matrix. However, they too fail to replicate the complexity of bone's nanoscale calcification and mineral formation is typically restricted to small and dispersed nodules that appear after 14-21 days of culture in vitro³³. Model systems that controllably replicate the heavily calcified bone extracellular microenvironment with nanoscale precision, while being densely populated with multitypic human cells should allow for extensive experimental manipulation, tunability and throughput, while also enabling unprecedented analyses of cell response to essential cell-matrix and cell-mineral interactions naturally occurring in bone.

Disclosed herein is the encapsulation of undifferentiated human mesenchymal stem cells (hMSCs) in 3D microenvironments, cultured in supersaturated calcium and phosphate-rich cell media supplemented with a non-collagenous protein analog, which directs the formation of nanoscale hydroxyapatite in the interstices of collagen fibrils. This process mimic the nanoscale structure, mineral composition, and a set of important biological function that are characteristic to the of the cell-rich calcified bone microenvironment. The matrix nanoscale mineralization disclosed herein creates a bone-like microenvironment that alone stimulates osteogenic differentiation of stem cells without the requirement for osteoinductive supplements. Moreover, this process lead to cell morphology and cell-matrix interactions that were consistent with the characteristics of pre-osteocytes embedded in mineralized bone. The methods and compositions disclosed herein enable the formation of pericyte-supported blood capillaries and integrated neuronal networks that are cemented within a bed of dense minerals, both of which may address the long-standing challenges of engineering vascularized and innervated bone-like tissues in vitro. For example, this model system engineered tissue may stimulate homing of engrafted prostate cancer cells in vivo Additionally, we show that hMSCs embedded together with endothelial cells in such mineralized microenvironments form robust pericyte-supported blood capillaries in-vitro and in-vitro, and stimulate the engraftment and growth of prostate cancer adjacent to bone-like tissue constructs in-vivo; thus mimicking the known stimulatory effect of bone tissue on prostate cancer cells. In summary, the methods and compositions disclosed herein may allow for controlled engineering of nanoscale mineralized, vascularized, bone-like model systems with high levels of nanoscale biomimicry and desirable biological functions, which may have broad applications for drug discovery, regenerative medicine, and various aspects of bone research.

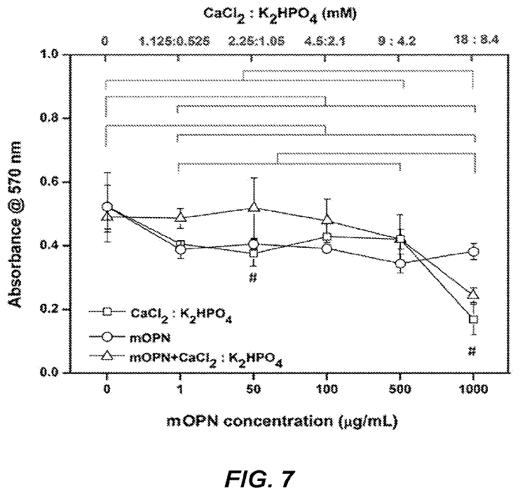

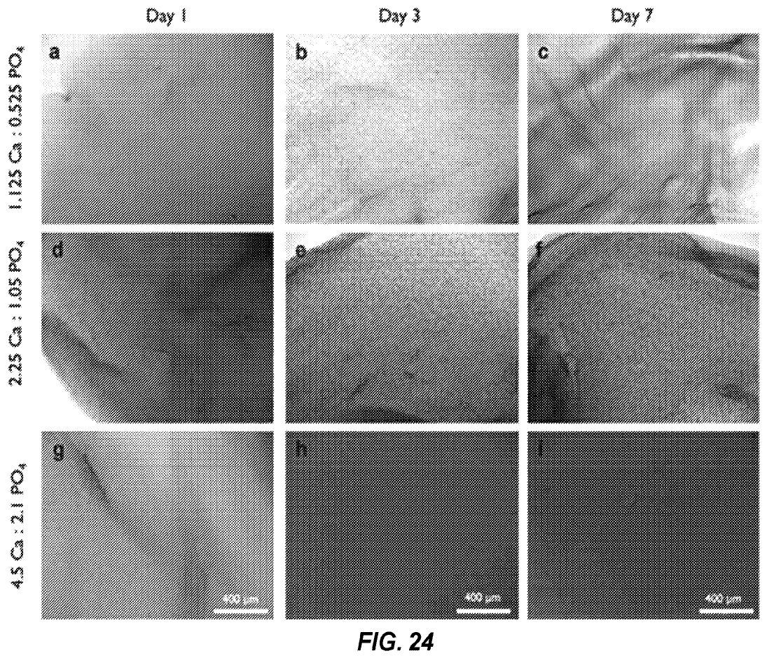

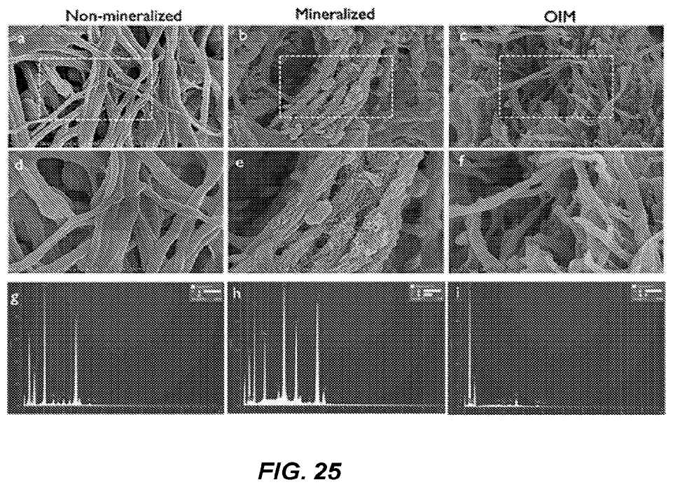

In native bone tissue, the extracellular levels of Ca and P ions are supersaturated with respect to hydroxyapatite, so their precipitation is tightly controlled by anionic matrix proteins,⁶⁰ which purportedly act as nucleation inhibitors. Here, a protein-induced biomimetic mineralization process which uses milk-extracted osteopontin (mOPN) as a anionic protein analogue was adopted to prevent spontaneous precipitation of calcium and phosphate and modulate the non-classical (i.e., amorphous precursor) mineralization process³⁴ within the collagen fibrils throughout the cell-laden matrix in a rapid fashion. Media containing varying molar concentrations of Ca²⁺ (1.125-18 mM) and PO₄³⁻ (0.525-8.4 mM) in combination with different concentrations of mOPN (1-1000 μg/mL) were first screened to test for cell compatibility (FIG. 7). High concentrations of Ca²⁺ and PO₄³⁻ had a significant cytotoxic effect, which can be attributed to the osmotic imbalance between the cell cytoplasm and the medium. When ions were complexed with mOPN, however, such cytotoxic effects were significantly reduced (FIG. 7). In addition, mOPN treatment alone did not reduce the viability or metabolic activity of hMSCs, regardless of the dosage used. The rate of mineral formation in collagen hydrogels was determined by comparing values within a lower range of Ca²⁺ and PO₄³⁻ concentrations that were not cytotoxic (FIG. 24). Within such a range, cell medium supplemented with 4.5 mM Ca²⁺ and 2.1 mM PO₄³⁻, stabilized with 100 μg/mL mOPN, which are in the higher range of levels of Ca²⁺ and PO₄³⁻ typically reported for extracellular fluids in the body³⁵, was chosen for the remainder of the experiments due to the greater efficiency of matrix mineralization and lack of cytotoxicity toward hMSCs. Next, cell-laden mineralized tissue constructs were generated by encapsulating hMSCs in a fibrillar collagen hydrogel (1.5 mg/mL) and the gels were exposed to the supersaturated calcium and phosphate solution stabilized with mOPN at the aforementioned concentrations for three days, so as to induce both intra- and extrafibrillar nanoscale mineralization in the matrix surrounding the cells.

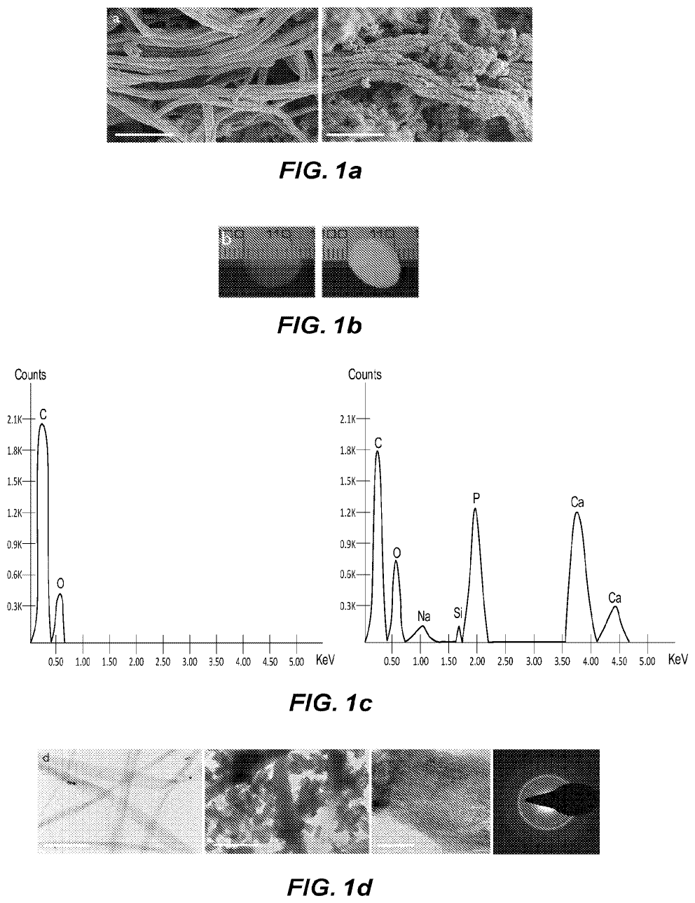

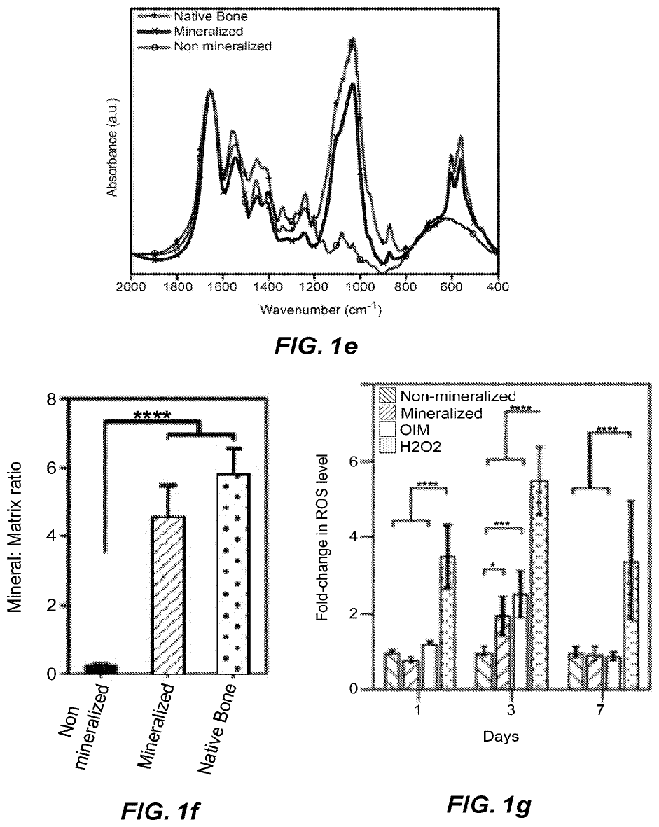

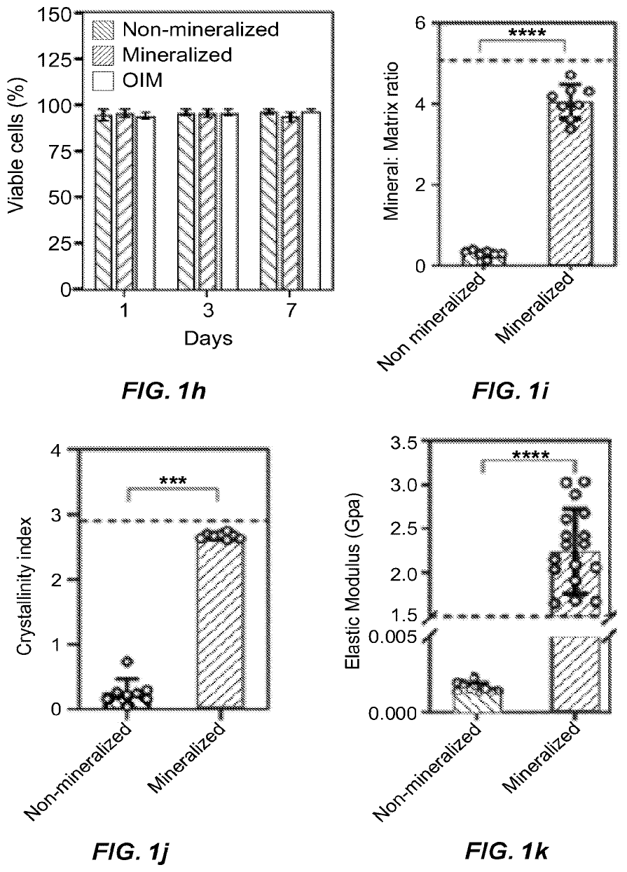

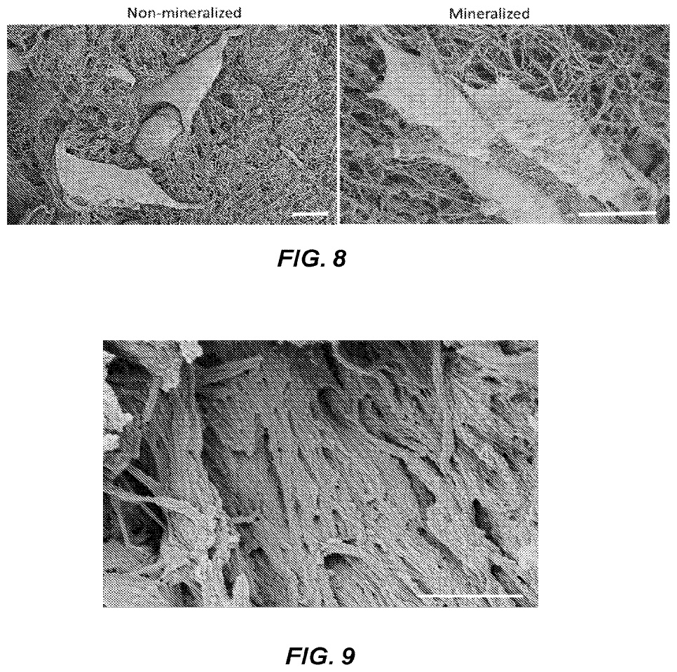

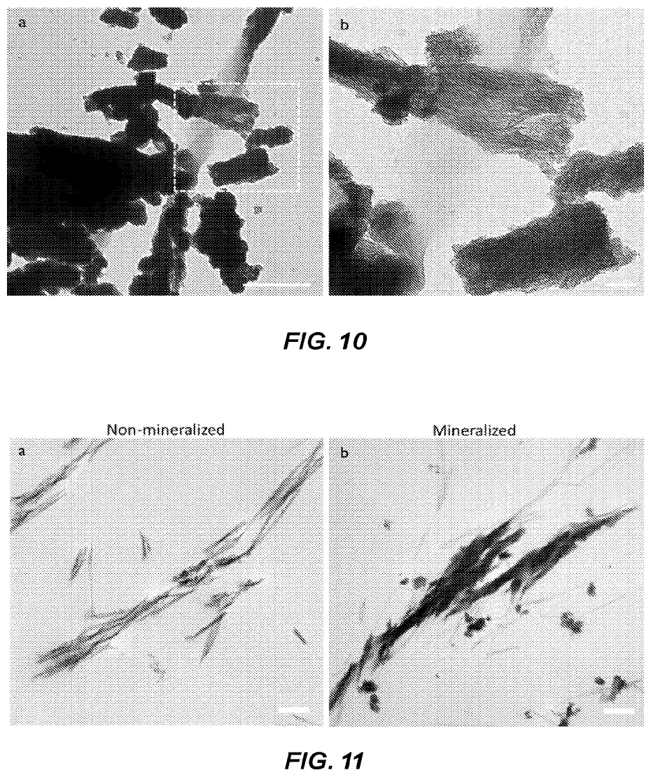

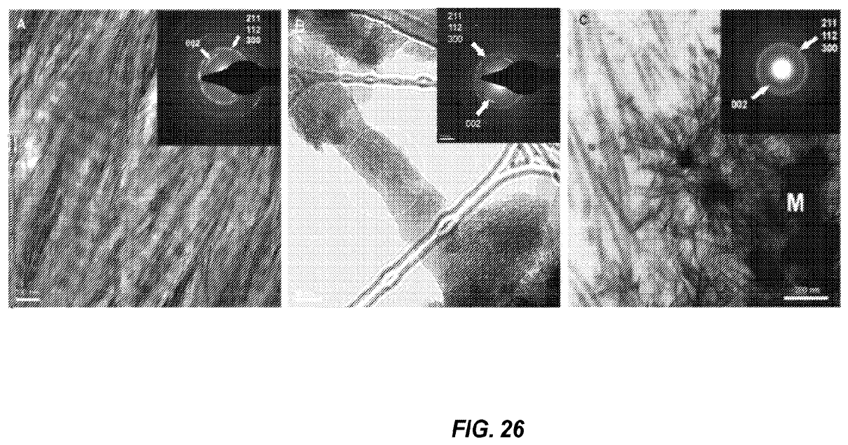

Then the nanostructural properties and mineral composition of the treated cell-laden hydrogels after the 3-day mineralization process were examined. SEM analyses of mineralized samples showed a visible distinction from non-mineralized controls, pointing to the presence of homogeneously distributed extrafibrillar calcium and phosphate deposits (FIG. 1a and FIG. 8). The gross appearance of the hydrogels changed from transparent to an opaque white after 3 days of culture under the controlled mineralization conditions (FIG. 1b). Energy-dispersive spectroscopy (EDX) profiling of mineralized samples revealed Ca and P as the major constituents of the matrix, with a Ca/P ratio of approximately 1.6, a value that approximates that of native bone apatite (FIG. 1c). Furthermore, transmission electron microscopy (TEM) imaging of mineralized samples confirmed the preferential intrafibrillar localization of apatite crystallites, and the apparent presence of nanosized platelets oriented parallel to the long axis of the fibrils (FIG. 1d and FIG. 10) hiding the typical banding pattern of non-mineralized collagen type 1 (FIG. 1d). Selective area electron diffraction (SAED) analyses of mineralized fibrils revealed the typical broad arcs for the (002) plane and overlapping arcs for the (112), (211), and (300) planes, all of which were consistent with the known hexagonal crystal form of hydroxyapatite in the native bone and in osteoblast-secreted apatite crystals (FIGS. 1d and 26) 3,36. In native bone, a significant proportion of mineral apatite resides on the extrafibrillar space, which is estimated to be as high as 70% of the total mineral volume in the tissue. 19,61 Therefore, 3D tomographic images of mineralized collagen fibrils whereby collecting a series of images as the TEM stage was tilted from +70 to −70° (FIG. 11). The ultrastructural features of the mineralized constructs revealed mineral accumulation in both intra- and extrafibrillar compartments, with a substantial amount of the mineral being situated in the extrafibrillar space (FIGS. 1d, 26, 10, 13), akin to native bone (FIG. 9). It should be noted that this type of extrafibrillar mineral still seems to be comprised of nanoscopic crystals, unlike the large spherulitic hydroxyapatite plates that form without the protein additive.

The chemical composition of our mineralized samples in comparison to that of native bone was further characterized by using Fourier Transform infrared spectroscopy (FTIR) (FIG. 1e). The peak intensities and positions of apatitic phosphate (1030, 600, and 560 cm⁻¹), carbonate (874 cm⁻¹) and amide I, II, and III (1649 cm⁻¹, 1553 cm⁻¹, 1248 cm⁻¹) bands for the mineralized matrix were found to be similar to that of bone tissue. Similarly, although the absolute levels of mineral and collagen were inferior to those in native bone, given the low density of the collagen hydrogels in comparison to that of the native tissue, the relative mineral-to-matrix ratio (FIG. 1f) and crystallinity index (FIG. 1j) of the mineralized scaffolds approximated that of native bone.

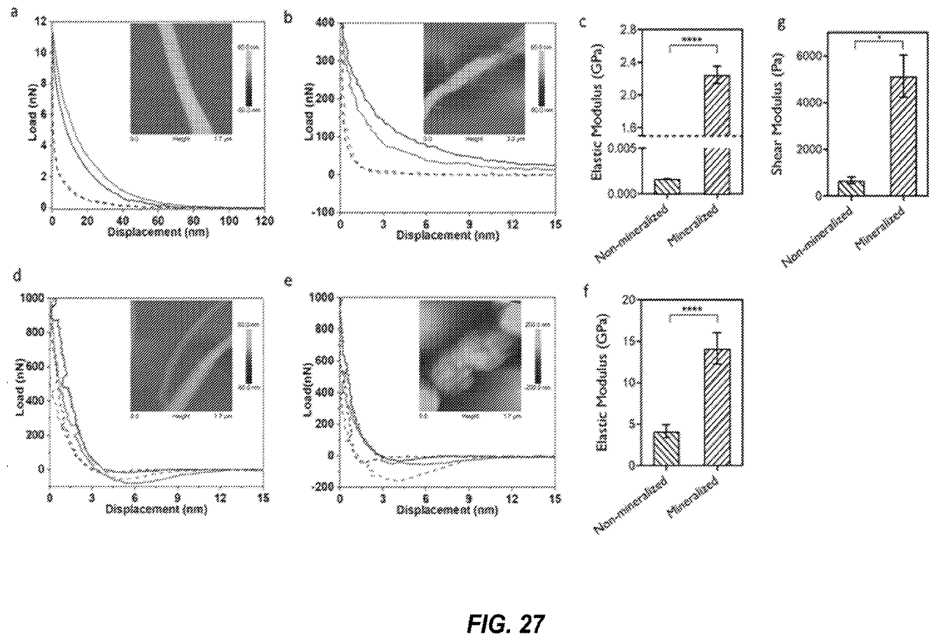

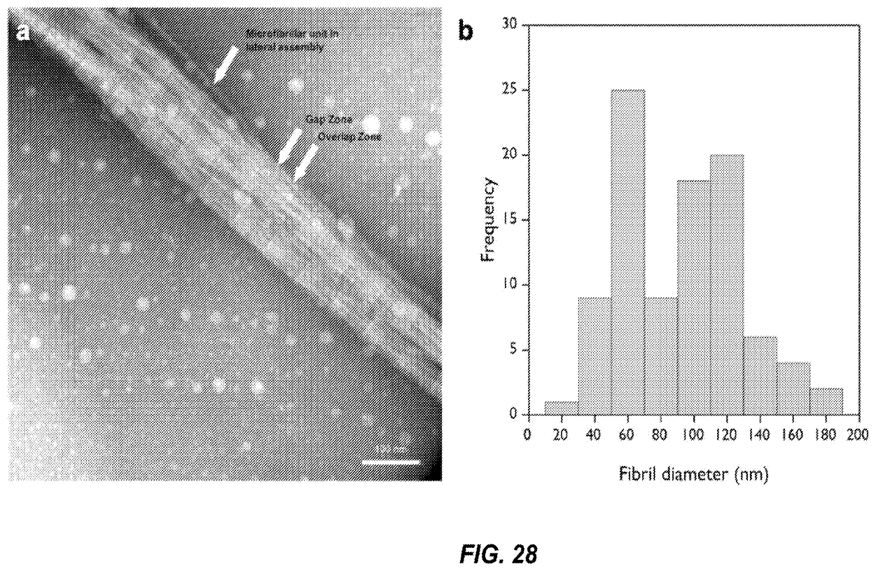

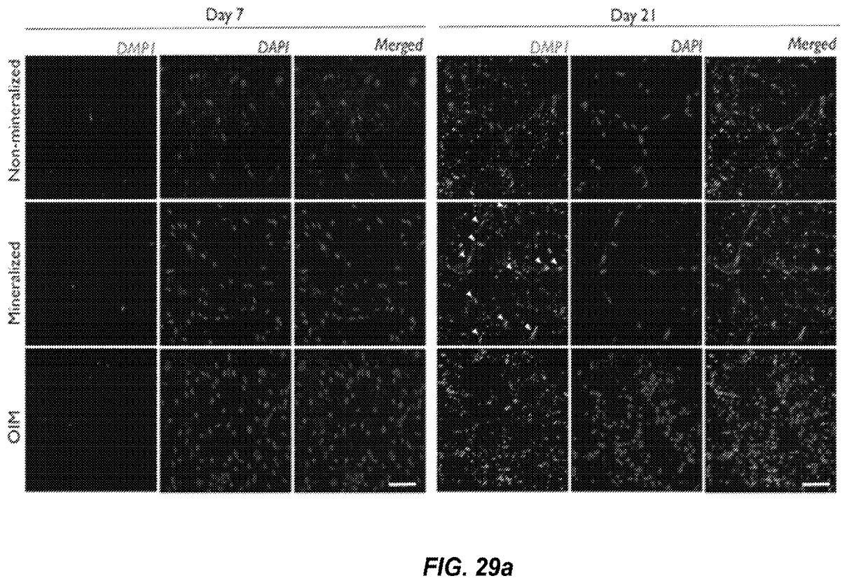

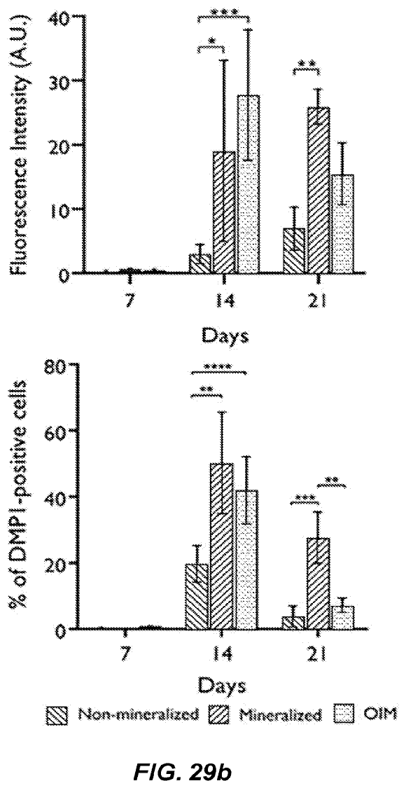

To determine the ability of the apatite mineral to bind to and mechanically reinforce the fibrils, AFM nanoindentation was performed on individual collagen fibrils in solution and ambient air (FIG. 27), either before or after mineralization. The hydrated non-mineralized collagen had an elastic modulus of 0.0016±0.0003 GPa, while the elastic modulus of mineralized fibrils was 2.21±0.046 GPa (FIG. 1k). Despite the significant increase in nanoindentation modulus of individual fibrils, the overall stiffness of the hydrogel constructs was still markedly lower than that of bone, which is in the order of 20 GPa on a macroscale (FIG. 27). Moreover, the fibrillar organization, diameter and D-banding periodicity of the collagen matrix were also reminiscent of that in native bone (FIG. 28).

Overall, both the mineral composition and nanostructural organization closely mimic that of bone tissue.

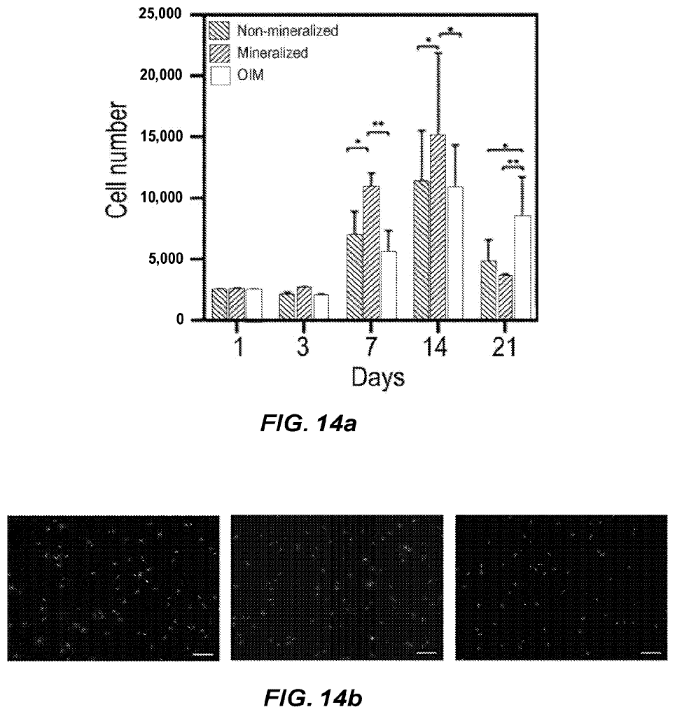

Evaluated next was whether the nanoscale mineralization of cell-laden hydrogels could lead to reduced cell viability due to increased osmotic damage or physical impairment of nutrient delivery to the cells in the matrix. Approximately 90% of cells embedded in the mineralized hydrogels remained viable after at least 7 days of culture in vitro, which was similar to both non-mineralized collagen and collagen treated with osteoinductive medium (OIM) as controls (FIGS. 14b and 1h). Then, whether the mineralization process could influence the generation of intracellular stress and the proliferation of cells in the cell-laden tissue constructs was evaluated. During the 3 days of exposure to the guided mineralization process, there was no significant increase in the levels of reactive oxygen species (ROS) on day 1 and a comparable increase found on day 3 where both mineralized and OIM-treated samples generated more ROS than non-mineralized controls. After 7 days, both treatments had comparable levels of ROS as non-mineralized controls and all groups were significantly lower than H2O2, which was used as a positive control for stress generation on the cells (FIG. 1g). The proliferation of encapsulated hMSCs in response to the gradual matrix mineralization was then examined. Cell proliferation between non-mineralized, mineralized, and OIM-treated groups were comparable from day 1 to day 3, and after 7 and 14 days cell proliferation was still present but significantly higher in mineralized samples (FIG. 14a). On day 21, proliferation decreased in all three groups, potentially due to lineage commitment of cells to an osteogenic phenotype or growth impairment due to cell-cell contact. These findings confirm that the cell-laden hydrogel mineralization process is non-cytotoxic and that neither cell metabolism, nor viability are significantly impaired, despite the dense calcification of the matrix surrounding the cells. This is in stark contrast to the same mineralization medium that did not contain the polymeric process-directing agent.

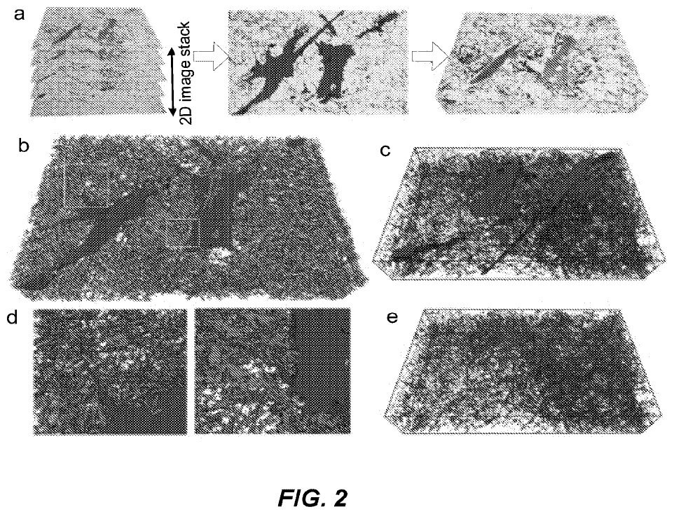

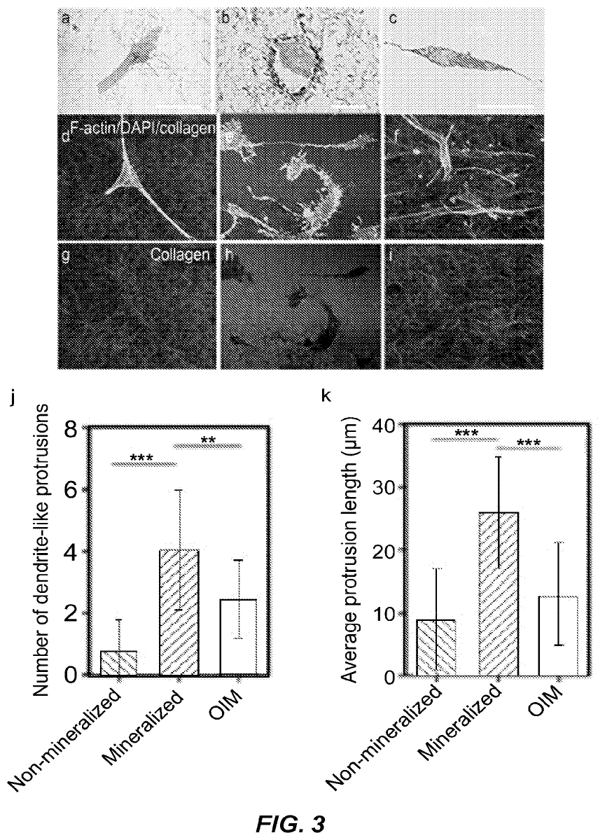

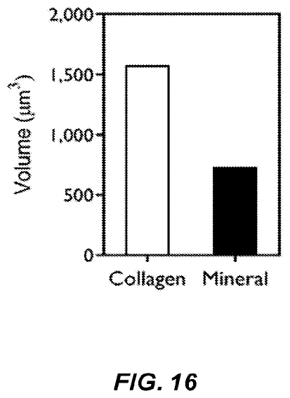

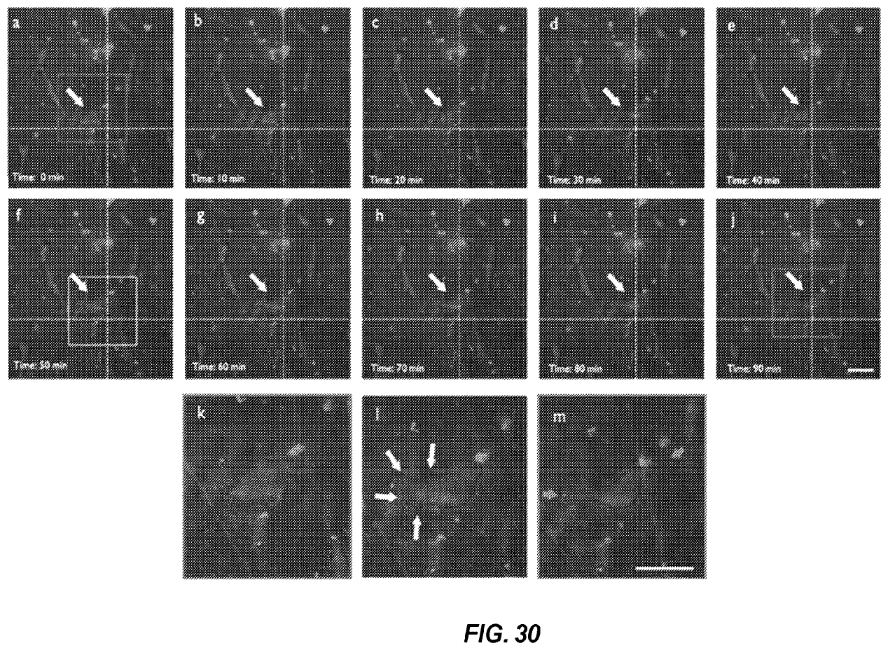

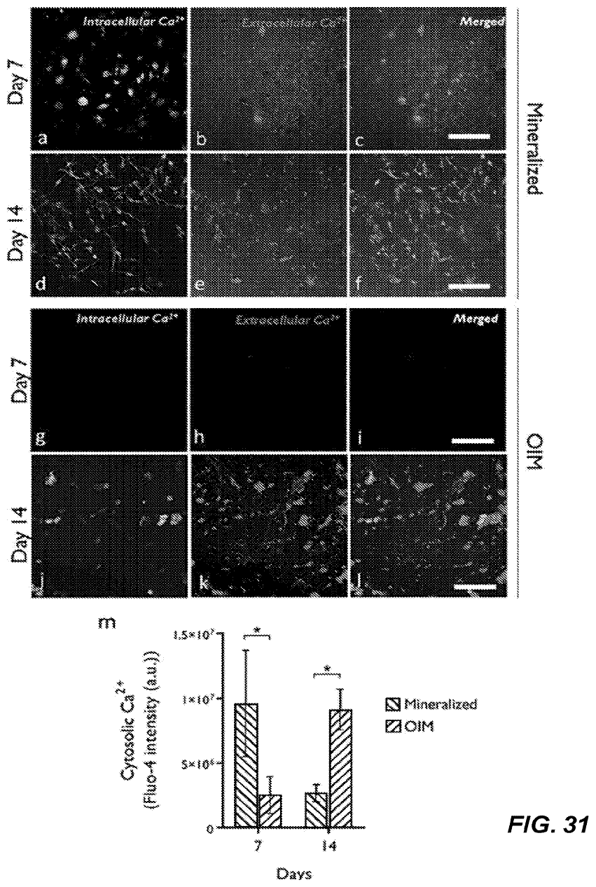

Cells in a 3D microenvironment respond strongly to the structure and mechanics of the matrix in which they are embedded³⁷. Matrix stiffness, especially, is linked to important mechanisms of mechanotransduction-mediated cell differentiation³⁷. Therefore, the structural crosstalk between the encapsulated cells and the matrix before and after the mineralization process was studied. To assess such interactions, a combination of serial-section backscatter SEM imaging and 3D digital reconstruction was used to simultaneously elucidate the ultrastructure of mineralized fibrils along with the microscale architecture of the embedded cells. As a first step, a series of images of mineralized samples at Z-intervals of 60 nm, with the intent of recreating a 3D digital image of the mineralized samples as a function of the contrast generated by the backscatter electrons. A set of 190 slices was used to segment the cells, the mineral-free collagen, and the mineralized fibrils, based upon their respective electron-density contrast difference (FIG. 2 panel a). When viewed in 3D, cells are seen with a well-spread morphology, lying within a bed of densely packed mineralized fibrils (FIG. 2 panel b). Of note, these fibrils are mineralized with similar levels of crystallinity as those observed in native bone and in osteoblast-secreted minerals (FIGS. 26, 10, 13). Cells interacted closely with the mineral and extended dendrite-like projections that are characteristic of an osteocyte-like phenotype (FIG. 2 panels c, d). These long cell processes are consistent with the ones visualized in actin-stained cells, shown in FIG. 3e. Regions adjacent to the embedded cells appeared more densely compacted with mineral (FIG. 2c). This indicates that even though ˜50% of the organic matrix was mineralized (FIG. 16), cells were still able to move within the surrounding matrix (FIG. 30), secrete soluble proteins, as well as process intracellular and extracellular calcium (FIG. 31), all of which may be indicative of active new tissue formation.

Further differences in the ultrastructural matrix organization of non-mineralized (FIG. 3 panel a), mineralized (FIG. 3 panel b) groups can be visibly distinguished from the reverse contrast SEM images obtained from resin embedded samples. FIG. 3 panel b depicts a single cell inside a hollow space surrounded by mineralized matrix, which shares strong similarities to the native bone lacunae. The mineral deposits appeared as dark contrasted regions that can be distinguished from the pale non-mineralized collagen. Notably, the contours of these lacuna-like cavities where cells resided, were bordered by a denser layer of mineral, which is consistent with the characteristics of peri-lacunar features surrounding native osteocytes, such as the lamina limitans found in osteonal bone.²⁸ None of the aforementioned ultrastructural features were found in non-mineralized controls (FIG. 3 panel a). Interestingly, in native bone, osteoblasts go through a series of morphological changes as the surrounding matrix transforms from soft osteoid to more heavily mineralized bone³⁰. Consistent with that notion, fluorescence images obtained from cells cultured for at least 7 days in the mineralized microenvironment, showed cells extending narrow actin-rich dendritic-like processes that appeared to radiate through the matrix toward neighboring cells (FIG. 3 panel e). These morphological features resemble the characteristic morphology of osteocytes, where cell processes precede and guide dendrite growth and orientation⁴⁸. In non-mineralized controls (FIG. 3 panel d) or OIM-treated controls (FIG. 3 panel f), on the other hand, cells lacked any marked increase in the projection of cell dendrites (FIG. 3 panels j and k). Furthermore, images of the collagen matrix obtained in confocal reflectance mode revealed uniform collagen fiber organization in the non-mineralized (FIG. 3 panel g) and OIM-treated controls (FIG. 3 panel i), whereas well-defined fluorescent-less features that appeared devoid of collagen and resembled the appearance of bone lacunae were noted in the mineralized constructs (FIG. 3 panel h).

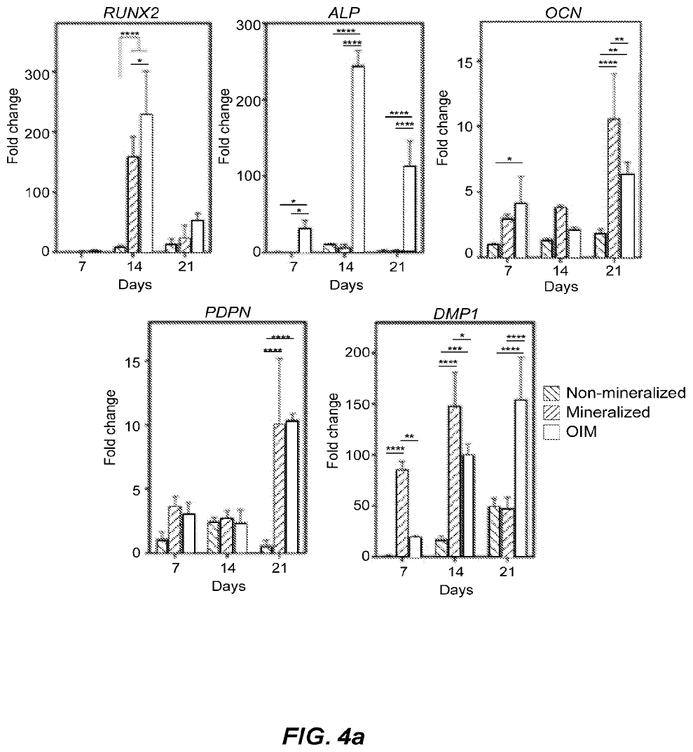

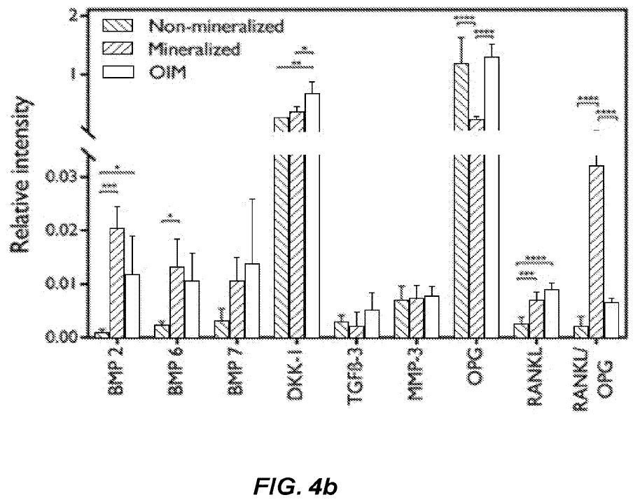

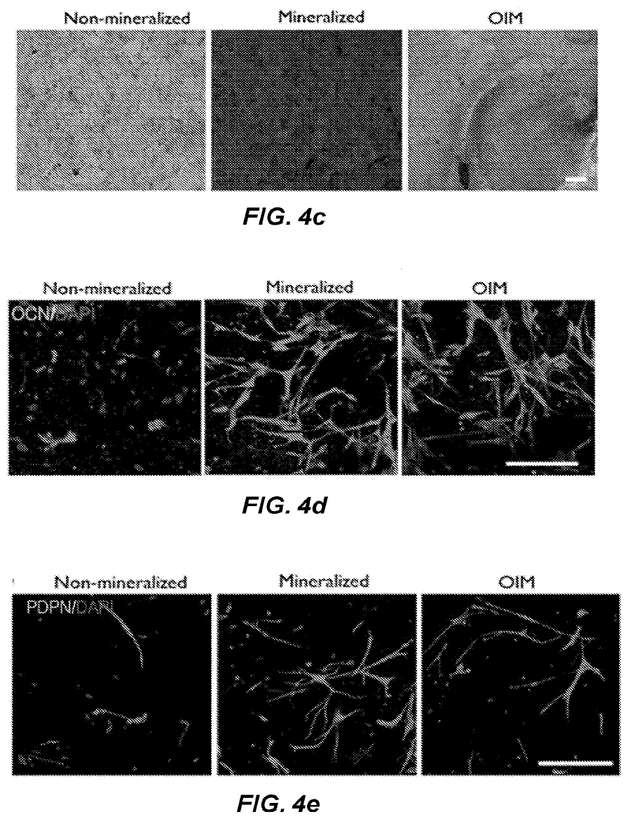

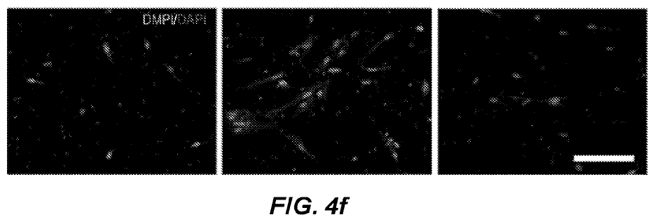

Since the electron and fluorescence microscopy images suggested that, despite not being exposed to any osteoinductive supplements, hMSCs underwent morphological and ultrastructural changes shortly after matrix mineralization, the expression of key osteogenic markers soon after mineralization was completed were assessed. To assess that, the transcript levels of major genes associated with osteoblastic and early osteocytic differentiation of hMSCs were surveyed. These expression levels were compared against cells cultured either in non-mineralized controls, or in collagen cultured in the presence of known osteoinductive agents, such as ascorbic acid, dexamethasone, and B-glycerol phosphate. Cells in the mineralized matrix exhibited either significantly higher or comparable gene expression profiles to those obtained using osteoinductive media (FIG. 4a), with the exception of runt-related transcription factor 2 (RUNX2) and alkaline phosphatase (ALP) at specific time points. There was a significant increase in the expression level of RUNX2 in the mineralized construct after 14 days in comparison to non-mineralized collagen. Similarly, the expression levels of osteocalcin (OCN), a late stage marker for osteoblastic differentiation, had significantly higher expression in mineralized samples than the positive control after 21 days, wherein nearly a 2-fold increase was observed^{39,40}. Likewise, matrix mineralization alone induced an early onset in the expression of DMP1, a marker that has been shown to be predominantly expressed in osteocytes but not in osteoblasts⁴¹. This was nearly 4 times higher than cells cultured in osteoinductive medium after 7 days and still significantly higher on day 14. The expression of PDPN, a mucin-type protein highly expressed in osteocytes, was also comparable between mineralized samples and positive control at all time points. These results support the conjecture that mineralization of the surrounding matrix may be a key determinant for osteoblast-to-osteocyte transition, both in vitro⁴² and in vivo⁴³.

Since both osteoblasts and osteocytes synthesize proteins that participate in bone homeostasis in a paracrine manner^{30,44,45}, the expression of a set of key proteins involved in bone metabolism (BMP-2, -6 and -7, DKK-1 and TGF-β), and remodeling (MMP-3, OPG and RANKL, RANKL/OPG) were analyzed (FIG. 4b). Of the three osteoinductive BMPs tested, the expression of BMP-2 was significantly higher in both the mineralized hydrogels and osteoinductive medium when compared to non-mineralized controls. On the other hand, a significant increase in the secretion of BMP-6 was detected only in the mineralized samples. Conversely, an increased expression of DKK-1, an osteogenic inhibitor, was found in cells treated with osteoinductive medium, compared to the mineralized constructs. With respect to the factors that are involved in bone remodeling, a 5-fold drop in expression of osteoprotegerin (OPG) was noted in the mineralized constructs (FIG. 4b). With respect to the factors that are involved in bone remodeling (MMP-3, OPG, RANKL), a fivefold drop in expression of osteoprotegerin (OPG) was noted in the mineralized constructs compared with both other groups (FIG. 4b). The expression of RANKL, which is one of the crucial cytokines for osteoclastogenesis⁴⁶, was significantly higher in both mineralized and positive control samples when compared to non-mineralized control. Furthermore, given that the balance between RANKL and OPG is a key determinant of bone resorption⁴⁷, we calculated the ratio of RANKL to OPG in our samples, and found a marked increase in mineralized samples, which suggests the ability of cells embedded in the mineralized matrix to secrete signaling factors that orchestrate bone remodeling in a paracrine fashion, akin to native bone.

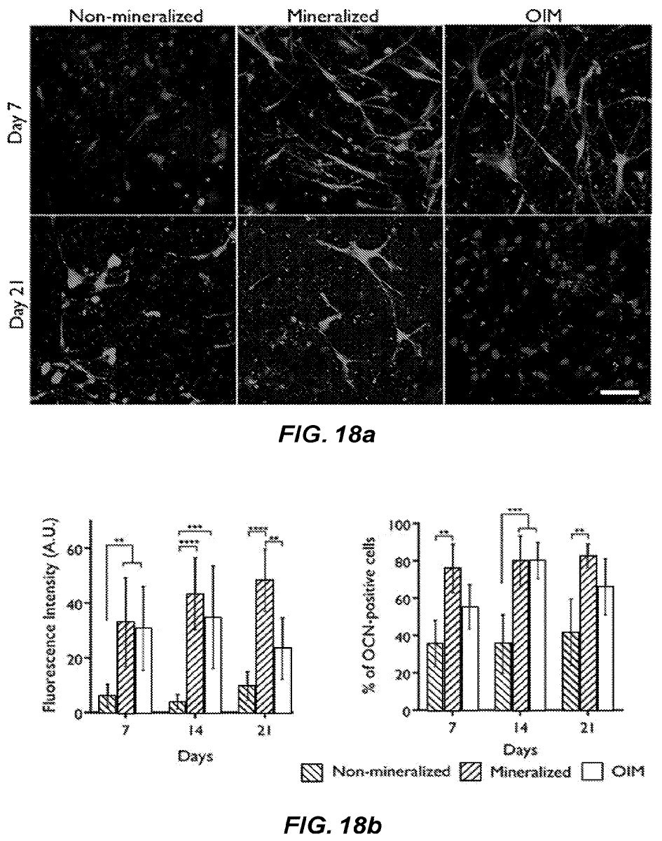

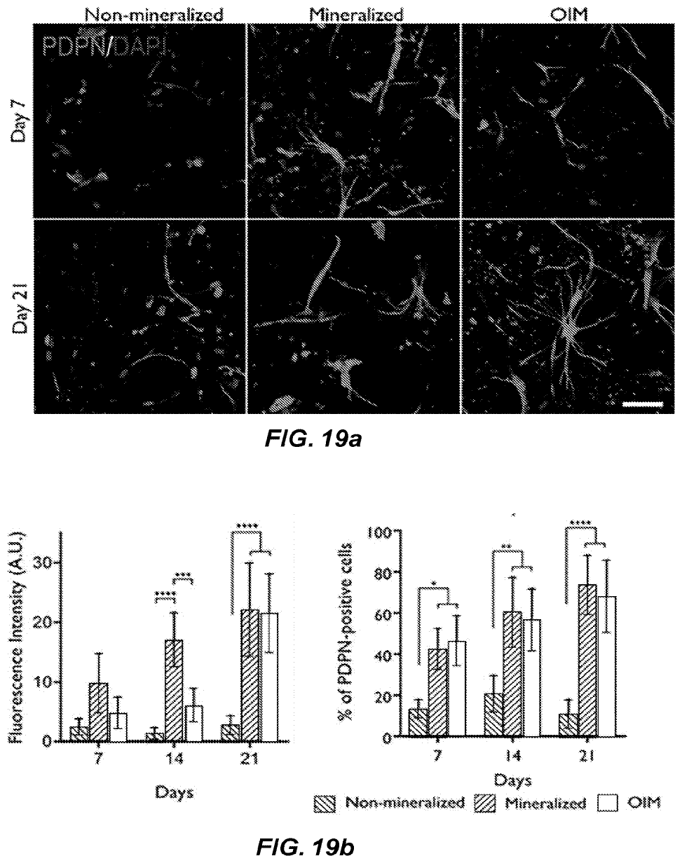

To validate the protein level expression of osteoblastic and osteocytic markers, cells were immunostained for OCN, PDPN, and DMP1 after 7, 14 and 21 days (FIGS. 4d, 4e, 4f, 18a, 18b, 19a, and 19b). Consistent with the gene expression analysis, all three markers were poorly expressed in non-mineralized hydrogels, but showed a marked increase in expression after 7, 14 and 21 days for both mineralized hydrogels and the positive control, although at different levels. These results correlated with the amounts of calcium, as seen by alizarin red staining on day 7, present in the matrix (FIG. 4c). Collectively, the genotypic and proteomic expression patterns suggest that matrix mineralization alone stimulates expression of both osteogenic and pre-osteocytic markers to levels that are at least comparable to gold standard osteoinductive supplements (OIM).

Overall, these results suggest that, when embedded in a microenvironment that replicates the three-dimensionality, composition and nanoscale structure of the mineralized bone niche, hMSCs may express a multitude of morphological characteristics that are consistent with maturing bone cells, all in the absence of osteoinductive factors and driven primarily by matrix mineralization.

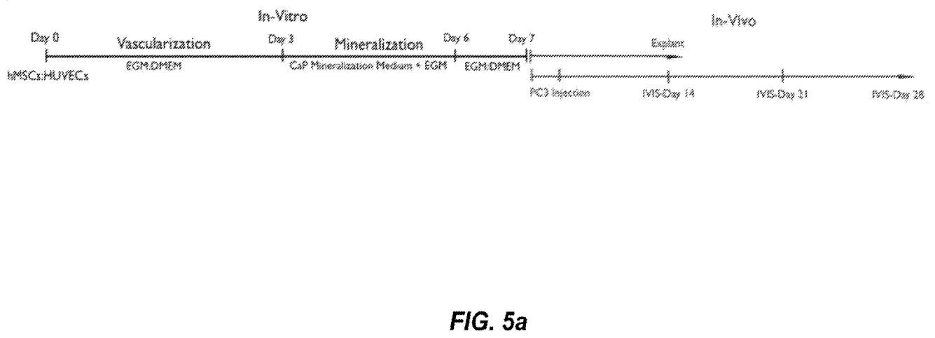

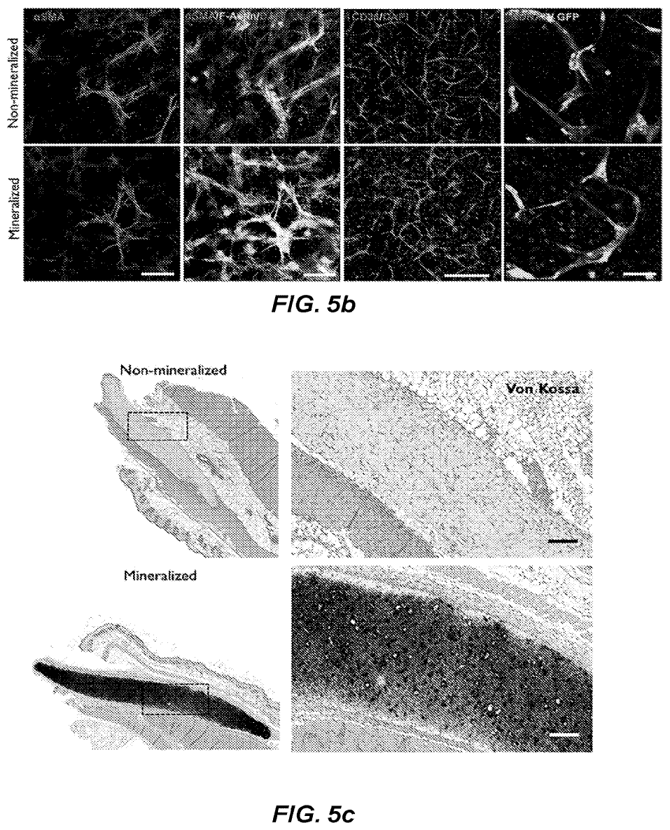

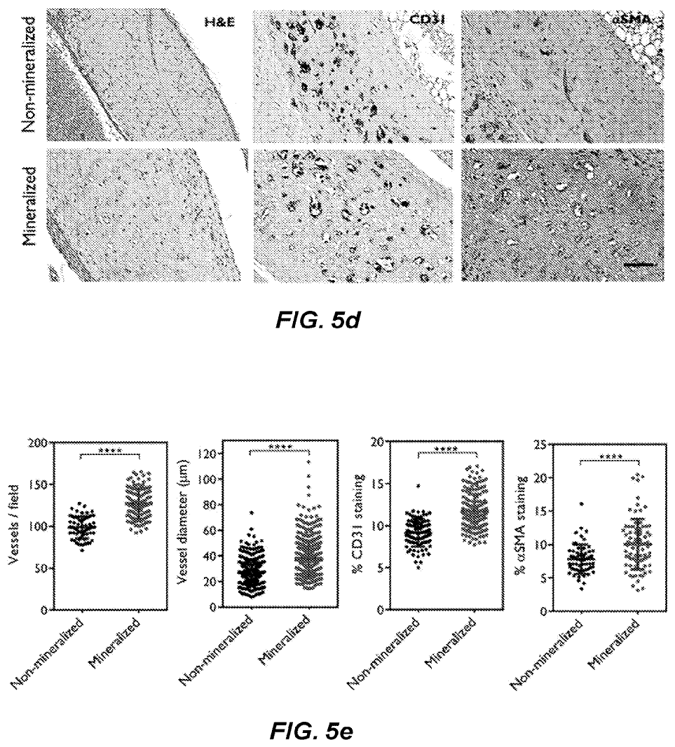

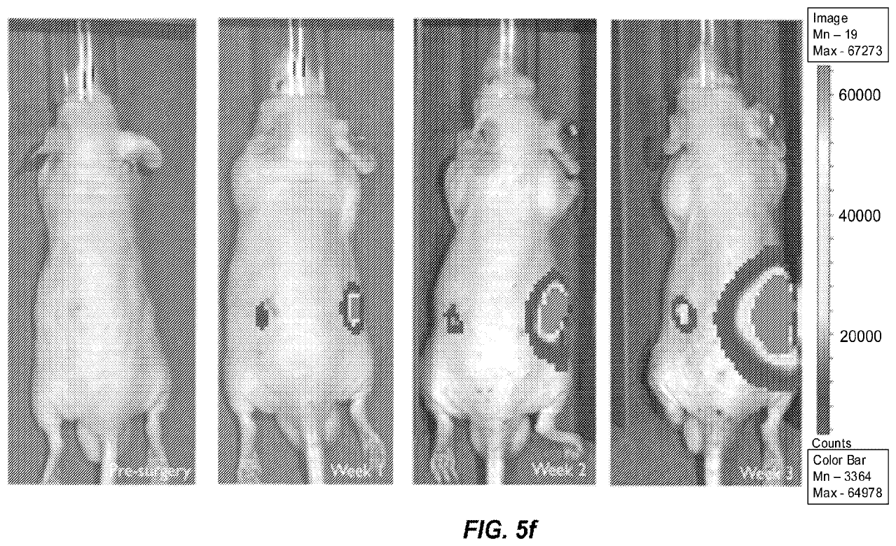

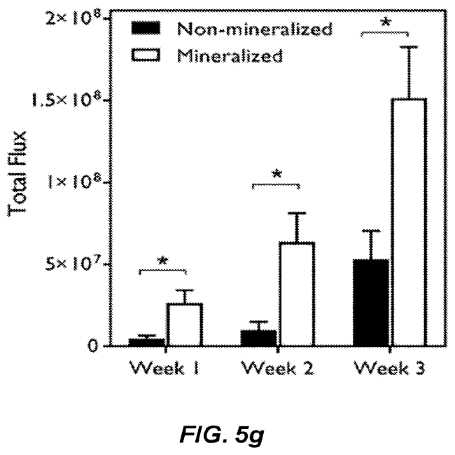

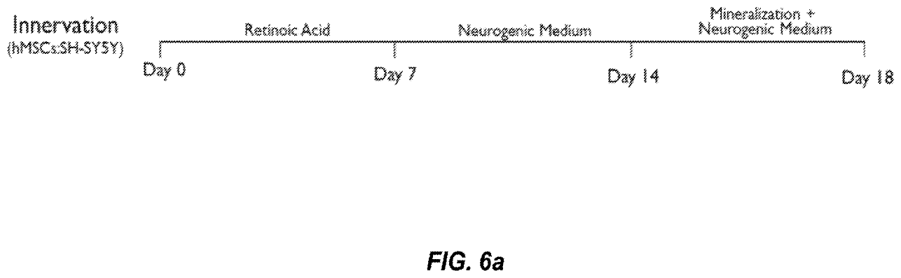

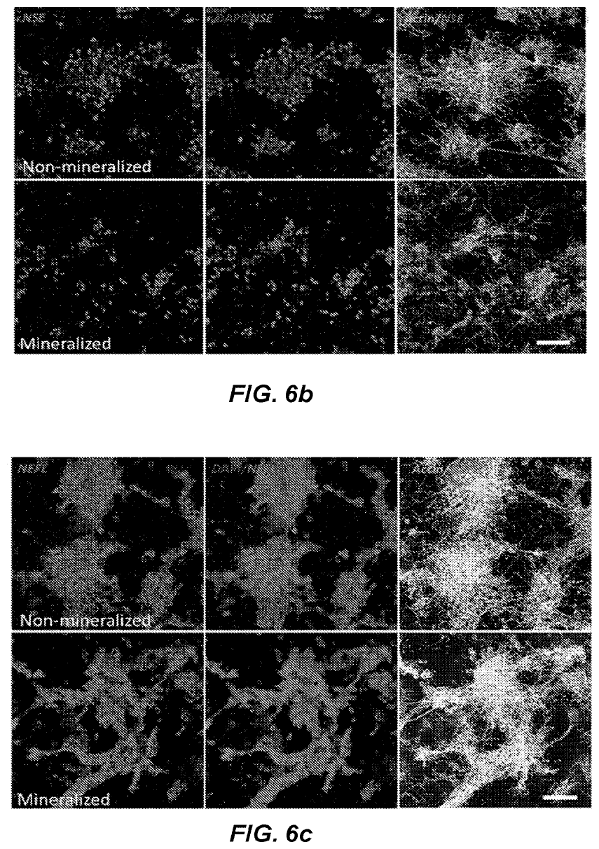

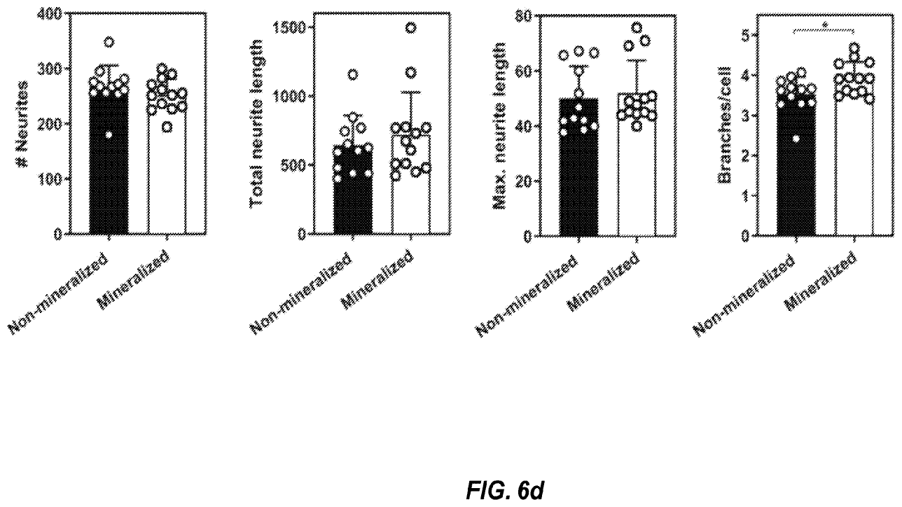

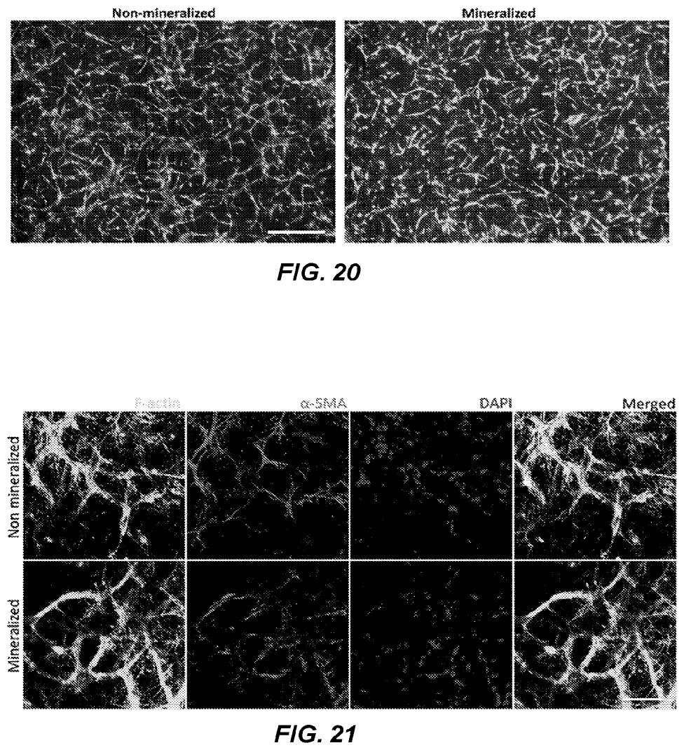

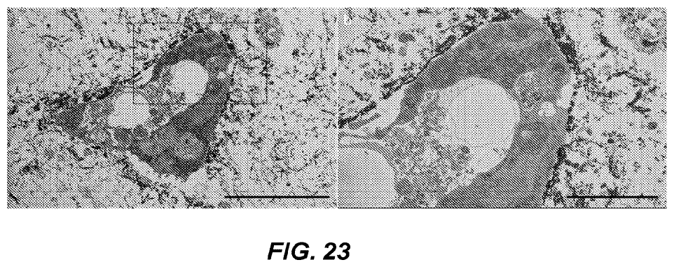

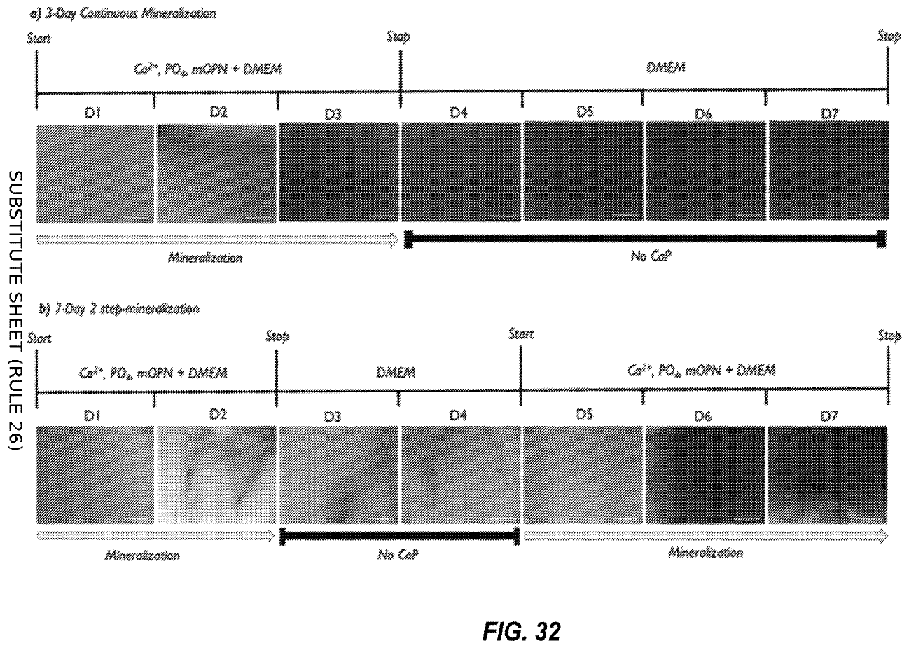

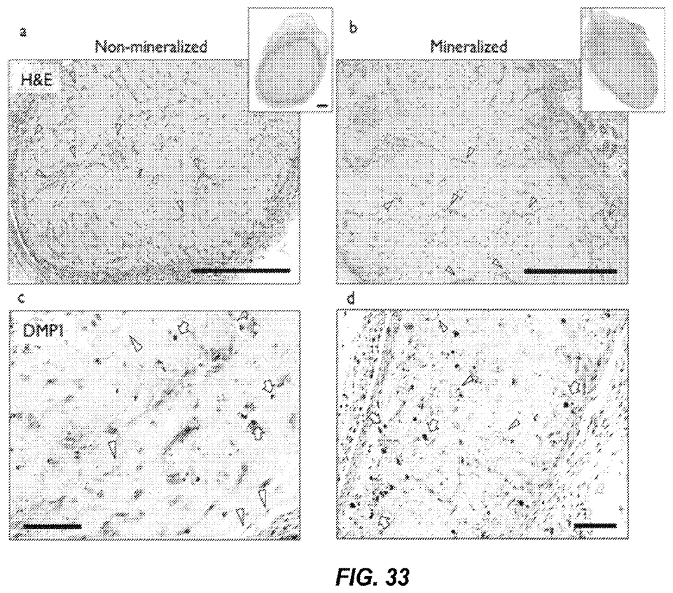

In view of the versatility of the methods and compositions disclosed herein to trigger mineralization in a controlled manner at different time points (FIG. 32), hMSCs were co-cultured with either neuroblastoma (SH-SY5Y) cells or with HUVECs embedded in collagen hydrogels, and allowed neuronal (FIG. 6) and vascular (FIG. 5) networks to form. Then the innervated or vascularized constructs were subjected to the process of nanoscale mineralization (FIGS. 5a, 6a). Of note, during bone formation, vasculature and innervation form prior to the onset of calcification, and the presence of extracellular Ca and P does not impair neither vasculogenesis nor neurogenesis. Thus, whether similar outcomes would be present in the system, with particular emphasis on the formation of vascular networks, which are imperative for tissue grafting in-vivo, were studied. Formation of 18-day old neuronal networks interconnected by actin-rich neurites was indicated by the expression of neurofilament light and neuron specific enolase, which were visible in both mineralized and non-mineralized constructs at comparable levels (FIG. 6b, 6c). Quantification of number of neurites, total neurite length and maximum neurite length were also statistically comparable between mineralized and non-mineralized hydrogels. The number of branches and branch points per cell, on the other hand, were significantly higher for cells embedded in the mineralized constructs. (FIG. 6d). This suggests that the intrafibrillar collagen mineralization process does not hamper the formation of 3D neuronal networks in-vitro. Studies were conducted to determine if vascular networks could be engineered in the core of the collagen scaffolds via endothelial cell morphogenesis, and that such tissues could then be mineralized to form bone-like tissue constructs mimicking the native bone vasculature and nanoscale mineralized matrix. HUVECs and surrounding hMSCs formed vascular tubes within 3 days after cell encapsulation in the collagen hydrogels (FIG. 5b). The introduction of the Ca, P and mOPN supplemented medium induced homogenous mineral deposition throughout the matrix in 3 days (FIG. 23), and HUVECs encapsulated within mineralized hydrogels maintained the interconnected networks (FIGS. 6b, 20-22) and remained strongly positive for the endothelial cell junctional marker, CD31 (FIG. 5b). In addition, hMSCs that co-aligned with the endothelial tubes had a marked expression of a smooth muscle actin (αSMA), which is a marker for differentiation of hMSCs into a pericyte-like phenotype (FIG. 5b, FIG. 21). Of note, differentiation of hMSCs into αSMA-expressing cells in the mineralized constructs was restricted to cells in immediate contact with endothelial tubes, whereas hMSCs that were remote from it maintained their capacity to differentiate into osteoblasts, as indicated by the expression of RUNX2 adjacent to GFP-expressing HUVECs (FIG. 5b). This was consistent with the presence of DMP1+ cells near vascular capillaries in samples collected 7 days post implantation in vivo (FIG. 33).

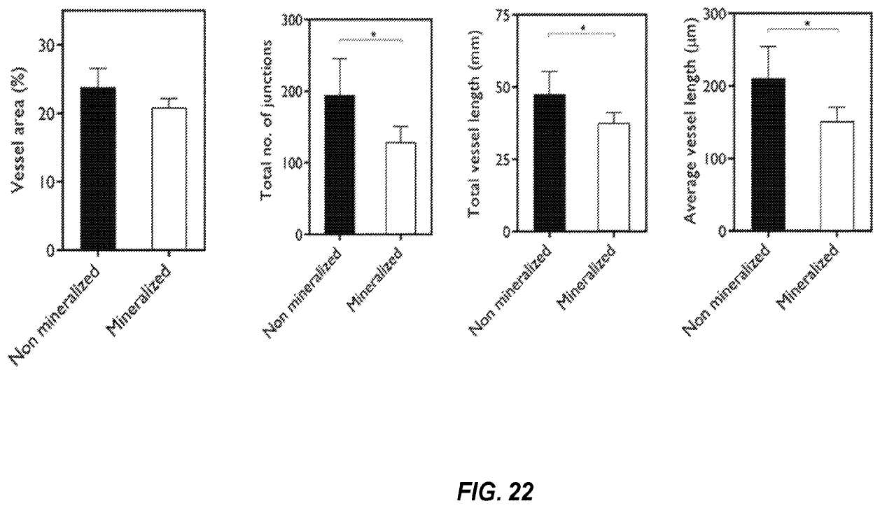

Next, in order to determine the stability of the engineered vascular networks in vivo, the vascularized and mineralized constructs were implanted in the subcutaneous pockets of immunodeficient SCID mice. Histological examination of the mineralized versus non-mineralized constructs harvested after 7 days of implantation showed a high survival of transplanted human cells within both engineered constructs (FIG. 33). Von Kossa staining revealed intense dark calcium deposits homogenously distributed throughout the mineralized constructs, while no mineralization was detected in non-mineralized controls (FIG. 5c). Samples were immunohistochemically stained against human-specific CD31 antibody, and showed tight endothelial cell junctions that are characteristic of functional blood vessels, indicating that those vessels were formed by the implanted human endothelial cells and not by the invading host vessels (FIG. 5d). Interestingly, despite a significant reduction in vessel quantity and size after 7 days in-vitro (FIG. 22), quantification of the microvessel density revealed a significantly higher number of CD31⁺ vessels in the mineralized constructs compared to non-mineralized controls in vivo (FIG. 5e). Also observed was that the mineralized constructs developed much larger sized microvessels than non-mineralized samples. Likewise, the percentage areas of CD31⁺ and αSMA⁺ staining (FIG. 5e) were also significantly higher in mineralized constructs when compared with the non-mineralized controls. Overall, these observations suggest that the endothelial networks were better able to survive in vivo when embedded in mineralized constructs than when formed in non-mineralized collagen.

Lastly, to demonstrate the efficacy of the engineered construct as a model system to study prostate cancer invasion to bone, the mineralized 3D constructs were transplanted into the subcutaneous pockets of immunocompromised mice to create ectopic bone-like microenvironment in vivo. Paracrine signals exerted by the cells in the native bone, as well as matrix molecules released by the actively remodeling mineralized tissue, act as crucial players in providing a conducive environment for the proliferation of disseminated prostate cancer cells. To replicate this phenomenon, 24 hrs post implantation of the cell-laden tissue constructs, a suspension of luciferase expressing PC3 cells was injected directly at the ectopic site, as to determine the ability of our mineralized bone model in conditioning the growth and colonization of tumor cells, similar to the native bone milieu. In vivo bioluminescence imaging was performed over a period of 3 weeks to track the growth of PC3 cells at the target site between mineralized and non-mineralized samples. The incidence of tumor spreading was significantly higher in mineralized samples than in non-mineralized controls at all time points (p<0.05) (FIGS. 5f and 5g). Of note, the target site implanted with the mineralized construct developed almost 3-fold higher bioluminescent signals than the non-mineralized counterparts at the end of 3 weeks. Overall, the results implied that the vascularized and mineralized bone-like tissue constructs could model secondary tumor colonization of prostate cancer (PC3) cells into bone, which may allow for an efficient tool to screen the efficacy of novel therapeutic interventions in cancer to bone metastasis.

Significant efforts have been expended towards engineering bone-like tissue constructs in recent years. Despite substantial progress, engineering of model systems that mimic the key cellular and extracellular characteristics of human bone have remained virtually non-existent thus far. Accordingly, there have been no strategies that enable culture of osteoprogenitor and vascular cells (or other cell types) embedded in a matrix that is controllably calcified on the nanoscale, which are the most fundamental characteristics of human bone. Disclosed herein is that a cell-laden collagen hydrogels can be mineralized to mimic the intra- and extrafibrillar nanoscale mineralization profile of native bone, and that such a microenvironment alone is sufficient to stimulate the osteogenic differentiation of hMSCs, while also enabling the formation of hMSC-supported vascular capillaries in-vitro and in-vivo. Different from traditional osteogenic cell culture protocols, where cells begin to secrete small and dispersed mineral nodules after 14 to 21 days of culture,²⁹ these results show that the approach disclosed herein enables widespread and nearly homogenous (FIGS. 2, 3, and 4c) mineralization of hMSC-laden collagen hydrogels having ultrastructural organization (FIGS. 1a and 1d), elemental composition (FIG. 1c), crystallinity (FIG. 1d) and mineral-to-matrix ratio (FIG. 1i) that are comparable to that of human bone in as little as 72 h. Further, the nanomechanical assessment of individual fibrils (FIG. 1k) were consistent with previous reports of nanoindentation modulus of mineralized collagen⁵⁰ and mineralized collagen extracted from tooth dentin⁵¹—which has a nearly identical mineralization profile to native bone—and also closely approximate the values reported for mineralized bone collagen measured using AFM force spectroscopy (pulling) 52.

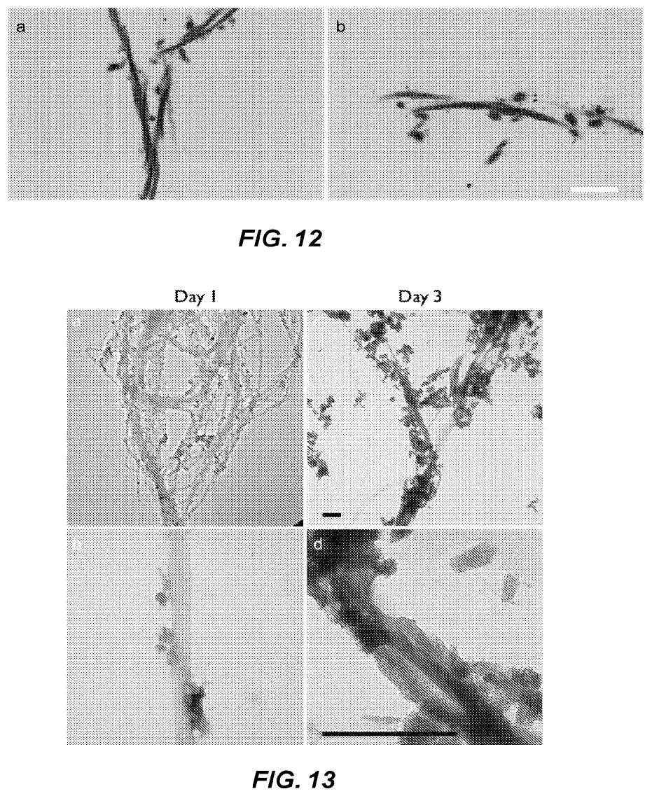

A number of studies have shed light on the conditions that are required for intrafibrillar mineralization of collagen. These earlier reports elucidated a complex system whereby non-collagenous proteins,^{15,62-64} water¹², and the arrangement of the tropocollagen molecules,² play an important role in dictating the formation of organized mineral crystallites within the fibrils, assisted by capillary forces¹⁴ and electrostatic/osmotic effects¹⁸. Different from these earlier studies, however, the methods and compositions disclosed herein provide a significant mixture of both intra- and extrafibrillar mineral, much like what is seen in the native bone tissue (FIG. 9). Without wishing to be bound by theory, this may be attributed to the fact that the disclosed mineralization protocols require the presence of serum proteins, vitamins and other cell culture medium components, which lead to the formation of mineral particles both within and on the surface of collagen fibrils (FIGS. 8, 11-13). These observations are consistent with previous studies that show the interference of different amino acids and proteins in the process of collagen biomineralization.⁶⁵

In the disclosed engineered bone-like tissue constructs, the regions adjacent to the embedded cells appeared more densely compacted with mineral clusters, when compared to the regions farther away from the cells (FIG. 2). This indicates that even though approximately 50% of the organic matrix was mineralized (FIG. 16), cells were still able to pull on and deform the surrounding matrix, hinting at active remodeling. Moreover, the cell bodies generated numerous processes that frequently protruded and retracted, indicating the effort to either probe or physically deform the surrounding mineralized matrix (FIG. 30). In fact, despite the increased local stiffness (FIG. 1k) and density of the hydrogel due to the formation of mineral crystallites, these results indicate that cells continued to spread, exhibiting numerous dendritic extensions that have strikingly similar morphology to maturing osteocytes⁵³ (FIGS. 2 and 3). A similar observation was noticed by Mata et al.⁵⁴, who described increased cell processes and osteoblastic differentiation in hole microtextures as opposed to groove microtextures, likely due to cells sensing these features as enclosed microenvironments with close cell-cell and cell-ECM contact. The formation of dendrite-like protrusions by cells could indicate a shift from a proteolytically degradation-driven process of cell spreading,⁵⁵ to a scenario where cells have to squeeze through the inter-fibrillar spaces between the stiffened fibers to make their way toward adjacent cells and establish cell-cell communication. This is consistent with previous reports that the formation of osteocyte processes is mainly triggered by intercellular separation and ECM mechanics⁵⁶. FIG. 2 panel d supports such a perspective, where narrow cell processes appear to protrude between crystals, rather than adhering onto and wrapping around them. Collectively, these observations may point to relevant morphological changes in stem cells as they respond to the gradually stiffening and calcifying bony microenvironment, that characterizes the transition from osteoid to mature, more heavily mineralized matrix. Another interesting observation in the disclosed mineralized samples was that after 7 days of culture, cells appeared to remodel their surroundings to form lacunae-like regions that had discrete pericellular spaces, and were bordered by cement-line boundaries (FIG. 3).²⁸ Overall, these results suggest that, when embedded in a microenvironment that replicates the three-dimensionality, composition and nanoscale structure of the mineralized bone niche, hMSCs expressed a multitude of morphological characteristics that are consistent with maturing bone cells, all in the absence of osteoinductive factors, and driven primarily by matrix mineralization.

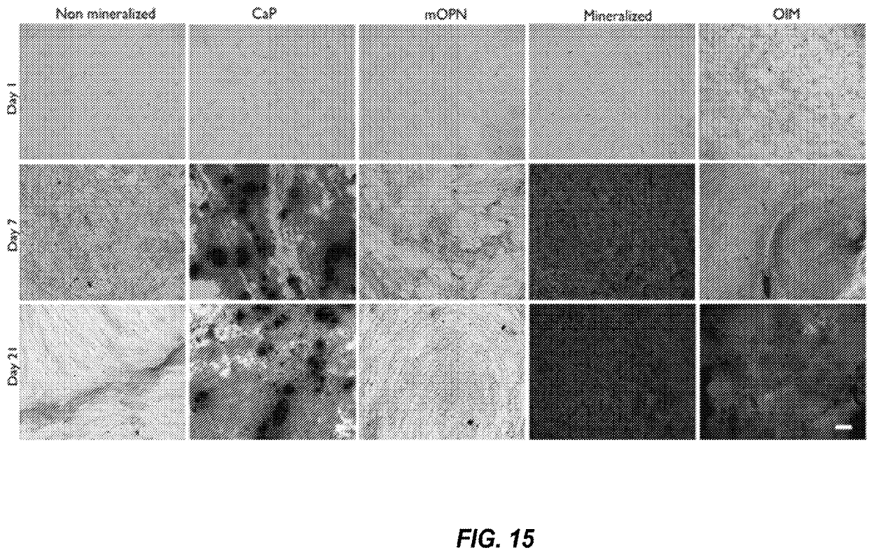

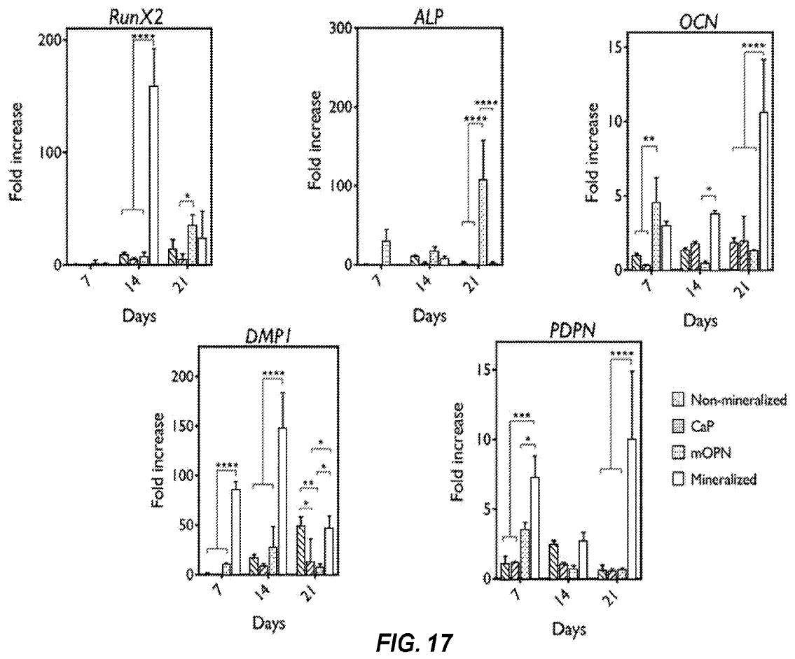

hMSCs are sensitive to a diverse array of microenvironmental cues.³⁸ Two factors in this proposed system that are known to influence stem cell differentiation are matrix stiffness and the presence of calcium and phosphate ions.^{38,57,58} The osteoinductive nature of calcium and phosphate scaffolds has long been attributed to their capability to modulate the extracellular concentrations of ionized Ca and P, that are sensed by cells either via Ras/Raf/ERK dependent signaling pathways,⁵⁸ or ATP-adenosine controlled mechanisms.⁵⁷ The results disclosed here showed that cell-laden mineralized hydrogels lead to a marked upregulation of several osteogenic genes (RUNX2, OCN, PDPN and DMP1) in comparison to non-mineralized controls (FIG. 4a). Interestingly, evidence suggests that calcium and phosphate substrates alone can be as potent as dexamethasone-supplemented medium in inducing osteogenic differentiation, 39 which is also consistent with the gene expression data disclosed here showing comparable or higher levels of mRNA between our mineralized constructs and the positive controls using osteoinductive medium. Supplementation of calcium and phosphate to the cell-laden constructs alone, however, did not have a significant effect on the expression of differentiation genes (FIG. 17), which suggests that bone-like apatite formation may be a pre-requisite for differentiation of hMSCs embedded in hydrogels cultured in the absence of growth factors and/or osteoinductive supplements. On the other hand, mOPN alone had a significant effect in increasing the expression of some osteogenic markers, which has been well reported in the literature.⁴⁰ However, the expression levels were never as high as in the mineralized samples or positive control, and such an increase was mostly temporary, given that mineralization was absent even after 21 days (FIG. 15).

The results disclosed herein also support the conjecture that matrix mineralization is associated with a concurrent elevation in the expression of pre-osteocytic markers, such as PDPN, a mucin-type glycoprotein required for the formation of dendritic processes in osteocytes;⁶⁶ and DMP1, a marker that has been shown to be predominantly expressed in chicken and rat osteocytes but not in osteoblasts.⁴¹ In fact, it is well known that mineralization of the surrounding matrix is a key determinant for osteoblast-to-osteocyte transition, both in-vitro⁴² and in-vivo.⁴³ Similarly, inhibition of mineral deposition has been linked to a decreased expression of PDPN, which further supports the role of matrix mineralization in driving osteocytogenesis.⁴² Generally, one would expect a significant upregulation in ALP production during early stages of osteoblastic differentiation, and a subsequent drop as cells mature into osteocytes. However, in the disclosed mineralized samples a reduction in ALP expression level was recorded from the earliest time point. Functionally, ALP stimulates mineral deposition by hydrolyzing inorganic pyrophosphate (PPi) to liberate inorganic phosphate (Pi), and studies suggest that a high extracellular Pi content can in turn inhibit ALP activity via a negative feedback mechanism,^{58,67} which is in agreement with these findings.

A key characteristic of native bone is the ability of resident cells, especially osteocytes, to regulate tissue homeostasis and remodeling in a paracrine fashion.^{30,44} The disclosed results indicate that the hMSCs embedded in mineralized microenvironments secrete significantly higher amounts of BMP-2 and BMP-6 compared to collagen alone (FIG. 4b). Interestingly, a contrasting effect seems to exist between mineralization- and osteoinductive medium mediated osteogenesis, wherein osteoinductive medium induces lower BMP-2 expression and increased ALP activity, as opposed to mineralization-driven osteogenesis, which resulted in a higher BMP-2 expression and lower ALP level. Also, since osteocytes play a significant role in osteoclast differentiation and recruitment, these cells are a major source of RANKL, a critical mediator of osteoclastogenesis.⁴⁶ During bone remodeling, an upregulation of RANKL is typically associated with a lower expression of OPG.⁴⁷ Such a ratio was greatly increased in the disclosed mineralized samples as compared with in osteoinductive medium-treated samples, thus demonstrating the ability of cells encapsulated in the mineralized matrix in potentially instructing osteoclastogenesis and active matrix remodeling in a paracrine manner.

Lastly, in addition to creating a 3D microenvironment that shares the key hallmarks of native bone extracellular matrix and inherent osteogenic potential, the proposed strategy successfully enables the recapitulation of the formation of hMSC-supported vascular capillaries (and innervation) (FIGS. 5a-5e and 6a-6d) prior to the onset of matrix mineralization, which is an important step toward the formation of functionally vascularized and innervated bone tissue models. Interestingly, the density and stability of these pre-mineralization engineered vessels was improved following in vivo implantation (FIG. 5e). One compelling view in the literature is the influence of hypoxia in regulating vessel integrity and tubulogenesis of endothelial cells by activating hypoxia-inducible factor (HIFs),⁵⁹ which may explain why hMSC-supported vascular capillaries were more stable and denser in mineralized samples than in non-mineralized controls after implantation. Overall, these observations point to an important characteristic of the proposed approach, where the ability to direct extracellular matrix mineralization in a controllable fashion, as shown in FIG. 32, allows one to stimulate the formation of hMSC-supported vasculature and innervation prior to the onset of mineral formation, without compromising the function of the formed vessels in vivo.

Although bone is a preferred site for prostate, breast and lung cancer metastasis, the precise molecular mechanisms driving the cross-talk between tumor cells and the bone microenvironment are poorly understood. This is mainly due to the lack of appropriate models that enable one to isolate the constituents of the bone microenvironment that trigger or enable such a response. Disclosed herein are methods that may recreate the bone microenvironment in vitro and the assessment of the ability of these engineered models to form a conducive milieu for ectopic colonization and growth of prostate cancer cells (PC3) in vivo. In the native bone, the paracrine signals exerted by the osteogenic cells, as well as matrix molecules released by the actively remodeling mineralized tissue act as crucial players in providing a conducive environment for the proliferation of tumor cells. The results disclosed herein confirm that the paracrine signaling from bone cells embedded in the tissue are critically important for colonization and growth of prostate cancer cells (PC3) in vivo, since a comparable rate of tumor growth was found between the mineralized samples (FIGS. 5f-5g) and collagen loaded with cells treated with an osteoinductive medium prior to implantation. Cells treated with mOPN alone, on the hand, had no effects on cancer progression in vivo. These results illustrate the ability of the proposed model system in dissecting the participation of different components in a process of disease progression and, potentially, response to treatment.

In summary, disclosed herein is a biomimetic approach for in-vitro engineering a bone-like model system that replicates the nanoscale mineralization of 3D bone microenvironments loaded with osteoprogenitor, vascular, and neural cells³³, leading to ultrastructural organization and composition that closely emulate that of native bone. The approach is also time-controllable, with the versatility of the synthesis being initiated and stopped at different time points (FIG. 32). Thus, the method and compositions disclosed herein may be used in the development of bone drugs, bone regeneration, and the understanding of bone physiology and disease.

### Examples

### Methods

The following examples are illustrative of disclosed methods and compositions. In light of this disclosure, those of skill in the art will recognize that variations of these examples and other examples of the disclosed methods and compositions would be possible without undue experimentation.

### Cell Culture

All experiments used mesenchymal stem cells isolated from human bone marrow. Cells were used from passages 2-4. Prior to experiments, cells were cultured in DMEM with 10% FBS, 1% L-Glutamine (200 mM) and 1% antibiotic solution. Similarly, human umbilical vein endothelial cells (HUVECs) expressing green fluorescent protein (Angioproteomie) were cultured in Endothelial Growth Media (EGM-Lonza). SH-SY5Y neuroblastoma cells (ATCC) were cultured in growth medium containing a mixture of DMEM and Ham's F-12 medium (1:1) supplemented with 10% FBS, 1% L-Glutamine (200 mM) and 1% antibiotic solution. Cells were maintained in culture flasks at 37° C. in a humidified atmosphere containing 5% CO₂ in air and sub-cultured using 0.25% trypsin-EDTA when cells reached 80-90% confluency.

### Cell-Laden Hydrogels

To prepare cell-laden collagen hydrogels, acid solubilized Type 1 collagen from rat tail tendon (3 mg/mL, BD Biosciences) was reconstituted in an ice bath to a final concentration of 1.5 mg/mL in 10×PBS along with DMEM containing a hMSC suspension of 5×10⁵ cells/mL. The pH was adjusted to 7.4 by neutralizing the hydrogel precursors with 1N NaOH. 100 μL of the gels were pipetted onto 24 well plates and were allowed to undergo fibrillogenesis in a humidified 5% CO₂ incubator at 37° C. for 30 min. For co-culture experiments, hUVECs and hMSCs were seeded at a ratio of 4:1 to a final concentration of 2.5×10⁶ cells/mL.

For pericyte supported endothelial tubulogenesis, hUVECs and hMSCs were encapsulated in collagen at a ratio of 4:1 to a final concentration of 2.5×10⁶ cells/mL, cultured for 3 days and the constructs were then mineralized. Likewise, for neurogenic induction, SH-SY5Y cells co-encapsulated with hMSCs (4:1) were pre-differentiated with 10 μM retinoic acid (RA) containing low serum medium for 7 days, followed by differentiation in neurobasal medium supplemented with a combination of B-27 supplement, 10 μM RA, 50 ng/ml brain-derived neurotrophic factor (BDNF), 1% FBS, 1% L-Glutamine (200 mM) and 1% antibiotic solution, for additional 7 days, after which the constructs were mineralized.

### Nanoscale Hydrogel Mineralization

In order to induce mineralization of collagen in the presence of cells, a modified mineralization medium was formulated by mixing equal volumes of 9 mM CaCl₂:2H₂O (J. T. Baker) and 4.2 mM K₂HPO₄ (J. T. Baker) in DMEM supplemented with 10% FBS. Osteopontin powder, extracted from milk (Arla Foods), was used at a concentration of 100 μg/mL to serve as the mineralization-directing agent, and was added in the CaCl₂ containing medium before the addition of K₂HPO₄. To ensure stable maintenance of pH at 7.4, 25 mM HEPES was added to the medium. The samples were incubated under continuous agitation in a rotary shaker so as to ensure uniform mineralization throughout the samples. The mineralizing medium was replenished every 24 hrs for the first 3 days to induce complete calcification of the collagen gels. Subsequently constructs were cultured using DMEM with 10% FBS without mineralization supplements for the rest of the culture period. Cell culture medium supplemented with a cocktail of osteoinductive factors containing dexamethasone (100 nM), ascorbic acid (50 μM) and β-glycerol phosphate (10 mM) were used as a positive control (denoted as OIM). For vascularization experiments involving co-culture of hUVECs with hMSCs, samples were cultured in DMEM-EGM-2 medium for 3 days, after which a mineralizing medium supplemented with EGM-2 Bullet Kit was used as described before. Alternatively, for innervation experiments involving co-culture of SH-SY5Y with hMSCs, the cells were subjected to neurogenic differentiation for 14 days, followed by 3 days exposure to mineralizing medium supplemented with a mixture of B-27 supplement, 10 UM RA, and 50 ng/ml BDNF.

### Reactive Oxidative Stress

Reactive oxygen species (ROS) were measured using a CM-H₂DCFDA (Abcam) kit to detect any cellular oxidative damage during the mineralization procedure. Measurements were performed immediately after mineralization and after 7 days of culture. Briefly, cell-laden hydrogels were stained in culture media with 20 μM 2′,7′-dichlorofluorescin diacetate (DCFDA) for 30 min at 37° C. The 2′,7′-dichlorofluorescein (DCF) fluorescence intensity was measured using a fluorescence microplate reader with excitation and emission at 485 nm and 535 nm, respectively. Tert-Butyl Hydrogen Peroxide was used as the positive control for detection of ROS (N=6).

### Proliferation Assay

hMSCs (5000 cells per hydrogel) were encapsulated within each non-mineralized, mineralized and OIM-treated hydrogels and cultured for durations of 1, 3, 7, 14 and 21 days. At the end of each of these time points, the culture medium was replaced with fresh medium containing 10% v/v AlamarBlue and the cells were allowed to incubate for 5 h. Subsequently, the formation of fluorescent resazurin products in aliquots of the culture medium was measured in 96-well plates using Tecan Infinite M200 Pro microplate reader (Tecan Trading AG) at excitation and emission wavelength of 550 nm and 590 nm respectively. The fluorescent readings were then correlated to the cell number by plotting a standard curve of known cell numbers over a range of 5×10³ to 8×10⁴. (N=6)

### Live & Dead

The viability of the cells encapsulated in hydrogels was determined using a live and dead assay kit (Molecular Probes). Cells were incubated for 10 min, followed by rinsing in PBS and imaging using an inverted fluorescence microscope (FL Auto, Evos). Live and dead cell numbers were counted using ImageJ and the percentage of viable cells was quantified as the number of live cells divided by the total cell number (N=6).

### FTIR Analysis

FTIR spectra were obtained in transmission mode (Nicolet 6700, Thermo Scientific). Using 32 scans in the range of 4000 to 400 cm⁻¹ at a resolution of 4 cm⁻¹. The mineral to matrix ratio was calculated from the area of v³PO4 (1030 cm⁻¹) over amide (1660 cm⁻¹) peaks after baseline correction and normalization. The crystallinity index was calculated from the parameter splitting factor corresponding to the doublet peak in the fingerprint region (500-650 cm⁻¹) that is attributed mainly to v⁴PO₄³⁻ bending vibrations. The parameter is calculated as the sum of the peak heights at 565 cm⁻¹ and 605 cm⁻¹ divided by the height of the minimum between this doublet at 590 cm⁻¹. All the height measurements were performed using Origin 8.0 software after baseline correction and normalization of the spectra to the intensity of amide I band (1585-1720 cm-1) (N=6).

### Electron Microscopy

Scanning electron microscopy. For SEM analysis, samples were fixed with 2.5% glutaraldehyde for 1 h at room temperature, washed in distilled water and subjected to a series of ethanol dehydration steps for 10 min each. Subsequently, the samples were critical point dried, sputter coated with gold/palladium and observed under SEM (FEI Helios Nanolab™ 660 DualBeam™) (N=6). The elemental analysis for the presence of Ca and P was carried out using the attached EDX detector (energy dispersive X-ray spectroscopy; INCA, Oxford Instruments) (N=4).

Transmission electron microscopy. For TEM imaging, both mineralized and non-mineralized hydrogels were minced with a double-edge razor blade and were immersed in ice-cold 0.1M ammonium bicarbonate (pH 7.8). While on ice, the minced hydrogels were then exposed to the cutting blades of an OMNI 2000 tissue homogenizer (OMNI International, Kennesaw, GA) operated at approximately 11,700×g until no visible fragments remained. The homogenate was then pipetted onto freshly glow-discharged 600 mesh carbon coated TEM grids and observed directly using FEI G20 TEM operated at 120 kV.

For tilt-series electron tomography analysis, the homogenized hydrogels were exposed overnight at 4C to 1.5% glutaraldehyde/1.5% formaldehyde with 0.05% tannic acid, then dehydrated and embedded in Spurrs epoxy. Following, 450 nm thick sections were cut with a diamond knife using a Leica EM UC7 ultramicrotome and mounted on formvar coated 1×2 mm slot grids. Sections were subsequently stained in uranyl acetate and lead citrate and imaged at 200 kV using FEI G20 TEM. For 3D tilt series, 450 nm thick sections were imaged at 2 degree increments between 0 and 40 degrees, then at 1 degree increments between 40 and 70 degrees, then identically imaged from 0 to −40 degrees and −40 to −70 degrees. Tilt series images were collected using FEI Eagle camera directed by FEI Tomography software, then aligned using FEI “Inspect 3D” software (N=6). For selected area electron diffraction (SAED) analysis, samples were freeze dried in liquid nitrogen and placed between two lacey carbon TEM grids and imaged using a TECNAI F20 TEM with an Oxford SDD EDS detector and Gatan GIF 2001 system operated at 200 kV. (N=4)

For Serial Block Face-Scanning electron microscopy (SBF-SEM), samples were fixed in Karnovsky's fixative overnight, followed by microwave assisted embedding process using BioWave Pelco Microwave. Briefly, after washing with 0.1 M cacodylate buffer, the samples were successively post-fixed in 1% osmium tetroxide containing 1.5% potassium ferrocyanide in 0.1 M cacodylate buffer and then immersed in 1% tannic acid, followed by 2% aqueous osmium tetroxide and finally staining in 1% aqueous uranyl acetate. The samples were then rinsed, dehydrated with a graded series of acetone and were subsequently embedded in Epon resin. The resin embedded samples were sputter coated with platinum/palladium. A series of block face images were obtained using a Scanning electron microscope (Teneo Volumescope™, FEI) equipped with an in-chamber ultramicrotome (N=3). A sequence of images was acquired every 60 nm depth with a backscattered electron detector at an acceleration voltage of 2.7 kV under high vacuum. Selected serial thin section images were then loaded into an image analyses software (Amira) and processed using a 3D reconstruction plug-in (DualBeam 3D Wizard). The segmentation of cells was done by manually outlining the cell borders, whereas the high contrast difference between the mineral and the non-mineralized collagen was distinguished using a threshold tool. The segmented data sets were further volume-rendered and animated using the Amira Animation Director tool (N=3).

### Confocal Imaging

A laser-scanning confocal microscope (Zeiss LSM 880) was used for immunofluorescence and reflectance imaging. Briefly, for the immunofluorescent staining, samples were fixed with 4% paraformaldehyde and permeabilized using 0.1% Triton X-100 (N=3). The constructs were further blocked using 1.5% bovine serum albumin (BSA) in PBS for 1 h, followed by incubation with Image-iT FX signal enhancer (Invitrogen, CA) for 30 min to remove background staining. Cells were then incubated with primary antibodies overnight at 4° C., as listed below. Subsequently, cells were washed three times with PBS/0.1% Tween-20 and incubated with secondary antibodies overnight at 4° C. The following primary antibodies were used: rabbit polyclonal anti-osteocalcin (Bioss antibodies) (1:50 dilution), mouse monoclonal anti-PDPN (Origene) (1:100 dilution), mouse monoclonal anti-CD31 (Dako) (1:200 dilution), rabbit polyclonal anti-RUNX2/CBFA1 antibody (Novus Biologicals) (1:100 dilution), mouse monoclonal alpha smooth muscle actin (αSMA) (Invitrogen) (1:400 dilution), mouse monoclonal anti-NEFL antibody (Thermo Fisher Scientific) (1:50 dilution), and mouse monoclonal anti-Neuron-specific enolase antibody (Abcam) (1:1000 dilution). The following secondary antibodies were used at the specified dilutions: Alexa Flour 555 goat anti-mouse IgG (Thermo Fisher Scientific) (1:200 dilution), Alexa Fluor 647 goat anti-rabbit IgG (Thermo Fisher Scientific) (1:200 dilution).

The F-actin was visualized by staining with Alexa Fluor 488 conjugated phalloidin and the nucleus was stained with 4′,6-diamidino-2-phenylindole (DAPI). For reflectance imaging of collagen fibrils, the microscope was configured to capture the reflected light between 485 nm and 495 nm, after exciting with a 514 nm laser. 3D reconstructions of z-stacks of samples were processed and rendered on ZEN black (Zeiss) and Imaris 8 (Bitplane) software. For the quantification of vessel parameters, the images were analyzed using AngioTool (National Cancer Institute, NIH). Likewise, neuronal morphometric analysis was performed using Imaris Filament Tracer module.

### Atomic Force Microscopy

The nanomechanical properties of individual non-mineralized and mineralized collagen fibrils were investigated using a Nanoscope 8 atomic force microscope (J scanner, Bruker) in PeakForce tapping mode. The indentation measurements were performed both in the hydrated state and in air. Al-coated, silicon AFM tips of 300 KHz resonance frequency, 26 N/m nominal spring constant and a tip curvature radius of ˜10 nm (AC160TS; Olympus) were used for non-mineralized collagen fibril tested in air and for mineralized collagen fibril measured in air and in water. On the other hand, Au-coated Si₃N₄ AFM tips of 65 kHz resonance frequency, 0.35 N/m nominal spring constant and a tip curvature radius of ˜30 nm (DNP-S, triangle A, Bruker) were used for non-mineralized collagen fibrils in water. These specific cantilevers were chosen to match the stiffness of collagen or mineralized collagen for optimizing the sensitivity. The spring constant of the cantilever was calibrated by the thermal tuning method (Mullen et al., Osteocyte differentiation is regulated by extracellular matrix stiffness and intercellular separation. Journal of the mechanical behavior of biomedical materials 28, 183-194, doi: 10.1016/j.jmbbm.2013.06.013 (2013)). After acquiring the 2D topographic image of the fibril, the load-displacement curves at 5-12 randomly selected spots on mica and on the fibril selected were collected under quasi-static indentations. The loading-unloading rate was set to be 100 nm/s, with zero delays in-between. The elastic modulus was obtained by performing Hertz fits of the indentation force against depth curves, as described previously. (Shih et al., Proc Nat Acad Sci 111, 990 (2014)⁵⁷; Barradas et al. Biomaterials 33, 3205-3215, doi: 10.1016/j.biomaterials.2012.01.020 (2012)⁵⁸; and Covello et al., Current topics in developmental biology 62, 37-54, doi: 10.1016/s0070-2153 (04) 62002-3 (2004)⁵⁹ (N=3).

### Real Time PCR

Total RNA from hMSCs were isolated using Tri reagent (Zymogen, USA) according to the manufacturer's instructions. After determining the purity and concentration of the extracted RNA by Nanodrop (Thermo Scientific, USA), complementary DNA was reverse transcribed from 1 μg RNA using SuperScript III first-strand synthesis system (Invitrogen). Quantitative PCR was performed using Power SYBR® Green PCR Master Mix with the following cycling conditions: Pre-incubation at 95° C. for 10 min; 40 cycles of denaturation at 95° C. for 30 s, annealing at 50-60° C. for 30 s; and extension at 95° C. for 30 s, followed by melt curve analysis to validate the specificity of PCR products. The sequences of the primer set used for the study is provided in Table 1. The specified primers were designed using Primer 3 software and blasted against GenBank database sequences to achieve high specificity primers. GAPDH was used as the internal reference gene for normalization. The fold change in the expression of each gene was calculated using the 2^{-ΔΔct} method (N=3).

TABLE 1

| Gene | |
|---|---|
| code | Nucleotide Sequence (5′ - 3′) |
| ALP | For: ACATTCCCACGTCTTCACATTT (SEQ ID NO: 1) |
| | Rev: AGACATTCTCTCGTTCACCGCC (SEQ ID NO: 2) |
| OCN | For: TGTGAGCTCAATCCGGACTGT (SEQ ID NO: 3) |
| | Rev: CCGATAGGCCTCCTGAAGC (SEQ ID NO: 4) |
| RUNX2 | For: AGATGATGACACTGCCACCTCTG (SEQ ID NO: 5) |
| | Rev: GGGATGAAATGCTTGGGAACT (SEQ ID NO: 6) |
| DMP1 | For: AGAAGCGAGCTTGATGACAACAA (SEQ ID NO: 7) |
| | Rev: TGGACTCACTGCTGGGACCATCTAC (SEQ ID NO: 8) |
| PDPN | For: TTACTAGCCATCGGCTTCATT (SEQ ID NO: 9) |
| | Rev: GGCGAGTACCTTCCCGACAT (SEQ ID NO: 10) |
| GAPDH | For: AACAGCGACACCCACTCCTC (SEQ ID NO: 11) |
| | Rev: CATACCAGGAAATGAGCTTGACAA (SEQ ID NO: 12) |

### Antibody Array Profiling for Protein Expression

Bone metabolism associated proteins/cytokines were quantified using a multiplex ELISA array (Human Bone Metabolism Array Q2; Raybiotech), according to manufacturer's instructions. Proteins were isolated either from cell lysates or conditioned medium after 14 days and stored at −80° C. until use. Subsequently, samples were incubated in an array chip printed with capture antibodies of interest. The chips were then incubated with biotinylated detection antibody cocktail, followed by incubation in Cy3-labeled Streptavidin. The slides were then scanned using a gene microarray laser scanner and the signal intensities were detected using densitometric analysis to semi-quantitatively measure the protein level (N=5).

### In Vivo Implantation

Subcutaneous implantation was performed in 5-7 weeks old, female SCID beige mice (Charles River Laboratories), after approval from the institutional animal research ethics committee. The samples were randomized and implanted into four separate sites on the back of each mouse (N=6). One week after implantation, the samples were removed, fixed in 10% neutral buffered formalin and embedded in paraffin. The embedded samples were then sectioned (5 μm thick) and stained using hematoxylin and eosin, von Kossa, Masson's Trichrome, human-specific CD31 monoclonal antibody (1:250; company name), anti-αSMA (1:800; company name, recognizes both mouse and human αSMA), and Rabbit polyclonal anti-DMP1 (Invitrogen) (1:100 dilution). Secondary antibody staining was performed using horseradish peroxidase-conjugated anti-rabbit/mouse IgG antibody and the peroxidase activity was detected using the 3,3-diaminobenzidine (DAB) detection system. All the immunostained sections were counterstained with Mayer's haematoxylin (Sigma-Aldrich). The whole slides were then digitized using a Zeiss AxioScan Z1 Slide scanner at ×20 objective. Further, the total number of vessels per field were determined by counting the CD31⁺ vessels within the construct. Similarly, the diameter of the CD31 positive vessels were manually quantified using ImageJ. The percentage area of CD31⁺ and αSMA⁺ staining was quantified using a color deconvolution plug-in, followed by threshold setting and automated quantification of the immunostained area fraction by ImageJ. Mean±SD values presented for each experimental group correspond to the average values obtained from at least 3 animals per group.

To study the interaction of prostate cancer cells with our engineered bone model, the constructs were subcutaneously implanted on the left and right dorsal flanks of 6-8 weeks old male athymic mice (2 constructs/animal). One day post implantation, luciferase-expressing PC3 cells (100,000) (ATCC) suspended in 100 ul PBS were injected directly to the target site. The growth rate of PC3 cells was subsequently monitored weekly for up to 3 weeks using an IVIS Spectrum in-vivo imaging system (Perkin Elmer). Mice were given an intraperitoneal injection of 150 mg/kg D-luciferin dissolved in PBS and the emitted luminescence was analyzed using Living Image 4.3 software (Perkin Elmer). The signal intensity expressed as total flux (photons/second) was quantified as the sum of all detected photon flux counts from the region of interest manually drawn around the tumor during data post processing.

### Statistical Analysis

For the experiments involving the comparison of two groups, statistical analysis was performed using two-tailed, unpaired Student t-test (Prism5, GraphPad Software). For experiments involving more than two groups, one-way or two-way analysis of variance (ANOVA) with Tukey's post hoc test for multiple comparisons was used to identify significant differences. A p value lower than 0.05 was considered statistically significant. All the quantitative data is presented as mean±SD.

Numerous references have been made to patents and printed publications throughout this specification. Each of the above-cited references and printed publications are individually incorporated herein by reference in their entirety.

It is to be understood that the embodiments of the present disclosure are illustrative of the principles of the present disclosure. Other modifications that may be employed are within the scope of the disclosure. Thus, by way of example, but not of limitation, alternative configurations of the present disclosure may be utilized in accordance with the teachings herein. For example, groupings of alternative elements or embodiments of the invention disclosed herein are not to be construed as limitations. Each group member may be referred to and claimed individually or in any combination with other members of the group or other elements found herein. Accordingly, the present disclosure is not limited to that precisely as shown and described.

It will be apparent to those having skill in the art that many changes may be made to the details of the above-described embodiments without departing from the underlying principles of the invention. The scope of the present invention should, therefore, be determined only by the following claims.

### REFERENCES

1. Reznikov, N., Steele, J. A. M., Fratzl, P. & Stevens, M. M. A materials science vision of extracellular matrix mineralization. Nat. Rev. Mater. 1, 16035-16041 (2016).
2. Nudelman, F. et al. The role of collagen in bone apatite formation in the presence of hydroxyapatite nucleation inhibitors. Nat. Mater. 9, 1004-1009 (2010).
3. Reznikov, N., Bilton, M., Lari, L., Stevens, M. M. & Kröger, R. Fractal-like hierarchical organization of bone begins at the nanoscale. Science 360, 1-10 (2018).
4. Landis, W. J., Song, M. J., Leith, A., McEwen, L. & McEwen, B. F. Mineral and organic matrix interaction in normally calcifying tendon visualized in three dimensions by high-voltage electron microscopic tomography and graphic image reconstruction. J. Struct. Biol. 110, 39-54 (1993).
5. Traub, W., Arad, T. & Weiner, S. Origin of mineral crystal growth in collagen fibrils. Matrix (Stuttg., Ger.) 12, 251-255 (1992).
6. Nair, A. K., Gautieri, A., Chang, S.-W. & Buehler, M. J. Molecular mechanics of mineralized collagen fibrils in bone. Nat. Commun. 4, 1724 (2013).
7. Weiner, S. & Wagner, H. D. The material bone: structure-mechanical function relations. Annu. Rev. Mater. Sci. 28, 271-298 (1998).
8. He, G., Dahl, T., Veis, A. & George, A. Nucleation of apatite crystals in vitro by self-assembled dentin matrix protein 1. Nat. Mater. 2, 552-558 (2003).
9. Gorski, J. P. Biomineralization of bone: a fresh view of the roles of non-collagenous proteins. Front. Biosci. (Landmark Ed.) 16, 2598-2621 (2011).
10. Wang, Y. et al. The predominant role of collagen in the nucleation, growth, structure and orientation of bone apatite. Nat. Mater. 11, 724-733 (2012).
11. Newcomb, C. J., Bitton, R., Velichko, Y. S., Snead, M. L. & Stupp, S. I. The role of nanoscale architecture in supramolecular templating of biomimetic hydroxyapatite mineralization. Small 8, 2195-2202 (2012).
12. Wang, Y. et al. Water-mediated structuring of bone apatite. Nat. Mater. 12, 1144-1153 (2013).
13. Zhao, W., Xu, Z., Yang, Y. & Sahai, N. Surface energetics of the hydroxyapatite nanocrystal-water interface: a molecular dynamics study. Langmuir 30, 13283-13292 (2014).
14. Nudelman, F., Bomans, P. H., George, A., de With, G. & Sommerdijk, N. A. The role of the amorphous phase on the biomimetic mineralization of collagen. Faraday Discuss. 159, 357-370 (2012).
15. Mahamid, J. et al. Mapping amorphous calcium phosphate transformation into crystalline mineral from the cell to the bone in zebrafish fin rays. Proc. Natl Acad. Sci. USA 107, 6316-6321 (2010).
16. Olszta, M. J. et al. Bone structure and formation: a new perspective. Mater. Sci. Eng. R Rep. 58, 77-116 (2007).
17. Xu, Z. et al. Molecular mechanisms for intrafibrillar collagen mineralization in skeletal tissues. Biomaterials 39, 59-66 (2015).
18. Niu, L.-n et al. Collagen intrafibrillar mineralisation as a result of the balance between osmotic equilibrium and electroneutrality. Nat. Mater. 16, 370-378 (2017).
19. Morin, C. & Hellmich, C. Mineralization-driven bone tissue evolution follows from fluid-to-solid phase transformations in closed thermodynamic systems. J. Theor. Biol. 335, 185-197 (2013).
20. Liu, Y. et al. Intrafibrillar collagen mineralization produced by biomimetic hierarchical nanoapatite assembly. Adv. Mater. 23, 975-980 (2010).
21. Hartgerink, J. D., Beniash, E. & Stupp, S. I. Self-assembly and mineralization of peptide-amphiphile nanofibers. Science 294, 1684-1688 (2001).
22. Spoerke, E. D., Anthony, S. G. & Stupp, S. I. Enzyme directed templating of artificial bone mineral. Adv. Mater. 21, 425-430 (2009).
23. Elsharkawy, S. et al. Protein disorder-order interplay to guide the growth of hierarchical mineralized structures. Nat. Commun. 9, 2145 (2018).
24. Stupp, S. I., Mejicano, G. C. & Hanson, J. A. Organoapatites: Materials for artificial bone. II. Hardening reactions and properties. J. Biomed. Mater. Res. 27, 289-299 (1993).
25. Thula, T. T. et al. In vitro mineralization of dense collagen substrates: a biomimetic approach toward the development of bone-graft materials. Acta Biomater. 7, 3158-3169 (2011).
26. Tang, Z., Li, X., Tan, Y., Fan, H. & Zhang, X. The material and biological characteristics of osteoinductive calcium phosphate ceramics. Regen. Biomater. 5, 43-59 (2018).
27. Murphy, C. M., O'Brien, F. J., Little, D. G. & Schindeler, A. Cell-scaffold interactions in the bone tissue engineering triad. Eur. Cells Mater. 26, 120-132 (2013).
28. Shin, K., Acri, T., Geary, S. & Salem, A. K. Biomimetic mineralization of biomaterials using simulated body fluids for bone tissue engineering and regenerative medicine. Tissue Eng. Part A 23, 1169-1180 (2017).
29. O'Brien, F. J. Biomaterials and scaffolds for tissue engineering. Mater. Today 14, 88-95 (2011).
30. Bonewald, L. F. The amazing osteocyte. J. Bone Miner. Res. 26, 229-238 (2011).
31. Kretlow, J. D. & Mikos, A. G. Review: mineralization of synthetic polymer scaffolds for bone tissue engineering. Tissue Eng. 13, 927-938 (2007).
32. Annabi, N. et al. Rational design and applications of hydrogels in regenerative medicine. Adv. Mater. 26, 85-124 (2014).
33. Gentleman, E. et al. Comparative materials differences revealed in engineered bone as a function of cell-specific differentiation. Nat. Mater. 8, 763 (2009).
34. Rodriguez, D. E. et al. Multifunctional role of osteopontin in directing intrafibrillar mineralization of collagen and activation of osteoclasts. Acta Biomater. 10, 494-507 (2014).
35. Rubel, J. R. & Milford, E. L. The relationship between serum calcium and phosphate levels and cardiac valvular procedures in the hemodialysis population. Am. J. Kidney Dis. 41, 411-421 (2003).
36. Boonrungsiman, S. et al. The role of intracellular calcium phosphate in osteoblast-mediated bone apatite formation. Proc. Natl Acad. Sci. USA 109, 14170 (2012).
37. Hsieh, W.-T. et al. Matrix dimensionality and stiffness cooperatively regulate osteogenesis of mesenchymal stromal cells. Acta Biomater. 32, 210-222 (2016).
38. Lutolf, M. P., Gilbert, P. M. & Blau, H. M. Designing materials to direct stem-cell fate. Nature 462, 433 (2009).
39. Viti, F. et al. Osteogenic differentiation of MSC through calcium signaling activation: transcriptomics and functional analysis. PLOS ONE 11, e0148173 (2016).
40. Chen, Q. et al. An osteopontin-integrin interaction plays a critical role in directing adipogenesis and osteogenesis by mesenchymal stem cells. Stem Cells (Dayt., Ohio) 32, 327-337 (2014).
41. Toyosawa, S. et al. Dentin matrix protein 1 is predominantly expressed in chicken and rat osteocytes but not in osteoblasts. J. Bone Miner. Res. 16, 2017-2026 (2001).
42. Prideaux, M., Loveridge, N., Pitsillides, A. A. & Farquharson, C. Extracellular matrix mineralization promotes E11/gp38 glycoprotein expression and drives osteocytic differentiation. PLOS ONE 7, e36786 (2012).
43. Irie, K., Ejiri, S., Sakakura, Y., Shibui, T. & Yajima, T. Matrix mineralization as a trigger for osteocyte maturation. J. Hisctochem. Cytochem. Soc. 56, 561-567 (2008).
44. Xiong, J. et al. Matrix-embedded cells control osteoclast formation. Nat. Med. 17, 1235 (2011).
45. Bonewald, L. F. Osteocytes as dynamic multifunctional cells. Ann. New Y. Acad. Sci. 1116, 281-290 (2007).
46. Xiong, J. & O'Brien, C. A. Osteocyte RANKL: new insights into the control of bone remodeling. J. Bone Miner. Res. 27, 499-505 (2012).
47. Grimaud, E. et al. Receptor activator of nuclear factor KB ligand (RANKL)/osteoprotegerin (OPG) ratio is increased in severe osteolysis. Am. J. Pathol. 163, 2021-2031 (2003).
48. Bonewald, L. F. Generation and function of osteocyte dendritic processes. J. Musculoskelet. Neuron. Interact. 5, 321-324 (2005).
49. Skedros, J. G., Holmes, J. L., Vajda, E. G. & Bloebaum, R. D. Cement lines of secondary osteons in human bone are not mineral-deficient: new data in a historical perspective. Anat. Rec. A Discov. Mol. Cell. Evol. Biol. 286, 781-803 (2005).
50. Grant, C. A., Brockwell, D. J., Radford, S. E. & Thomson, N. H. Tuning the elastic modulus of hydrated collagen fibrils. Biophys. J. 97, 2985-2992 (2009).
51. Balooch, M., Habelitz, S., Kinney, J. H., Marshall, S. J. & Marshall, G. W. Mechanical properties of mineralized collagen fibrils as influenced by demineralization. J. Struct. Biol. 162, 404-410 (2008).
52. Hang, F. & Barber, A. H. Nano-mechanical properties of individual mineralized collagen fibrils from bone tissue. J. R. Soc. Interface 8, 500-505 (2011).
53. Kamioka, H., Honjo, T. & Takano-Yamamoto, T. A three-dimensional distribution of osteocyte processes revealed by the combination of confocal laser scanning microscopy and differential interference contrast microscopy. Bone 28, 145-149 (2001).
54. Mata, A. et al. Micropatterning of bioactive self-assembling gels. Soft Matter 5, 1228-1236 (2009).
55. Linder, S., Wiesner, C. & Himmel, M. Degrading devices: invadosomes in proteolytic cell invasion. Annu. Rev. Cell Dev. Biol. 27, 185-211 (2011).
56. Mullen, C. A., Haugh, M. G., Schaffler, M. B., Majeska, R. J. & McNamara, L. M. Osteocyte differentiation is regulated by extracellular matrix stiffness and intercellular separation. J. Mech. Behav. Biomed. Mater. 28, 183-194 (2013).
57. Shih, Y.-R. V. et al. Calcium phosphate-bearing matrices induce osteogenic differentiation of stem cells through adenosine signaling. Proc. Natl Acad. Sci. USA 111, 990 (2014).
58. Barradas, A. M. et al. A calcium-induced signaling cascade leading to osteogenic differentiation of human bone marrow-derived mesenchymal stromal cells. Biomaterials 33, 3205-3215 (2012).
59. Covello, K. L. & Simon, M. C. HIFs, hypoxia, and vascular development. Curr. Top. Dev. Biol. 62, 37-54 (2004).
60. Gorski, J. P. Biomineralization of bone: a fresh view of the roles of non-collagenous proteins. Frontiers in bioscience (Landmark edition) 16, 2598-2621 (2011).
61. Pidaparti, R. M., Chandran, A., Takano, Y. & Turner, C. H. Bone mineral lies mainly outside collagen fibrils: predictions of a composite model for osteonal bone. Journal of biomechanics 29, 909-916 (1996).
62. He, G. et al. Spatially and temporally controlled biomineralization is facilitated by interaction between self-assembled dentin matrix protein 1 and calcium phosphate nuclei in solution. Biochemistry 44, 16140-16148 (2005).
63. Hunter, G. K. & Goldberg, H. A. Nucleation of hydroxyapatite by bone sialoprotein. Proceedings of the National Academy of Sciences of the United States of America 90, 8562-8565 (1993).
64. George, A. & Veis, A. Phosphorylated proteins and control over apatite nucleation, crystal growth, and inhibition. Chemical reviews 108, 4670-4693 (2008).
65. Tavafoghi, M. & Cerruti, M. The role of amino acids in hydroxyapatite mineralization. Journal of the Royal Society Interface 13, 20160462 (2016).
66. Zhang, K. et al. E11/gp38 selective expression in osteocytes: regulation by mechanical strain and role in dendrite elongation. Molecular and cellular biology 26, 4539-4552 (2006).
67. Sage, A. P., Lu, J., Tintut, Y. & Demer, L. L. Hyperphosphatemia-induced nanocrystals upregulate the expression of bone morphogenetic protein-2 and osteopontin genes in mouse smooth muscle cells in vitro. Kidney international 79, 414-422 (2011).
68. Olszta, M. J., Odom, D. J., Douglas, E. P., Gower, L. B. A new paradigm for biomineral formation: mineralization via amorphous liquid-phase precursor. Connective Tissue Res. 44:1, 326-334 (2003).

1. A three-dimensional (3D) cell-encapsulated matrix composition comprising:
(a) a mineralizing solution, the mineralizing solution being supersaturated with respect to one or more crystallizable metals and having a pH from about 6.0 to about 8.0, in which the crystallizable metals comprise alkali metals, earth alkali metals, or both, including ionic calcium, ionic phosphorus, or both;
(b) a buffering agent having a pH buffering range of about 6.0 to about 8.0;
(c) living cells;
(d) a 3D cell-encapsulated scaffold comprising hydrogel with the living cells configured to undergo directed mineralization to mimic native bone nanoscale structure;
(e) a basal medium comprising nutrients, buffering agents, and growth factors for supporting the growth of the living cells; and
(f) a nucleation inhibitor in an amount effective to modulate ions of the crystallizable metals, reducing cytotoxic effects on the living cells by substantially controlling against spontaneous precipitation.

2. The composition of claim 1, in which the living cells comprise mammalian cells.

3-5. (canceled)

6. The composition of claim 1, in which the 3D cell-encapsulated scaffold comprises collagen.

7. (canceled)

8. The composition of claim 6, in which the collagen is fibrillated.

9-10. (canceled)

11. The composition of claim 1, in which the mineralizing solution comprises from about 3.0 mM to about 6.0 mM of ionic calcium.

12. The composition of claim 1, in which the mineralizing solution comprises from about 1.5 mM to about 3.0 mM of ionic phosphorus.

13. The composition of claim 1, in which the nucleation inhibitor inhibits nucleation or precipitation, or both, of hydroxyapatite.

14. The composition of claim 1, in which the nucleation inhibitor comprises Osteopontin, Osteocalcin, Osteonectin, bone sialoprotein, dentine phosphoryn, dentin matrix protein 1, dentin sialophosphoprotein (DSPP), matrix extracellular phosphoglycoprotein, chondrocalcin, proline-rich proteins such as Proline-rich protein 1, Proline-rich protein 2, and Proline-rich protein3, PRP1-T1, PRP3-T1, Histatin 5, MG1, MG2, Asialo_MG2, Amylase, statherin, cystatin S, cystatin SN, Cystatin S1, fetuin, HSA, poly-L-aspartic acid, or combinations thereof.

15. The composition of claim 1, in which the nucleation inhibitor comprises osteopontin from about 50 μg/mL to about 1000 μg/mL or about 50 μg/mL to about 150 μg/mL.

16. The composition of claim 1, in which the pH of the mineralizing solution is from about 7.2 to about 7.6.

17. A method of selectively mineralizing a three-dimensional (3D) cell-encapsulated matrix comprising:
providing a 3D cell-encapsulated matrix having a 3D cell-capsulated hydrogel scaffold with the living cells configured to undergo directed mineralization to mimic native bone nanoscale structure;
providing a mineralizing solution comprising:
a supersaturated solution with respect to one or more crystallizable metals in which the crystallizable metals comprise alkali metals, earth alkali metals, or both, including ionic calcium, ionic phosphorus, or both; and
a nucleation inhibitor in an amount effective to modulate ions of the crystallizable metals, reducing cytotoxic effects on the living cells by substantially controlling against spontaneous precipitation; and
exposing the 3D cell-encapsulated matrix to the mineralizing solution for a period to achieve a selected mineralization level.

18. The method of claim 17, in which the 3D cell-encapsulated matrix comprises mammalian cells.

19-21. (canceled)

22. The method of claim 17, in which the 3D cell-capsulated hydrogel scaffold comprises collagen.

23. (canceled)

24. The method of claim 22, further comprising fibrillating the collagen by adjusting the pH of the 3D cell-encapsulated matrix to about 6.0 to about 8.0 and maintaining its temperature from about 34° C. to about 40° C. until the collagen chains undergo fibrillogenesis.

25. (canceled)

26. The method of claim 17, in which the mineralizing solution comprises from about 3.0 mM to about 6.0 mM of ionic calcium.

27. The method of claim 17, in which the mineralizing solution comprises from about 1.5 mM to about 3.0 mM of ionic phosphorus.

28. The method of claim 17, in which the nucleation inhibitor inhibits nucleation or precipitation, or both, of hydroxyapatite.

29. The method of claim 17, in which the nucleation inhibitor comprises: Osteopontin, Osteocalcin, Osteonectin, bone sialoprotein, dentine phosphoryn, dentin matrix protein 1, dentin sialophosphoprotein (DSPP), matrix extracellular phosphoglycoprotein, chondrocalcin, proline-rich proteins such as Proline-rich protein 1, Proline-rich protein 2, and Proline-rich protein3, PRP1-T1, PRP3-T1, Histatin 5, MG1, MG2, Asialo_MG2, Amylase, statherin, cystatin S, cystatin SN, Cystatin S1, fetuin, HSA, or combinations thereof.

30. The method of claim 29, in which the nucleation inhibitor comprises osteopontin from about 50 μg/mL to about 150 μg/mL.

31. The method of claim 17, in which the period of exposing the 3D cell-encapsulated matrix to the mineralizing solution is about 1 minute to about 7 days.

32. The method of claim 31, in which the period is about 10 minutes to about 60 minutes.

33. The method of claim 17, in which the pH of the mineralizing solution is from about 7.2 to about 7.6.

34-43. (canceled)


SEQUENCE LISTING (12 sequences)

SEQ ID NO: 1 - DNA, 22 bp, Synthetic construct
  source 1..22: mol_type = other DNA
acattcccacgtcttcacattt

SEQ ID NO: 2 - DNA, 22 bp, Synthetic construct
  source 1..22: mol_type = other DNA
agacattctctcgttcaccgcc

SEQ ID NO: 3 - DNA, 21 bp, Synthetic construct
  source 1..21: mol_type = other DNA
tgtgagctcaatccggactgt

SEQ ID NO: 4 - DNA, 19 bp, Synthetic construct
  source 1..19: mol_type = other DNA
ccgataggcctcctgaagc

SEQ ID NO: 5 - DNA, 23 bp, Synthetic construct
  source 1..23: mol_type = other DNA
agatgatgacactgccacctctg

SEQ ID NO: 6 - DNA, 21 bp, Synthetic construct
  source 1..21: mol_type = other DNA
gggatgaaatgcttgggaact

SEQ ID NO: 7 - DNA, 23 bp, Synthetic construct
  source 1..23: mol_type = other DNA
agaagcgagcttgatgacaacaa

SEQ ID NO: 8 - DNA, 25 bp, Synthetic construct
  source 1..25: mol_type = other DNA
tggactcactgctgggaccatctac

SEQ ID NO: 9 - DNA, 21 bp, Synthetic construct
  source 1..21: mol_type = other DNA
ttactagccatcggcttcatt

SEQ ID NO: 10 - DNA, 20 bp, Synthetic construct
  source 1..20: mol_type = other DNA
ggcgagtaccttcccgacat

SEQ ID NO: 11 - DNA, 20 bp, Synthetic construct
  source 1..20: mol_type = other DNA
aacagcgacacccactcctc

SEQ ID NO: 12 - DNA, 24 bp, Synthetic construct
  source 1..24: mol_type = other DNA
cataccaggaaatgagcttgacaa